SEQUENCE LISTING (189 sequences)

SEQ ID NO: 1 - DNA, 1652 bp, Artificial Sequence
  Polynucleotide encoding a polysaccharide degrading enzyme
gtgaatcccttcgtcaagacggcgcgcgtggctatcacctcgacgctggtggcaggctcgctcgccactgccagcctcgtgtttgcaccacttgcacaggccgattactcccccctgcaggagggccccctgaacaagcgcgcctacccctccttcgaggcctactcgaactacaaggtggaccgcaccgacctggaaaccttcctggacaagcagaaggaggtgtcgctgtattacctgctgcagaacatcgcctaccccgagggccagttcaataatggcgtgcccggcaccgtgatcgcctcgccctcgacctcgaatcccgattactactaccagtggacccgtgactcagccatcaccttcctgaccgtgctgtcggagctggaggacaacaacttcaacaccaccctggccaaggccgtggagtactacatcaacacctcgtacaacctgcagcgcacctcgaacccctccggctcgttcgacgacgagaatcacaagggcctgggcgagccaaagtttaataccgacggttcggcctataccggcgcatggggccgtccccaaaatgatggtcccgcactgcgtgcctacgccatctcgcgctacctgaacgacgtgaattcgctgaacgagggcaagctggtgctgaccgactcgggcgacatcaacttctcgtccaccgaggacatctacaagaacatcatcaagcccgacctggagtacgtgatcggctactgggactcgaccggcttcgacctgtgggaggagaatcagggccgtcacttcttcacctcgctggttcagcagaaggccctggcctatgccgtggatatcgccaagtcgttcgatgacggcgacttcgccaataccctgtcgtcgactgcctcgaccctggagtcgtacctgtcgggttcggacggcggcttcgtgaacactgacgtgaaccacatcgtggagaaccccgatctgctgcaacagaattcgcgccagggcctggactcggccacctacatcggtcccctgttgacccatgatatcggcgagtcatcgtcgacccccttcgacgtggacaacgagtacgtgctgcagtcgtactacctgctgctggaggacaataaggaccgctactccgtgaactcggcctactcggccggcgccgccatcggtcgttatccagaggatgtgtataatggcgacggctcctcagaaggcaatccctggttcctggccaccgcatacgccgcacaggttccctacaagctggcctacgacgccaagtcagcctcgaatgacatcaccatcaacaagatcaactacgacttcttcaacaagtacatcgtggacctgtcgaccatcaactccgcctaccagtcgtcggactcggtgaccatcaagtcgggctcggacgagttcaacaccgtggccgacaacctggtgaccttcggcgactcgttcctgcaggtcatcctggaccacatcaacgacgacggctcgctgaatgagcagctgaaccgttacaccggctactccaccggtgcctactcgctgacctggtcatcgggcgccttgttggaggccattcgtctgcgcaataaggtgaaggccctggcctgatgatgccgatcacctacaactgaatcccatgg

SEQ ID NO: 2 - DNA, 2006 bp, Artificial Sequence
  Polynucleotide encoding a polysaccharide degrading enzyme
gtgaatcccttcgtcaagacggcgcgcgtggctatcacctcgacgctggtggcaggctcgctcgccactgccagcctcgtgtttgcaccacttgcacaggccgattactcccccatctcgaagcgtgccacctgggactcgtggttgtcgaatgaggccaccgtggcccgcactgccatcctgaataatattggtgccgacggtgcctgggtgtcgggtgccgattcaggtattgtggtggcctcgccctcgactgacaatcccgactacttctacacctggacccgcgactcgggcttggtgctgaagaccctggtggacctgttccgcaacggcgacacctcgctgctgtcgaccatcgagaattacatctcggcccaggccatcgtgcagggtatctcgaatccctcgggcgacctgtcatcgggcgccggcttgggtgagccaaagttcaatgtggatgaaactgcctacactggctcgtggggccgtccccaaagagacggccccgccttacgtgccaccgccatgatcggtttcggtcagtggctgttggacaatggctacacctccactgccaccgacatcgtgtggcccctggtgcgcaatgacctgtcgtacgtggcccagtactggaaccagaccggctacgacctgtgggaggtgaatggctcgtcgttcttcaccattgcagtgcagcaccgtgccctggtggaaggttcggccttcgcaaccgccgtcggctcctcgtgctcatggtgcgattcgcaagcacccgaaatcctgtgctacctgcagtcgttctggaccggctcgttcattctggccaacttcgactcgtcgcgctccgccaaggacgccaatactctgctgctgggctcaatccacaccttcgaccccgaggccgcatgcgacgactcaaccttccaaccctgttcaccccgcgcactggccaatcacaaggaggtggtggactcgttccgctcgatctacaccttgaatgacggcctgtcagactcggaagccgtggccgtgggccgctaccccgaggacacttactataatggcaatccctggttcctgtgcaccttggccgcagccgagcagctgtacgacgcactgtatcagtgggacaagcagggctcgctggaggtgaccgatgtgtcgctggacttcttcaaggccctgtattcggacgccaccggcacctactcctcctcgtcgtcgacctactcgtccatcgtggacgccgtgaagaccttcgcagacggcttcgtgtcgatcgtggagactcacgccgcctcgaatggctccatgtcggagcagtacgacaagtcggatggcgaacagctgtcggcccgcgacctgacctggtcatatgccgccttgctgactgccaataaccgtcgtaatgtggtgccctcagcctcctggggtgagacgtcggcctcgtcggtgcccggtacttgtgcagccacctcagccattggcacctactcatcagtgaccgtcacttcatggccatcgattgtggcaaccggcggcaccactaccaccgccaccccaactggctccggttcggttacttccacctcgaagaccaccgcaaccgcctcaaagacctcaacctcgacctcatccacctcatgcaccacccccaccgccgtggcagtgaccttcgacctgaccgccaccactacctacggcgagaatatctacctggtgggttcgatctcgcagctgggcgactgggagacgtcagatggcatcgccctgtcagcagacaagtacacctcgtcagatcccctgtggtatgtgaccgtgaccctgcccgccggcgagtcgttcgagtacaagttcatccgcatcgaatcggacgattcagtggagtgggagtccgatcccaatcgtgagtacaccgtgccacaggcctgcggcacctcaaccgccaccgtgaccgacacctggcgctagtgatgccgatcacctacaactgaatcccatgg

SEQ ID NO: 3 - DNA, 2165 bp, Artificial Sequence
  Polynucleotide encoding a polysaccharide degrading enzyme
gtgaatcccttcgtcaagacggcgcgcgtggctatcacctcgacgctggtggcaggctcgctcgccactgccagcctcgtgtttgcaccacttgcacaggccgattactcccccatcgaccgcttcaacaacatctccgccgtgaacggccccggcgaggaggatacctgggcctccgcccagaagcaaggtgtgggcaccgccaataattacgtgtcaaaggtgtggttcaccctggcaaacggcgccatctcggaggtgtactaccccaccatcgacaccgccgacgtgaaggagatcaagttcatcgtgaccgacggcaagtcgttcgtgtcggacgagacgaaggacaccatctcgaaggtggagaagttcaccgacaagtcgctgggctacaagctggtgaacaccgacaagaagggccgctaccgcatcaccaaggagatcttcaccgacgtgaagcgcaactccctgatcatgaaggccaagttcgaggccctggagggctcgatccacgactacaagctgtacctggcctacgacccccacatcaagaaccagggctcgtacaatgagggctacgtgatcaaggccaacaacaacgagatgctgatggccaagcgcgacaacgtgtacaccgccctgtcgtcgaatatcggctggaagggctactcgatcggctactacaaggtgaacgacatcatgaccgacctggacgagaacaagcagatgaccaagcactacgactccgcccgcggcaacatcatcgagggcgccgagatcgacctgaagaagaactcgcagttcgagatcgtgctgtcgttcggcaactccgaggacgaggccgtgaaggcctccatcgagacgctgtcggagaattacgactcgctgaagtcggcctacatcgacgagtgggagaagtactgcaactcgctgaacaacttcaacggcaaggccaattcgctgtactacaactccatgatgatcctgaaggcctcggaggacaagaccaataagggcgcctacatcgcctcgctgtcgatcccctggggcgacggtcagggcgacgataataccggcggttaccaccttgtgtggtcacgtgatctgtaccacgtggccaatgccttcatcgccgccggtgacgtggactcggccaaccgctcgctggactacctggccaaggtggtgaaggacaatggcatgattccccagaatacctggatctccggcaagccctactggaccggcatccagctggacgagcaagccgaccccattatcctgtcgtaccgcctgcgccgctatgacctgtatgactcgttggtgaagcccctggccgacttcatcatcaagatgggccccaaaactggtcaggaacgttgggaggagattggtggctattcacccgcaaccatggccgccgaggtggccggcttgacctgtgccgcctacatcgccgagcagaataaggactacgagtcggcccagaagtatcaggagaaggccgacaactggcagaagctgatcgacaacctgacctacaccgagcacggccccctggagaacggtcagtactacatccgcattgccggcttgcccgatcccaatgccgacttcaccatctccatcgccaatggtggcggcgtgtatgaccagaaggagatcgtggacccctcgttcctggagctggttcgcctgggcgtgaagtcaccagacgaccccaagattctgaacaccctgcgcgtggtggactccaccatcaaggtggatacccccaagggcccctcgtggtaccgctacaaccatgacggctacggcgaaccatcgaagaccgaactgtaccatggcgcaggtaagggtcgtttgtggcccctgttgaccggcgagcgcggcatgtacgaaatcgccgcaggcaaggatgccactccctatctgaaggccatggagaatttcgccaacgagggcggcattatctcggaacaagtgtgggaggataccggtctgcccaccgactcagcctcgcccttgaattgggcccacgccgagtacgtggtgctgttcccctcgaacatcgaacacaaggtgctggacatgcccgacatcgtgtacaagcgctacgtggccaagtgatgatgccgatcacctacaactgaatcccatgg

SEQ ID NO: 4 - DNA, 1238 bp, Artificial Sequence
  Polynucleotide encoding a polysaccharide degrading enzyme
gtgaatcccttcgtcaagacggcgcgcgtggctatcacctcgacgctggtggcaggctcgctcgccactgccagcctcgtgtttgcaccacttgcacaggccgattactcccccatggagaacctggtcgagaagtcgatcaagatcatcaagaacaaccagtcggagtacggctccttcatcgcctcaccctcgttccccacctaccacttctcgtggttgcgcgacggctcgttcatcgcctactccatggacctggtggagcaatacgccgaggccaagaagttctaccgctgggtgaatgaggtgatcatccgctactcgtacaaggtggacaagatcatcgagaagatcaagaacggcaacaagctggagcccaatgacttcctgtacgcccgctataccctggagggctacgaggagaaggattcgggctggggcaatttccaactggatggctacggcacctggctgtggggcctgtcggaacacatcaagatcaccggcaagaccgagctgatcaacgacttcttcaagtccatcgacatcaccatcaagtacatcgacaacctgtggtactaccccaacttcgacgtgtgggaggagaactccgacaagatccacacctcgaccctggcctgcctgtacggcggcctgaactcgatcaacaagtacctgaacgacgacaaggtgaaggagctggccaacaagatcaagacctacatcctgaccaactgcgtggtggagaactcgttcgtgaagtacgtgggctcgaactcggtggactcgtcgctgatctggctggccatccccttcgaggtggtggacgtgaatgacgagatcttcctgaacaccatcaagcgcatcgagaaggagctgctgcacaatggcggcatgcaccgttaccgcaaggacacctactacggcggcggccagtggattctgctgtccgcctggatgggtctgtactactgcaagtcgggcgactacaagaaggccgaggaggtgaagaagtggatcgaggagcaggccgacgagaacggctacctgcccgagcaggtcccctaccacctgaacaacgaggtgtactacccctactgggtgaacaagtggggcaacatcgccaagcccctgctgtggtcgcacgccatgtacctggtgctggactacgagctgaagaaggccggcgtgcagctggaggactgatgatgccgatcacctacaactgaatcccatgg

SEQ ID NO: 5 - DNA, 2183 bp, Artificial Sequence
  Polynucleotide encoding a polysaccharide degrading enzyme
gtgaatcccttcgtcaagacggcgcgcgtggctatcacctcgacgctggtggcaggctcgctcgccactgccagcctcgtgtttgcaccacttgcacaggccgattactcccccaacatctccaacatcaagatcgagcgcctgaataacgtgcaggccgtgaatggccccggtgaggccgacacttgggccaaagcccagaagcagggtgtgggcaccgccaacaattacacctccaaggtgtggttcaccatcgcagacggtggcatctccgaggtgtactaccccaccatcgacaccgccgacgtcaaggacatcaagttcttcgtgaccgacggcaagaccttcgtgtccgacgagacgaaggacaccatcaccaaggtggagaagttcaccgagaagtccctgggctacaagatcatcaacaccgacaaggagggccgctacaagatcaccaaggagatcttcaccgacgtgaagcgcaactccctggtgatcaagaccaagttcgaggccctgaagggcaacgtggacgactaccgcctgtacgtgatgtgcgacccccacgtgaagaaccagggcaagtacaacgagggctacgccgtgaaggccaacggcaatgtggccctgatcgcagagcgcgacggtatctacaccgccctgtcgtccgacatcggttggaagaagtactcgatcggctactacaaggtgaacgacatcgaaaccgacctgtacaagaacatgcagatgacctacaactacgactccgcccgcggcaatatcatcgagggcgccgagatcgacctgaagaagaaccgccagttcgagatcgtgttgtcgttcggccagtcggaggacgaggccgtgaagaccaatatggagacgctgaatgacaattacgactcgctgaagaaggcctacatcgaccagtgggagaagtactgcgactccctgaacgacttcggcggcaaggccaactcgctgtacttcaactccatgatgatcctgaaggcctccgaggacaagaccaacaagggcgcctacatcgcatcgctgtcgatcccctggggtgacggccaggaagacgacaatatcggcggctaccacctggtttggtcccgcgacttgtaccacgtggccaatgccttcattgtggccggcgataccgactcggccaatcgcgccttggactacctggacaaggtggtgaaggacaacggcatgatcccccagaacacctggatcaatggccgcccctactggaccggcattcagctggacgaacaggccgaccccatcatcctgtcataccgcctgaagcgctacgacctgtacgagtcgctggtgaagcccctggccgacttcatcatgaagatcggccccaagaccggccaggaaagatgggaggaaattggtggctactcacccgccaccctggcatcggaagttgccggcctgacctgtgcagcctacatcgccgagcagaacaaggacttcgagtccgccaagaagtaccaggagaaggccgacaactggcagcgcctgatcgacaatctgacctacaccgaaaagggccccttgggcgacggccactactacatccgcattgccggcctgcccgatcccaatgccgacttcatgatctcgatcgccaatggtggcggtgtgtacgaccaaaaggagatcgtggacccctcgttcctggagctggtgcgcctgggtgttaagtccgccgacgaccccaagatcctgaacaccctgaaggtggtggacgagactatcaaggtggacacccccaagggcccctcgtggtatcgttataatcacgacggctacggcgagatgtcgaagaccgaactgtaccacggcaccggcaagggccgtctgtggcccctgttgaccggtgaaagaggtatgtatgagatcgccgccggtaaggacgccacctcgtatgtgaaggccatggagaacttcgccaatgagtccggcatcatctcggaacaggtgtgggaagataccggcctgcccactgactccgcctcacccctgaattgggcccacgccgagtacgtgatcctgttcgcctccaatatcgagcacaaggtgctggacatgcccgacatcgtgtacaagcgctacgcctcgaagtgatgatgccgatcacctacaactgaatcccatgg

SEQ ID NO: 6 - DNA, 2183 bp, Artificial Sequence
  Polynucleotide encoding a polysaccharide degrading enzyme
gtgaatcccttcgtcaagacggcgcgcgtggctatcacctcgacgctggtggcaggctcgctcgccactgccagcctcgtgtttgcaccacttgcacaggccgattactccccctcatacgtgaaggtggagcatctggataagaccgaggcctcccagggtcccggcgaacgtgatacctgggccaccgcccaaaagcagggtatcggcaccgccaataatgacgtttcgaaggtttggttcaccctggcccagggcgccctgtcggagatctactaccccaccatcgaccgcgccaactcgaagttcctgaagttcatcgtgaccgacggcaagaccttcgtggccgacgaaaccaccgacaccgtgtcgaaggtcgagaagatcaacaaccgctcgctggcctaccgcctggtgaatatcgacaagcgcggccgctacaagatcaccaaggagatcttcaccgacccccgccgcaactccgtggtgatgaaggtgcgcttcgaggccctgaagggcaagatggaggactacaagctgtacctggtgtacgacccccacatctcgaaccagggcgccgacaatgagggctacgtggtgaaggccaatggcgagtacggcttcatggcctgccgcaacaacgtgtactcggccctgatgaccgatgccaagtggggctcgtactcggtgggctacaatggcgtgaacgaccccgtgtcggatctgaagaagaacaagaagatgacctacaagttcgaccgcgccaagggcaatatcatcgagggcatcgagatcgacctgcgcgacaagaccgagttcaagaccgtgctgtcgttcggcgagtcggaggaggaggccctgaagaccgccctgtcaactctgaaggactcgtacgaccgcatgctgggcatctacatcgccgagtggaacaagtactgcgacggcctgaagaacttcggcggcgaggccgacgagctgtactacacctcgctgatgttcctgaaggcctccgaggacaagaccaacaaaggcgccttcatcgcctcgctgtcgatcccctggggcgaaggtcagggcgacgaaaataagggcggctaccacctggtgtgggcccgtgatctgtatcacatcgccaatgccttcatcgccgccaaggacatcgactcggccaatcgcgccctggacttcctggccatggtggtggagaagaatggcttcatgccgcagaacacctggatcaacggcgacccctactggaacggcatccagatggacgagcaggccgaccccatcatcctggcctaccacctgaagcgctacgacctgtacgagaagctggtgaagcccctggccgacttcatcgtgagagtgggtcccaagactggccaagaacgttgggaagaagccggtggttattcgccagccaccatggcagcagaggttgccggcctggtttgcgccgccgacatcgccaagcagaataaggatatggagcgcgccaagaagtacctggagacggccgacaagtggcaggagctgatcgacaagctgacctacaccaccaagggcccctacggtaatggccagtactatatccgcatcgccggcctgcccgatcccgacgccgatttcctgatctccatcgcaaatggcggcggcgtgtatgaccaaaaggagatcgtggacccctcgttcctggagctggtgcgcctgggcgtgaaggcctacgacgatcccaagatcctgaataccatctcggtggtggactcgctgctgaaggtgaacacccccaagggcccctcgtggtaccgctacaaccacgacggttacggtgaaccagccaagggcgaactgtatcatggtaagggcaagggtcgcctgtggccactgttaaccggcgagcgcggtatgtacgagatcgccgccggcaagaaggccgacgactacctggagtacatgcgcaatttcgccaacgagggcttcgtgctgtcagagcaaatctgggaggacaccggcttgcccaccgattcggcctcccccctgaattgggcacacgccgagtatgtggtgctgttcgcctccaatatcgagggcaaggtggtggacatgccccagatcgtgtacaagcgctacgtgctgggcgagcgctgatgatgccgatcacctacaactgaatcccatgg

SEQ ID NO: 7 - DNA, 2057 bp, Artificial Sequence
  Polynucleotide encoding a polysaccharide degrading enzyme
gtgaatcccttcgtcaagacggcgcgcgtggctatcacctcgacgctggtggcaggctcgctcgccactgccagcctcgtgtttgcaccacttgcacaggccgattactcccccatggtgcgctacatccccatgggcaacggcaagatcctggtgtccttcaacaacgactacaacctgaccgacttctacttctcgaaggacatggccgagaaccactccgccggcaagcccttccgctacggcgtgtcgatcaacgacaagttcacctggatcaacgcctcgaacatcgtgtcgaaggactactacgaccacaccatgatcggcatcgtgaagtacaacatcaacgacgtgtcgttcgaggacgacaacttcgtggacatctacgaggacgtgtacgcccgcaagatcaagatcaccaacaagcgcaaggagaagatcaacgtgaagctgttcttccaccagaacttctccatctacggcaacaacatcggcgacaccggcttctactaccccgacaccaacgccatcgtgcactacaagggccgccgctacttcatcatctcgaccaccgacggcaagaactcgttcgaccagtacgccatcggcatcaaggacttcaacggcatggccggcacctggaaggacgccgaggacaataatttgtcgatgggccccatcgccatcggctcggtggactccaccattcgtcactcgatcgacatcgatcccgagtcgcagaaggagctgtactactacatcgcctgcgcccgcgacttggacaccgtgctgcgcatcaaccgcaacatgtcgaccggcaatctggaccgcatgatgaagcgcaccgagaatttctgggagctgtgggtgtcgaagtcgcccctgaatctgaccgccgagctgaacgagatgtacaagaagtcgctgttcatcatccgctcgcacatcaatgagaagggcgccgtgatggcctcctcggattcggatatcctgcgctcgaatatggactcgtactactactcatggccccgcgacgccggctacgcagccatctcaatgatcgtgtccgagcactcggaccccgccaagctgttcttcgacttctgcatcaacaccatctccaaggacggctacttctaccacaagtacaaccccgacggcaagattgcctcgtcgtggctgccctatatcatggacggcaagcgcatcctgcccatccaggaggacgagtcagccatcgtgatcatcgccgcctggtactactactcgaccaacaatgacatcgagtacatctcctacctgtacgagcgcctgatcaagcgcgtggccgagttcctgtacaacttcacctacgacaacggtctgcccaaggagtcattcgacctgtgggaggagcgcttcggcatccacacctacaccgtggcctcggtgtacatcgccctgatctatgccgccaatttcgccgagatcttcaacgagacggacctggcccgcaagtacaagtccaaggccgacaagatgctggagtcgttcgagaagatgttctactcggacgacctgggctactacgcccgccgcatctacaacggcgacgtggacttcgtgctggactcatcggtgctgtggctggtgatcttcggcatcaagaagcccgatgacccacgcatcgtgtccaccgtgaaggccatcgagaagaagctgtgggtgcccggcatcggtggtatcgccagatacgagggcgactactaccagcgcatctcgggcaagaatatccccggcaacccctggatcatcactaccctgtggctggccgactattacatcatggccggcaataccggccgcgccctggagctgatcaactgggtggttcagcactcagaggagtcgggcatcctgtcggagcagatcaatcccgataatggcgagcccatctcggtgtcccccctgatctggtcgcactcgcagctggtgctgactctgaagcgctacaaggacgccatcaagaacaagggcatccagtgatgatgccgatcacctacaactgaatcccatgg

SEQ ID NO: 8 - DNA, 2567 bp, Artificial Sequence
  Polynucleotide encoding a polysaccharide degrading enzyme
gtgaatcccttcgtcaagacggcgcgcgtggctatcacctcgacgctggtggcaggctcgctcgccactgccagcctcgtgtttgcaccacttgcacaggccgattactcccccaattcgtacaagtcgtccgtggtgcccgtgaagaagaatatctacttcaccggcaacctgtcccgcgacttcgacggcaatatcatcggcatcaacaatcacgcctcgccctggggcgccaacaacgtgatcaagaagctgtacctggcctacaacgaaaccgacctgttcatcggcgtgtacgagaacatctcctacaactcgctgatgatcttcatcaccaacgtgaccaacgacatctacggcacctccaacatctcgaatatgaacacctggaaccgcgacatcaccttcgagggcaccatgaactacttctcggccgtgtacttcgacggcaacaacatcaatcccgccggctacggcacctacgccatcatgtcgggcaagaattcgaccccactggccaagcccatcaactccaccttcgccttcttccccggcaataacaccaccgagatcgccatccccttctcacagatgttccagcacggcttcaatggcaatctgtcgttcggtatttcggccttcgtggtgggcggctcgggcccatgggtgggctcaggcatcccctatcaccagaaaggcatctacaatgacggcaatcaggcctccttcctgatcaacgacaccatcgagatcaacatccccggcatccacgtgtcgcccctgccctcgtacgtggactacccctcctacctgctgatgtccaactggaagaacaatgacttctggatcgccaccggcatccccgactcgtacctgggcaactactcctacgtgcacctgtcgatgcagaccgtgaacatcgagaacaagatcctgcccctgaagttctacttcgacatcttcggccactccatctcggcccgcctgacccccaacaacaccatcaacgtgcgcaccaacaactcggagttcgagctgatcgaccccccctacatgaatcagaccgacgtgatccagaagtccaagtccaccatctactcgtacatctccatgccctacatcaagtacaacgcctccggcaaccagatctcgctgtacaagtacaatctgaccctgttcttcgtgaactcctcgtacaatatcaacaacgactcgggcatgctgaatatctcgatcgtgtcgggccccggcacctcgatcatcggcctgtcgaccaactcgacctacatgaacctggactacatcaaggacgtgaaccacgccaacatcatgaagtggctgtccaagtcgaaggtgtcgatctccggccgctacttgatcgagtacaacatgtcgcagctgctggtgaaggacgaccagaaccccttcaccggcgaaatcgtggcctccccctcccccgtttacttctataattgggtgcgcgacgcctcgttctccgccatctcactgcaggactcaggccacattcgctcagccatgaagtattgggatttcatggccggcgtgcagggcgtggacacctataatggcacctggcagacccgcttcaatttctggaatggctccgtggccggcttcgtgtatcccgagttcgactcggtgggcctgttcgagatcggcatctacaacctgttccaggtgacccacaatatctccgtgatccagaagttcatgcccaacatcctggcctcgctggagttccaggagaagtccatcaagaagtacggcttcatcgccgaggaccactcgatctgggagatggagtacggctactggttctggacccaggccatcaattatctgggcattcgcgacatctcaagaatgcccatgatccagaatcacaatgccgttcccgtgccgcccggccccgtgccccccggtaacatgaataccgccatgatcgcccacaagctgaagtcgaacatcatcaagtacttctacatggacaacatcttcgcccagtacctggtgcccgtcaccaaccagtacggcgacaacaactacacctacttcatggccaacaataccccggactcgtcgcagatcctgcccattgccatgggcttcatcaacccctcgtcccccatggccacctccatcgtgcacaacatctacatgatcctgtggaactaccgcgtgggtggcctgccccgctactacaatgacctgtaccactacaccgaatacggcggctaccacgaatcatcaggtccctcgcccccctggatcatcaccactctgttcctggccctgtacgacgagaagaccggtaatatgaccggcgccctgaatctgatgaagtggtcgtactcgcactcgcagtcgtggctgctgcccgaggccgtggaccccaatttcggctcggtgattgcatcaacctcaccactgacctggtcatcagccatgtatattatcgtttcgctgaattaccgcgccatgccccccgccccgctgcccccaggccccccctgatgatgccgatcacctacaactgaatcccatgg

SEQ ID NO: 9 - DNA, 2316 bp, Artificial Sequence
  Polynucleotide encoding a polysaccharide degrading enzyme
atgcgcacgttgaaaaccttcgccctggcctcggccgcgaccctgatcgccaccgccgcccaggccgcgcctacgacctgggcctattcggccaagaccggtgtcggcgcctcctacgaggcctatgtcgacggcgcctacaaggccggcggcaagaccggggcggtgtcgaaggtctggttctcgatcgccgacggggtcctcaccgagacgatgtacggcctgatccatgaggcgcagatcaagcagatgcgcgtcgccgtcgagacggcgagcggcctggccattgagggggcggatacgacctccaagaccgagtatctgcatgtggacgccgccggccggccgctgtctccggcctacaagatcaccacgaccgatcgccaaggccggtttgtcatcgaaaagcgcatcttcaccgatcccgaccgcaatgcgctgttcgtgcgcgtgacggtgacggccctgaaaggcgcggtcacgccgaccctgctgctggagccgcacatggccaacaccggcggtggcgatgtgggcgcggcctcggcctcggcgctcaccgcccacgagggcaaggcgttcctcagcctgaaggccagcaagccctttgcgaaagcctcggccagcgttctgaaggatggcgatgctctggcggctttgaaggcgaccacgacttcggccaagggcgcgatcgtgctggccggagagctgcccaaggtcgccaagtccgagacctttgacgtggtgatcggcttcggcgctgacgccaaggccgccgaccggaccgccgccgcgacgctgaagacgggctacgccgaggtcctggcccgctacaacggcgagggcgcgcatgtgggctgggaggactatctggcctcgctgaacgaactgccgcgactgcgcgacgcgtccgaggacggcggcaagcttctccaggccagcgctctgatgctcaaggtgcaggaagaccgcacctatgccggcgccctgattgcctcgctgtccaacccctggggcgacacggtcgacgcgaccaagtcctcgaccggctacaaggccgtctggccgcgcgacttctatcagtgcgccatggccttggcggccctgggcgacaaggagacgccgctggcggccttccactatctgcccacggtccaggtcgggcccaagacgccgggcaacaagggcgacggcggctggttcctgcagaagtcgcacgtcgacggcacgcccgagtgggtcggggtccagctcgaccagacggccatgccgatcatgctgggctggaagctctggacgtggggctggctgccggacgccgagctgaaggcgttctacggcaagatgctgaagccggcggccgacttcctggtcaagggcggcaaggtcaatctggactggaacaccgcgaccatcactccgcccttcacccagcaggagcgctgggaagaacagggcggccactcgccctcgacgaccgccgccgtcattgcaggcctggtggtcgccggcgacatcgccgaggccgccggcgacgccggctcggccgaactctatcgcaagaccgccgacgactgcgccggcaagctcgaggcccggatggtcacgacgaaggggaccttcggcgacggccactattatctgcgtctcaacagtgaccaggacccgaacaacaagagcccggtcgaggcgcgtaacggccaggctccggtggccgaggacaagatgctggacgcgggcttcctggagctggtgcgctatggcgttcgccgcgccgatgatccggcgatcctggccagcctgccggagattgatgacgaggccctcgaagacctctaccgcgttcgctacagcttcacgttccctggcgtggagggcagcttccccggctggcgccgctacggcgtcgacggctacggcgaggacaccaagaccggcgccaactacggcgccgacaaccagatgcgtcccggccaaaggggccgggtgtggccgatcttcaccggcgagcgtgggcactacgagctggccgtggcgggcctctcgggcaagcccgatcccaccgccgtgcagaagatccgcgatacctacgtcaaggcgatggagttgttcgccaacgagggactgatgatccccgagcaggtctgggacggcgtcgggaccgacagcgctcatggctatgtccggggcgaggggaccgattcggccacgcccctggcctggagccacgcggagtacgtcaagctgctgcgatcggtcagcgacgggcaggtctgggatcactatgcgcccgtaaaggcgcggtacgcgcgctag

SEQ ID NO: 10 - DNA, 1539 bp, Artificial Sequence
  Polynucleotide encoding a polysaccharide degrading enzyme
atgaaacaacaaaaacggctttacgcccgattgctgacgctgttatttgcgctcatcttcttgctgcctcattctgcagcagcggcggcaaatcttaatgggacgctgatgcagtattttgaatggtacatgcccaatgacggccaacattggaagcgtttgcaaaacgactcggcatatttggctgaacacggtattactgccgtctggattcccccggcatataagggaacgagccaagcggatgtgggctacggtgcttacgacctttatgatttaggggagtttcatcaaaaagggacggttcggacaaagtacggcacaaaaggagagctgcaatctgcgatcaaaagtcttcattcccgcgacattaacgtttacggggatgtggtcatcaaccacaaaggcggcgctgatgcgaccgaagatgtaaccgcggttgaagtcgatcccgctgaccgcaaccgcgtaatttcaggagaacacctaattaaagcctggacacattttcattttccggggcgcggcagcacatacagcgattttaaatggcattggtaccattttgacggaaccgattgggacgagtcccgaaagctgaaccgcatctataagtttcaaggaaaggcttgggattgggaagtttccaatgaaaacggcaactatgattatttgatgtatgccgacatcgattatgaccatcctgatgtcgcagcagaaattaagagatggggcacttggtatgccaatgaactgcaattggacggtttccgtcttgatgctgtcaaacacattaaattttcttttttgcgggattgggttaatcatgtcagggaaaaaacggggaaggaaatgtttacggtagctgaatattggcagaatgacttgggcgcgctggaaaactatttgaacaaaacaaattttaatcattcagtgtttgacgtgccgcttcattatcagttccatgctgcatcgacacagggaggcggctatgatatgaggaaattgctgaacggtacggtcgtttccaagcatccgttgaaatcggttacatttgtcgataaccatgatacacagccggggcaatcgcttgagtcgactgtccaaacatggtttaagccgcttgcttacgcttttattctcacaagggaatctggataccctcaggttttctacggggatatgtacgggacgaaaggagactcccagcgcgaaattcctgccttgaaacacaaaattgaaccgatcttaaaagcgagaaaacagtatgcgtacggagcacagcatgattatttcgaccaccatgacattgtcggctggacaagggaaggcgacagctcggttgcaaattcaggtttggcggcattaataacagacggacccggtggggcaaagcgaatgtatgtcggccggcaaaacgccggtgagacatggcatgacattaccggaaaccgttcggagccggttgtcatcaattcggaaggctggggagagtttcacgtaaacggcgggtcggtttcaatttatgttcaaagatag

SEQ ID NO: 11 - DNA, 1359 bp, Artificial Sequence
  Polynucleotide encoding a polysaccharide degrading enzyme
gagtcgaccctgcgcgagctggccgcccagaacggcggccgccacttcggcaccgccatcgcctactcgcccctgaactcggacgcccagtaccgcaacatcgccgccacccagttctcggccatcacccacgagaacgagatgaagtgggagtcgctggagccccagcgcggccagtacaactggtcgcaggccgacaacatcatcaacttcgccaaggccaacaaccagatcgtgcgcggccacaccctggtgtggcactcgcagctgccctcgtggctgaacaacggcggcttctcgggctcgcagctgcgctcgatcatggagaaccacatcgaggtggtggccggccgctaccgcggcgacgtgtacgcctgggacgtggtgaacgaggccttcaacgaggacggcaccctgcgcgactcgatctggtaccgcggcatgggccgcgactacatcgcccacgccttccgcaaggcccacgaggtggaccccgacgccaagctgtacatcaacgactacaacatcgagggcatcaacgccaagtcgaacggcctgtacaacctggtggtggacctgctgcgcgacggcgtgcccatccacggcatcggcatccagtcgcacctgatcgtgggccaggtgccctcgaccttccagcagaacatccagcgcttcgccgacctgggcctggacgtggccatcaccgagctggacatccgcatgcagatgcccgccgaccagtacaagctgcagcagcaggcccgcgactacgaggccgtggtgaacgcctgcctggccgtgacccgctgcatcggcatcaccgtgtggggcatcgacgacgagcgctcgtgggtgccctacaccttccccggcgagggcgcccccctgctgtacgacggccagtacaaccgcaagcccgcctggtacgccgtgtacgaggccctgggcggcgactcgtcgggcggcggccccggcgagcccggcggccccggcggccccggcgagcccggcggccccggcggccccggcgagcccggcggccccggcgacggcacctgcgccgtgaactacaccgtggtgaacgactggggccacggcatgcagggcgccatcaccgtgtcgaacaccggctcgtcgcccatcaacaactggaccctgcagttctcgttctcgggcgtgaacatctcgaacggctggaacggcgagtggtcgcagtcgggctcgcagatcaccgtgcgcgcccccgcctggaactcgaccctgcagcccggccagtcggtggagctgggcttcgtggccgacaagaccggcaacgtgtcgcccccctcgcagttcaccctgaacggcgccacctgctcg

SEQ ID NO: 12 - DNA, 1230 bp, Artificial Sequence
  Polynucleotide encoding a polysaccharide degrading enzyme
aacgactcgcccttctacgtgaaccccaacatgtcgtcggccgagtgggtgcgcaacaaccccaacgacccccgcacccccgtgatccgcgaccgcatcgcctcggtgccccagggcacctggttcgcccaccacaaccccggccagatcaccggccaggtggacgccctgatgtcggccgcccaggccgccggcaagatccccatcctggtggtgtacaacgcccccggccgcgactgcggcaaccactcgtcgggcggcgccccctcgcactcggcctaccgctcgtggatcgacgagttcgccgccggcctgaagaaccgccccgcctacatcatcgtggagcccgacctgatctcgctgatgtcgtcgtgcatgcagcacgtgcagcaggaggtgctggagaccatggcctacgccggcaaggccctgaaggccggctcgtcgcaggcccgcatctacttcgacgccggccactcggcctggcactcgcccgcccagatggcctcgtggctgcagcaggccgacatctcgaactcggcccacggcatcgccaccaacacctcgaactaccgctggaccgccgacgaggtggcctacgccaaggccgtgctgtcggccatcggcaacccctcgctgcgcgccgtgatcgacacctcgcgcaacggcaacggccccgccggcaacgagtggtgcgacccctcgggccgcgccatcggcaccccctcgaccaccaacaccggcgaccccatgatcgacgccttcctgtggatcaagctgcccggcgaggccgacggctgcatcgccggcgccggccagttcgtgccccaggccgcctacgagatggccatcgccgccggcggcaccaaccccaaccccaaccccaaccccacccccacccccacccccacccccacccccccccccggctcgtcgggcgcctgcaccgccacctacaccatcgccaacgagtggaacgacggcttccaggccaccgtgaccgtgaccgccaaccagaacatcaccggctggaccgtgacctggaccttcaccgacggccagaccatcaccaacgcctggaacgccgacgtgtcgacctcgggctcgtcggtgaccgcccgcaacgtgggccacaacggcaccctgtcgcagggcgcctcgaccgagttcggcttcgtgggctcgaagggcaactcgaactcggtgcccaccctgacctgcgccgcctcg

SEQ ID NO: 13 - DNA, 2070 bp, Artificial Sequence
  Polynucleotide encoding a polysaccharide degrading enzyme
gccggcacctacaactacggcgaggccctgcagaagtcgatcatgttctacgagttccagcgctcgggcgacctgcccgccgacaagcgcgacaactggcgcgacgactcgggcatgaaggacggctcggacgtgggcgtggacctgaccggcggctggtacgacgccggcgaccacgtgaagttcaacctgcccatgtcgtacacctcggccatgctggcctggtcgctgtacgaggacaaggacgcctacgacaagtcgggccagaccaagtacatcatggacggcatcaagtgggccaacgactacttcatcaagtgcaaccccacccccggcgtgtactactaccaggtgggcgacggcggcaaggaccactcgtggtggggccccgccgaggtgatgcagatggagcgcccctcgttcaaggtggacgcctcgaagcccggctcggccgtgtgcgcctcgaccgccgcctcgctggcctcggccgccgtggtgttcaagtcgtcggaccccacctacgccgagaagtgcatctcgcacgccaagaacctgttcgacatggccgacaaggccaagtcggacgccggctacaccgccgcctcgggctactactcgtcgtcgtcgttctacgacgacctgtcgtgggccgccgtgtggctgtacctggccaccaacgactcgacctacctggacaaggccgagtcgtacgtgcccaactggggcaaggagcagcagaccgacatcatcgcctacaagtggggccagtgctgggacgacgtgcactacggcgccgagctgctgctggccaagctgaccaacaagcagctgtacaaggactcgatcgagatgaacctggacttctggaccaccggcgtgaacggcacccgcgtgtcgtacacccccaagggcctggcctggctgttccagtggggctcgctgcgccacgccaccacccaggccttcctggccggcgtgtacgccgagtgggagggctgcaccccctcgaaggtgtcggtgtacaaggacttcctgaagtcgcagatcgactacgccctgggctcgaccggccgctcgttcgtggtgggctacggcgtgaaccccccccagcacccccaccaccgcaccgcccacggctcgtggaccgaccagatgacctcgcccacctaccaccgccacaccatctacggcgccctggtgggcggccccgacaacgccgacggctacaccgacgagatcaacaactacgtgaacaacgagatcgcctgcgactacaacgccggcttcaccggcgccctggccaagatgtacaagcactcgggcggcgaccccatccccaacttcaaggccatcgagaagatcaccaacgacgaggtgatcatcaaggccggcctgaactcgaccggccccaactacaccgagatcaaggccgtggtgtacaaccagaccggctggcccgcccgcgtgaccgacaagatctcgttcaagtacttcatggacctgtcggagatcgtggccgccggcatcgaccccctgtcgctggtgacctcgtcgaactactcggagggcaagaacaccaaggtgtcgggcgtgctgccctgggacgtgtcgaacaacgtgtactacgtgaacgtggacctgaccggcgagaacatctaccccggcggccagtcggcctgccgccgcgaggtgcagttccgcatcgccgccccccagggcaccacctactggaaccccaagaacgacttctcgtacgacggcctgcccaccacctcgaccgtgaacaccgtgaccaacatccccgtgtacgacaacggcgtgaaggtgttcggcaacgagcccgccggcggctcggagaaccccgaccccgagatcctgtacggcgacgtgaactcggacaagaacgtggacgccctggacttcgccgccctgaagaagtacctgctgggcggcacctcgtcgatcgacgtgaaggccgccgacacctacaaggacggcaacatcgacgccatcgacatggccaccctgaagaagtacctgctgggcaccatcacccagctgccccagggc

SEQ ID NO: 14 - protein, 519 aa, Artificial Sequence
  Polypeptide, an amylase
MKFGVLFSVFAAIVSALPLQEGPLNKRAYPSFEAYSNYKVDRTDLETFLDKQKEVSLYYLLQNIAYPEGQFNNGVPGTVIASPSTSNPDYYYQWTRDSAITFLTVLSELEDNNFNTTLAKAVEYYINTSYNLQRTSNPSGSFDDENHKGLGEPKFNTDGSAYTGAWGRPQNDGPALRAYAISRYLNDVNSLNEGKLVLTDSGDINFSSTEDIYKNIIKPDLEYVIGYWDSTGFDLWEENQGRHFFTSLVQQKALAYAVDIAKSFDDGDFANTLSSTASTLESYLSGSDGGFVNTDVNHIVENPDLLQQNSRQGLDSATYIGPLLTHDIGESSSTPFDVDNEYVLQSYYLLLEDNKDRYSVNSAYSAGAAIGRYPEDVYNGDGSSEGNPWFLATAYAAQVPYKLAYDAKSASNDITINKINYDFFNKYIVDLSTINSAYQSSDSVTIKSGSDEFNTVADNLVTFGDSFLQVILDHINDDGSLNEQLNRYTGYSTGAYSLTWSSGALLEAIRLRNKVKALA

SEQ ID NO: 15 - protein, 639 aa, Artificial Sequence
  Polypeptide, an amylase
MSFRSLLALSGLVCTGLANVISKRATWDSWLSNEATVARTAILNNIGADGAWVSGADSGIVVASPSTDNPDYFYTWTRDSGLVLKTLVDLFRNGDTSLLSTIENYISAQAIVQGISNPSGDLSSGAGLGEPKFNVDETAYTGSWGRPQRDGPALRATAMIGFGQWLLDNGYTSTATDIVWPLVRNDLSYVAQYWNQTGYDLWEVNGSSFFTIAVQHRALVEGSAFATAVGSSCSWCDSQAPEILCYLQSFWTGSFILANFDSSRSAKDANTLLLGSIHTFDPEAACDDSTFQPCSPRALANHKEVVDSFRSIYTLNDGLSDSEAVAVGRYPEDTYYNGNPWFLCTLAAAEQLYDALYQWDKQGSLEVTDVSLDFFKALYSDATGTYSSSSSTYSSIVDAVKTFADGFVSIVETHAASNGSMSEQYDKSDGEQLSARDLTWSYAALLTANNRRNVVPSASWGETSASSVPGTCAATSAIGTYSSVTVTSWPSIVATGGTTTTATPTGSGSVTSTSKTTATASKTSTSTSSTSCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR

SEQ ID NO: 16 - protein, 702 aa, Artificial Sequence
  Polypeptide, an amylase
MSRKLIKYLPLLVLASSVLSGCSNNVSSIKIDRFNNISAVNGPGEEDTWASAQKQGVGTANNYVSKVWFTLANGAISEVYYPTIDTADVKEIKFIVTDGKSFVSDETKDTISKVEKFTDKSLGYKLVNTDKKGRYRITKEIFTDVKRNSLIMKAKFEALEGSIHDYKLYLAYDPHIKNQGSYNEGYVIKANNNEMLMAKRDNVYTALSSNIGWKGYSIGYYKVNDIMTDLDENKQMTKHYDSARGNIIEGAEIDLKKNSQFEIVLSFGNSEDEAVKASIETLSENYDSLKSAYIDEWEKYCNSLNNFNGKANSLYYNSMMILKASEDKTNKGAYIASLSIPWGDGQGDDNTGGYHLVWSRDLYHVANAFIAAGDVDSANRSLDYLAKVVKDNGMIPQNTWISGKPYWTGIQLDEQADPIILSYRLRRYDLYDSLVKPLADFIIKMGPKTGQERWEEIGGYSPATMAAEVAGLTCAAYIAEQNKDYESAQKYQEKADNWQKLIDNLTYTEHGPLENGQYYIRIAGLPDPNADFTISIANGGGVYDQKEIVDPSFLELVRLGVKSPDDPKILNTLRVVDSTIKVDTPKGPSWYRYNHDGYGEPSKTELYHGAGKGRLWPLLTGERGMYEIAAGKDATPYLKAMENFANEGGIISEQVWEDTGLPTDSASPLNWAHAEYVVLFPSNIEHKVLDMPDIVYKRYVAK

SEQ ID NO: 17 - protein, 363 aa, Artificial Sequence
  Polypeptide, an amylase
MENLVEKSIKIIKNNQSEYGSFIASPSFPTYHFSWLRDGSFIAYSMDLVEQYAEAKKFYRWVNEVIIRYSYKVDKIIEKIKNGNKLEPNDFLYARYTLEGYEEKDSGWGNFQLDGYGTWLWGLSEHIKITGKTELINDFFKSIDITIKYIDNLWYYPNFDVWEENSDKIHTSTLACLYGGLNSINKYLNDDKVKELANKIKTYILTNCVVENSFVKYVGSNSVDSSLIWLAIPFEVVDVNDEIFLNTIKRIEKELLHNGGMHRYRKDTYYGGGQWILLSAWMGLYYCKSGDYKKAEEVKKWIEEQADENGYLPEQVPYHLNNEVYYPYWVNKWGNIAKPLLWSHAMYLVLDYELKKAGVQLED

SEQ ID NO: 18 - protein, 710 aa, Artificial Sequence
  Polypeptide, an amylase
MLLEAMKLNRKLIKYLPVLFLASSVLSGCANNNISNIKIERLNNVQAVNGPGEADTWAKAQKQGVGTANNYTSKVWFTIADGGISEVYYPTIDTADVKDIKFFVTDGKTFVSDETKDTITKVEKFTEKSLGYKIINTDKEGRYKITKEIFTDVKRNSLVIKTKFEALKGNVDDYRLYVMCDPHVKNQGKYNEGYAVKANGNVALIAERDGIYTALSSDIGWKKYSIGYYKVNDIETDLYKNMQMTYNYDSARGNIIEGAEIDLKKNRQFEIVLSFGQSEDEAVKTNMETLNDNYDSLKKAYIDQWEKYCDSLNDFGGKANSLYFNSMMILKASEDKTNKGAYIASLSIPWGDGQEDDNIGGYHLVWSRDLYHVANAFIVAGDTDSANRALDYLDKVVKDNGMIPQNTWINGRPYWTGIQLDEQADPIILSYRLKRYDLYESLVKPLADFIMKIGPKTGQERWEEIGGYSPATLASEVAGLTCAAYIAEQNKDFESAKKYQEKADNWQRLIDNLTYTEKGPLGDGHYYIRIAGLPDPNADFMISIANGGGVYDQKEIVDPSFLELVRLGVKSADDPKILNTLKVVDETIKVDTPKGPSWYRYNHDGYGEMSKTELYHGTGKGRLWPLLTGERGMYEIAAGKDATSYVKAMENFANESGIISEQVWEDTGLPTDSASPLNWAHAEYVILFASNIEHKVLDMPDIVYKRYASK

SEQ ID NO: 19 - protein, 703 aa, Artificial Sequence
  Polypeptide, an amylase
MLKRIGILLIFIVIGVFVLSGCSDVSYVKVEHLDKTEASQGPGERDTWATAQKQGIGTANNDVSKVWFTLAQGALSEIYYPTIDRANSKFLKFIVTDGKTFVADETTDTVSKVEKINNRSLAYRLVNIDKRGRYKITKEIFTDPRRNSVVMKVRFEALKGKMEDYKLYLVYDPHISNQGADNEGYVVKANGEYGFMACRNNVYSALMTDAKWGSYSVGYNGVNDPVSDLKKNKKMTYKFDRAKGNIIEGIEIDLRDKTEFKTVLSFGESEEEALKTALSTLKDSYDRMLGIYIAEWNKYCDGLKNFGGEADELYYTSLMFLKASEDKTNKGAFIASLSIPWGEGQGDENKGGYHLVWARDLYHIANAFIAAKDIDSANRALDFLAMVVEKNGFMPQNTWINGDPYWNGIQMDEQADPIILAYHLKRYDLYEKLVKPLADFIVRVGPKTGQERWEEAGGYSPATMAAEVAGLVCAADIAKQNKDMERAKKYLETADKWQELIDKLTYTTKGPYGNGQYYIRIAGLPDPDADFLISIANGGGVYDQKEIVDPSFLELVRLGVKAYDDPKILNTISVVDSLLKVNTPKGPSWYRYNHDGYGEPAKGELYHGKGKGRLWPLLTGERGMYEIAAGKKADDYLEYMRNFANEGFVLSEQIWEDTGLPTDSASPLNWAHAEYVVLFASNIEGKVVDMPQIVYKRYVLGER

SEQ ID NO: 20 - protein, 636 aa, Artificial Sequence
  Polypeptide, an amylase
MVRYIPMGNGKILVSFNNDYNLTDFYFSKDMAENHSAGKPFRYGVSINDKFTWINASNIVSKDYYDHTMIGIVKYNINDVSFEDDNFVDIYEDVYARKIKITNKRKEKINVKLFFHQNFSIYGNNIGDTGFYYPDTNAIVHYKGRRYFIISTTDGKNSFDQYAIGIKDFNGMAGTWKDAEDNNLSMGPIAIGSVDSTIRHSIDIDPESQKELYYYIACARDLDTVLRINRNMSTGNLDRMMKRTENFWELWVSKSPLNLTAELNEMYKKSLFIIRSHINEKGAVMASSDSDILRSNMDSYYYSWPRDAGYAAISMIVSEHSDPAKLFFDFCINTISKDGYFYHKYNPDGKIASSWLPYIMDGKRILPIQEDESAIVIIAAWYYYSTNNDIEYISYLYERLIKRVAEFLYNFTYDNGLPKESFDLWEERFGIHTYTVASVYIALIYAANFAEIFNETDLARKYKSKADKMLESFEKMFYSDDLGYYARRIYNGDVDFVLDSSVLWLVIFGIKKPDDPRIVSTVKAIEKKLWVPGIGGIARYEGDYYQRISGKNIPGNPWIITTLWLADYYIMAGNTGRALELINWVVQHSEESGILSEQINPDNGEPISVSPLIWSHSQLVLTLKRYKDAIKNKGIQ

SEQ ID NO: 21 - protein, 808 aa, Artificial Sequence
  Polypeptide, an amylase
HENSYKSSVVPVKKNIYFTGNLSRDFDGNIIGINNHASPWGANNVIKKLYLAYNETDLFIGVYENISYNSLMIFITNVTNDIYGTSNISNMNTWNRDITFEGTMNYFSAVYFDGNNINPAGYGTYAIMSGKNSTPLAKPINSTFAFFPGNNTTEIAIPFSQMFQHGFNGNLSFGISAFVVGGSGPWVGSGIPYHQKGIYNDGNQASFLINDTIEINIPGIHVSPLPSYVDYPSYLLMSNWKNNDFWIATGIPDSYLGNYSYVHLSMQTVNIENKILPLKFYFDIFGHSISARLTPNNTINVRTNNSEFELIDPPYMNQTDVIQKSKSTIYSYISMPYIKYNASGNQISLYKYNLTLFFVNSSYNINNDSGMLNISIVSGPGTSIIGLSTNSTYMNLDYIKDVNHANIMKWLSKSKVSISGRYLIEYNMSQLLVKDDQNPFTGEIVASPSPVYFYNWVRDASFSAISLQDSGHIRSAMKYWDFMAGVQGVDTYNGTWQTRFNFWNGSVAGFVYPEFDSVGLFEIGIYNLFQVTHNISVIQKFMPNILASLEFQEKSIKKYGFIAEDHSIWEMEYGYWFWTQAINYLGIRDISRMPMIQNHNAVPVPPGPVPPGNMNTAMIAHKLKSNIIKYFYMDNIFAQYLVPVTNQYGDNNYTYFMANNTPDSSQILPIAMGFINPSSPMATSIVHNIYMILWNYRVGGLPRYYNDLYHYTEYGGYHESSGPSPPWIITTLFLALYDEKTGNMTGALNLMKWSYSHSQSWLLPEAVDPNFGSVIASTSPLTWSSAMYIIVSLNYRAMPPAPLPPGPP

SEQ ID NO: 22 - protein, 771 aa, Artificial Sequence
  Polypeptide, an amylase
MRTLKTFALASAATLIATAAQAAPTTWAYSAKTGVGASYEAYVDGAYKAGGKTGAVSKVWFSIADGVLTETMYGLIHEAQIKQMRVAVETASGLAIEGADTTSKTEYLHVDAAGRPLSPAYKITTTDRQGRFVIEKRIFTDPDRNALFVRVTVTALKGAVTPTLLLEPHMANTGGGDVGAASASALTAHEGKAFLSLKASKPFAKASASVLKDGDALAALKATTTSAKGAIVLAGELPKVAKSETFDVVIGFGADAKAADRTAAATLKTGYAEVLARYNGEGAHVGWEDYLASLNELPRLRDASEDGGKLLQASALMLKVQEDRTYAGALIASLSNPWGDTVDATKSSTGYKAVWPRDFYQCAMALAALGDKETPLAAFHYLPTVQVGPKTPGNKGDGGWFLQKSHVDGTPEWVGVQLDQTAMPIMLGWKLWTWGWLPDAELKAFYGKMLKPAADFLVKGGKVNLDWNTATITPPFTQQERWEEQGGHSPSTTAAVIAGLVVAGDIAEAAGDAGSAELYRKTADDCAGKLEARMVTTKGTFGDGHYYLRLNSDQDPNNKSPVEARNGQAPVAEDKMLDAGFLELVRYGVRRADDPAILASLPEIDDEALEDLYRVRYSFTFPGVEGSFPGWRRYGVDGYGEDTKTGANYGADNQMRPGQRGRVWPIFTGERGHYELAVAGLSGKPDPTAVQKIRDTYVKAMELFANEGLMIPEQVWDGVGTDSAHGYVRGEGTDSATPLAWSHAEYVKLLRSVSDGQVWDHYAPVKARYAR

SEQ ID NO: 23 - protein, 512 aa, Artificial Sequence
  Polypeptide, an amylase
MKQQKRLYARLLTLLFALIFLLPHSAAAAANLNGTLMQYFEWYMPNDGQHWKRLQNDSAYLAEHGITAVWIPPAYKGTSQADVGYGAYDLYDLGEFHQKGTVRTKYGTKGELQSAIKSLHSRDINVYGDVVINHKGGADATEDVTAVEVDPADRNRVISGEHLIKAWTHFHFPGRGSTYSDFKWHWYHFDGTDWDESRKLNRIYKFQGKAWDWEVSNENGNYDYLMYADIDYDHPDVAAEIKRWGTWYANELQLDGFRLDAVKHIKFSFLRDWVNHVREKTGKEMFTVAEYWQNDLGALENYLNKTNFNHSVFDVPLHYQFHAASTQGGGYDMRKLLNGTVVSKHPLKSVTFVDNHDTQPGQSLESTVQTWFKPLAYAFILTRESGYPQVFYGDMYGTKGDSQREIPALKHKIEPILKARKQYAYGAQHDYFDHHDIVGWTREGDSSVANSGLAALITDGPGGAKRMYVGRQNAGETWHDITGNRSEPVVINSEGWGEFHVNGGSVSIYVQR

SEQ ID NO: 24 - protein, 491 aa, Artificial Sequence
  Polypeptide, a xylanase
MTETRHRPSRRARRSLSLLLTSALTAAGLLVTAAPAQAESTLRELAAQNGGRHFGTAIAYSPLNSDAQYRNIAATQFSAITHENEMKWESLEPQRGQYNWSQADNIINFAKANNQIVRGHTLVWHSQLPSWLNNGGFSGSQLRSIMENHIEVVAGRYRGDVYAWDVVNEAFNEDGTLRDSIWYRGMGRDYIAHAFRKAHEVDPDAKLYINDYNIEGINAKSNGLYNLVVDLLRDGVPIHGIGIQSHLIVGQVPSTFQQNIQRFADLGLDVAITELDIRMQMPADQYKLQQQARDYEAVVNACLAVTRCIGITVWGIDDERSWVPYTFPGEGAPLLYDGQYNRKPAWYAVYEALGGDSSGGGPGEPGGPGGPGEPGGPGGPGEPGGPGDGTCAVNYTVVNDWGHGMQGAITVSNTGSSPINNWTLQFSFSGVNISNGWNGEWSQSGSQITVRAPAWNSTLQPGQSVELGFVADKTGNVSPPSQFTLNGATCS

SEQ ID NO: 25 - protein, 441 aa, Artificial Sequence
  Polypeptide, a cellulase
MSPRPLRALLGAAAAALVSAAALAFPSQAAANDSPFYVNPNMSSAEWVRNNPNDPRTPVIRDRIASVPQGTWFAHHNPGQITGQVDALMSAAQAAGKIPILVVYNAPGRDCGNHSSGGAPSHSAYRSWIDEFAAGLKNRPAYIIVEPDLISLMSSCMQHVQQEVLETMAYAGKALKAGSSQARIYFDAGHSAWHSPAQMASWLQQADISNSAHGIATNTSNYRWTADEVAYAKAVLSAIGNPSLRAVIDTSRNGNGPAGNEWCDPSGRAIGTPSTTNTGDPMIDAFLWIKLPGEADGCIAGAGQFVPQAAYEMAIAAGGTNPNPNPNPTPTPTPTPTPPPGSSGACTATYTIANEWNDGFQATVTVTANQNITGWTVTWTFTDGQTITNAWNADVSTSGSSVTARNVGHNGTLSQGASTEFGFVGSKGNSNSVPTLTCAAS

SEQ ID NO: 26 - protein, 725 aa, Artificial Sequence
  Polypeptide, a cellulase
MLKTKRKLTKAIGVALSISILSSLVSFIPQTNTYAAGTYNYGEALQKSIMFYEFQRSGDLPADKRDNWRDDSGMKDGSDVGVDLTGGWYDAGDHVKFNLPMSYTSAMLAWSLYEDKDAYDKSGQTKYIMDGIKWANDYFIKCNPTPGVYYYQVGDGGKDHSWWGPAEVMQMERPSFKVDASKPGSAVCASTAASLASAAVVFKSSDPTYAEKCISHAKNLFDMADKAKSDAGYTAASGYYSSSSFYDDLSWAAVWLYLATNDSTYLDKAESYVPNWGKEQQTDIIAYKWGQCWDDVHYGAELLLAKLTNKQLYKDSIEMNLDFWTTGVNGTRVSYTPKGLAWLFQWGSLRHATTQAFLAGVYAEWEGCTPSKVSVYKDFLKSQIDYALGSTGRSFVVGYGVNPPQHPHHRTAHGSWTDQMTSPTYHRHTIYGALVGGPDNADGYTDEINNYVNNEIACDYNAGFTGALAKMYKHSGGDPIPNFKAIEKITNDEVIIKAGLNSTGPNYTEIKAVVYNQTGWPARVTDKISFKYFMDLSEIVAAGIDPLSLVTSSNYSEGKNTKVSGVLPWDVSNNVYYVNVDLTGENIYPGGQSACRREVQFRIAAPQGTTYWNPKNDFSYDGLPTTSTVNTVTNIPVYDNGVKVFGNEPAGGSENPDPEILYGDVNSDKNVDALDFAALKKYLLGGTSSIDVKAADTYKDGNIDAIDMATLKKYLLGTITQLPQG

SEQ ID NO: 27 - DNA, 420 bp, Artificial Sequence
  Promoter
ccaccgtgagctgcacctgaatcagctgagaatgccctgaaccctcaaacccgcaccctgaacctcaaccttgcgttgaacccgaggggtgcgggttggacgcgcaccccgaccactgcaccccgcggggcgcccctgtgacgaccatggtctgattcacgcctgaaatcactccccgccggggtggagaaccacgtcaacgcggccgtggatcacatcgggcgtcgaaaaacaaccccccatttccccaaccctcaacctgatcctgcactgttgtcgggtttgctgagagccgcctaagctgccgcacgttgtcccagttggggcgtggcctgctgcatacggggccgggaaagacgcctcacctgggatgacgcggaccattggacacggcctttccggccgcgggaaggaccagac

SEQ ID NO: 28 - DNA, 413 bp, Artificial Sequence
  Promoter
ccgtgagctgcacctgaatcagctgagaatgccctgaaccctcaaacccgcaccctgaacctcaaccttgcgttgaacccgaggggtgcgggttggacgcgcaccccgaccactgcaccccgcggggcgcccctgtgacgaccatggtctgattcacgcctgaaatcactccccgccggggtggggaagcacgtcaacacggccttggatcacatcgggcgtcgaaaaacaacccccatttccccaaccctcaacctgatcctgcactgttgtcgggtttgctgagagccgcctaagttgccgcacgttgtcccagttggggcgtggcctgctgcatacggggccggggacgcctcacctgggatgacgcggaccattggacacggcctttccggccgcgggaaggaccagac

SEQ ID NO: 29 - DNA, 145 bp, Artificial Sequence
  Promoter
caccggcccggcagcgtgcatgcgtgcatttccaccctcaagaaccattgactggcgacgcgcaggtgggagaattgaactgacaacacagcggttgtcacccggcccgcaaggccgcggccgcatcggttccgaaggaggaatc

SEQ ID NO: 30 - DNA, 362 bp, Artificial Sequence
  Promoter
tcggtcgggtccggacgggcggtccaacccgccccatttcgtgggcaacatgccccatctcctgcccgggggatggggcatgaccatgccgcgtccgggttggtcaccgcgcgccgcatgtgccggcgatgacaacgtgatttgccatccggggggtcttttgcctcagcgtgaaccgcacagctagaattggtcagcgccgtgtacgccgctccagacctagtgtgtggcgtcgttgtcgatggtccggcccgtccctgcggacggacgggaattatcgggagcgaatcgccgttccgttcagcgggatgcgtttgcgcgcgtcacaatctcacatccatccaacatcggagccctactac

SEQ ID NO: 31 - DNA, 549 bp, Artificial Sequence
  Promoter
ccccgaggtccacacgccgggccgaggcgggtacacctgccgggcaggccgggggttccggctctgggaatggagctgttcggttatccaccatgtggacgacacgcaggcaccatgctcgtcttcgagcggaatcacgggccccgataactgtgtgatttgtgcgtgaaggccgattcgggggccgtttgagcggcgcgccgcacggataacagcgttctatgccaccgataacaccaatagtccccaagtgcttgacaggactcttcgaagcctctttgaagatggctctgactaggggaaacagtcctcagtagtcaccgcgaaggagcagtggctctgttactgttctagcggctggacgggtagttcgattgatgatgctcttgcctcggcatagtgcgtcgccaatcctagggaacccatcgaaggccatgaattttcccagaggacatgggtgttcgaacatcccggcagagctcttgaaggtcggccgtgcacccttgtccaggcttgaaaggaaacaagttgtctgttcgtaggattgcg

SEQ ID NO: 32 - DNA, 315 bp, Artificial Sequence
  Promoter
ctcgagttgcagggcgagggggcggggccgcaggtccatgctggcagcggccggcgacaatcccgacgggagagtcggtcagggcgacggtagatcagcgcaggtgctgactagcggtcgtagtcggcctcggccagcccatcgcgggtgcggatcgacgtgtcggcggatgctgggcgggtcggttcttgtgggggtgtgtgcgtgcgccgaacgtctcggaaaatgctgagcgtttcctgagaatcggagacacacctcgtgcattcttagctgtctggcgcagttcatcgtccggacgatatggtttcgggt

SEQ ID NO: 33 - DNA, 2122 bp, Artificial Sequence
  Polynucleotide, expression cassette
ccgaggtccacacgccgggccgaggcgggtacacctgccgggcaggccgggggttccggctctgggaatggagctgttcggttatccaccatgtggacgacacgcaggcaccatgctcgtcttcgagcggaatcacgggccccgataactgtgtgatttgtgcgtgaaggccgattcgggggccgtttgagcggcgcgccgcacggataacagcgttctatgccaccgataacaccaatagtccccaagtgcttgacaggactcttcgaagcctctttgaagatggctctgactaggggaaacagtcctcagtagtcaccgcgaaggagcagtggctctgttactgttctagcggctggacgggtagttcgattgatgatgctcttgcctcggcatagtgcgtcgccaatcctagggaacccatcgaaggccatgaattttcccagaggacatgggtgttcgaacatcccggcagagctcttgaaggtcggccgtgcacccttgtccaggcttgaaaggaaacaagttgtctgttcgtaggattgcggtggccacaggtgctgcggccgccatgtttgtgacgacgtttgcgggcatggcgcctgcgaatgccctgcaggagggccccctgaacaagcgcgcctacccctccttcgaggcctactcgaactacaaggtggaccgcaccgacctggaaaccttcctggacaagcagaaggaggtgtcgctgtattacctgctgcagaacatcgcctaccccgagggccagttcaataatggcgtgcccggcaccgtgatcgcctcgccctcgacctcgaatcccgattactactaccagtggacccgtgactcagccatcaccttcctgaccgtgctgtcggagctggaggacaacaacttcaacaccaccctggccaaggccgtggagtactacatcaacacctcgtacaacctgcagcgcacctcgaacccctccggctcgttcgacgacgagaatcacaagggcctgggcgagccaaagtttaataccgacggttcggcctataccggcgcatggggccgtccccaaaatgatggtcccgcactgcgtgcctacgccatctcgcgctacctgaacgacgtgaattcgctgaacgagggcaagctggtgctgaccgactcgggcgacatcaacttctcgtccaccgaggacatctacaagaacatcatcaagcccgacctggagtacgtgatcggctactgggactcgaccggcttcgacctgtgggaggagaatcagggccgtcacttcttcacctcgctggttcagcagaaggccctggcctatgccgtggatatcgccaagtcgttcgatgacggcgacttcgccaataccctgtcgtcgactgcctcgaccctggagtcgtacctgtcgggttcggacggcggcttcgtgaacactgacgtgaaccacatcgtggagaaccccgatctgctgcaacagaattcgcgccagggcctggactcggccacctacatcggtcccctgttgacccatgatatcggcgagtcatcgtcgacccccttcgacgtggacaacgagtacgtgctgcagtcgtactacctgctgctggaggacaataaggaccgctactccgtgaactcggcctactcggccggcgccgccatcggtcgttatccagaggatgtgtataatggcgacggctcctcagaaggcaatccctggttcctggccaccgcatacgccgcacaggttccctacaagctggcctacgacgccaagtcagcctcgaatgacatcaccatcaacaagatcaactacgacttcttcaacaagtacatcgtggacctgtcgaccatcaactccgcctaccagtcgtcggactcggtgaccatcaagtcgggctcggacgagttcaacaccgtggccgacaacctggtgaccttcggcgactcgttcctgcaggtcatcctggaccacatcaacgacgacggctcgctgaatgagcagctgaaccgttacaccggctactccaccggtgcctactcgctgacctggtcatcgggcgccttgttggaggccattcgtctgcgcaataaggtgaaggccctggcctgatga

SEQ ID NO: 34 - DNA, 2476 bp, Artificial Sequence
  Polynucleotide, expression cassette
ccgaggtccacacgccgggccgaggcgggtacacctgccgggcaggccgggggttccggctctgggaatggagctgttcggttatccaccatgtggacgacacgcaggcaccatgctcgtcttcgagcggaatcacgggccccgataactgtgtgatttgtgcgtgaaggccgattcgggggccgtttgagcggcgcgccgcacggataacagcgttctatgccaccgataacaccaatagtccccaagtgcttgacaggactcttcgaagcctctttgaagatggctctgactaggggaaacagtcctcagtagtcaccgcgaaggagcagtggctctgttactgttctagcggctggacgggtagttcgattgatgatgctcttgcctcggcatagtgcgtcgccaatcctagggaacccatcgaaggccatgaattttcccagaggacatgggtgttcgaacatcccggcagagctcttgaaggtcggccgtgcacccttgtccaggcttgaaaggaaacaagttgtctgttcgtaggattgcggtggccacaggtgctgcggccgccatgtttgtgacgacgtttgcgggcatggcgcctgcgaatgccatctcgaagcgtgccacctgggactcgtggttgtcgaatgaggccaccgtggcccgcactgccatcctgaataatattggtgccgacggtgcctgggtgtcgggtgccgattcaggtattgtggtggcctcgccctcgactgacaatcccgactacttctacacctggacccgcgactcgggcttggtgctgaagaccctggtggacctgttccgcaacggcgacacctcgctgctgtcgaccatcgagaattacatctcggcccaggccatcgtgcagggtatctcgaatccctcgggcgacctgtcatcgggcgccggcttgggtgagccaaagttcaatgtggatgaaactgcctacactggctcgtggggccgtccccaaagagacggccccgccttacgtgccaccgccatgatcggtttcggtcagtggctgttggacaatggctacacctccactgccaccgacatcgtgtggcccctggtgcgcaatgacctgtcgtacgtggcccagtactggaaccagaccggctacgacctgtgggaggtgaatggctcgtcgttcttcaccattgcagtgcagcaccgtgccctggtggaaggttcggccttcgcaaccgccgtcggctcctcgtgctcatggtgcgattcgcaagcacccgaaatcctgtgctacctgcagtcgttctggaccggctcgttcattctggccaacttcgactcgtcgcgctccgccaaggacgccaatactctgctgctgggctcaatccacaccttcgaccccgaggccgcatgcgacgactcaaccttccaaccctgttcaccccgcgcactggccaatcacaaggaggtggtggactcgttccgctcgatctacaccttgaatgacggcctgtcagactcggaagccgtggccgtgggccgctaccccgaggacacttactataatggcaatccctggttcctgtgcaccttggccgcagccgagcagctgtacgacgcactgtatcagtgggacaagcagggctcgctggaggtgaccgatgtgtcgctggacttcttcaaggccctgtattcggacgccaccggcacctactcctcctcgtcgtcgacctactcgtccatcgtggacgccgtgaagaccttcgcagacggcttcgtgtcgatcgtggagactcacgccgcctcgaatggctccatgtcggagcagtacgacaagtcggatggcgaacagctgtcggcccgcgacctgacctggtcatatgccgccttgctgactgccaataaccgtcgtaatgtggtgccctcagcctcctggggtgagacgtcggcctcgtcggtgcccggtacttgtgcagccacctcagccattggcacctactcatcagtgaccgtcacttcatggccatcgattgtggcaaccggcggcaccactaccaccgccaccccaactggctccggttcggttacttccacctcgaagaccaccgcaaccgcctcaaagacctcaacctcgacctcatccacctcatgcaccacccccaccgccgtggcagtgaccttcgacctgaccgccaccactacctacggcgagaatatctacctggtgggttcgatctcgcagctgggcgactgggagacgtcagatggcatcgccctgtcagcagacaagtacacctcgtcagatcccctgtggtatgtgaccgtgaccctgcccgccggcgagtcgttcgagtacaagttcatccgcatcgaatcggacgattcagtggagtgggagtccgatcccaatcgtgagtacaccgtgccacaggcctgcggcacctcaaccgccaccgtgaccgacacctggcgctagtga

SEQ ID NO: 35 - DNA, 2635 bp, Artificial Sequence
  Polynucleotide, expression cassette
ccgaggtccacacgccgggccgaggcgggtacacctgccgggcaggccgggggttccggctctgggaatggagctgttcggttatccaccatgtggacgacacgcaggcaccatgctcgtcttcgagcggaatcacgggccccgataactgtgtgatttgtgcgtgaaggccgattcgggggccgtttgagcggcgcgccgcacggataacagcgttctatgccaccgataacaccaatagtccccaagtgcttgacaggactcttcgaagcctctttgaagatggctctgactaggggaaacagtcctcagtagtcaccgcgaaggagcagtggctctgttactgttctagcggctggacgggtagttcgattgatgatgctcttgcctcggcatagtgcgtcgccaatcctagggaacccatcgaaggccatgaattttcccagaggacatgggtgttcgaacatcccggcagagctcttgaaggtcggccgtgcacccttgtccaggcttgaaaggaaacaagttgtctgttcgtaggattgcggtggccacaggtgctgcggccgccatgtttgtgacgacgtttgcgggcatggcgcctgcgaatgccatcgaccgcttcaacaacatctccgccgtgaacggccccggcgaggaggatacctgggcctccgcccagaagcaaggtgtgggcaccgccaataattacgtgtcaaaggtgtggttcaccctggcaaacggcgccatctcggaggtgtactaccccaccatcgacaccgccgacgtgaaggagatcaagttcatcgtgaccgacggcaagtcgttcgtgtcggacgagacgaaggacaccatctcgaaggtggagaagttcaccgacaagtcgctgggctacaagctggtgaacaccgacaagaagggccgctaccgcatcaccaaggagatcttcaccgacgtgaagcgcaactccctgatcatgaaggccaagttcgaggccctggagggctcgatccacgactacaagctgtacctggcctacgacccccacatcaagaaccagggctcgtacaatgagggctacgtgatcaaggccaacaacaacgagatgctgatggccaagcgcgacaacgtgtacaccgccctgtcgtcgaatatcggctggaagggctactcgatcggctactacaaggtgaacgacatcatgaccgacctggacgagaacaagcagatgaccaagcactacgactccgcccgcggcaacatcatcgagggcgccgagatcgacctgaagaagaactcgcagttcgagatcgtgctgtcgttcggcaactccgaggacgaggccgtgaaggcctccatcgagacgctgtcggagaattacgactcgctgaagtcggcctacatcgacgagtgggagaagtactgcaactcgctgaacaacttcaacggcaaggccaattcgctgtactacaactccatgatgatcctgaaggcctcggaggacaagaccaataagggcgcctacatcgcctcgctgtcgatcccctggggcgacggtcagggcgacgataataccggcggttaccaccttgtgtggtcacgtgatctgtaccacgtggccaatgccttcatcgccgccggtgacgtggactcggccaaccgctcgctggactacctggccaaggtggtgaaggacaatggcatgattccccagaatacctggatctccggcaagccctactggaccggcatccagctggacgagcaagccgaccccattatcctgtcgtaccgcctgcgccgctatgacctgtatgactcgttggtgaagcccctggccgacttcatcatcaagatgggccccaaaactggtcaggaacgttgggaggagattggtggctattcacccgcaaccatggccgccgaggtggccggcttgacctgtgccgcctacatcgccgagcagaataaggactacgagtcggcccagaagtatcaggagaaggccgacaactggcagaagctgatcgacaacctgacctacaccgagcacggccccctggagaacggtcagtactacatccgcattgccggcttgcccgatcccaatgccgacttcaccatctccatcgccaatggtggcggcgtgtatgaccagaaggagatcgtggacccctcgttcctggagctggttcgcctgggcgtgaagtcaccagacgaccccaagattctgaacaccctgcgcgtggtggactccaccatcaaggtggatacccccaagggcccctcgtggtaccgctacaaccatgacggctacggcgaaccatcgaagaccgaactgtaccatggcgcaggtaagggtcgtttgtggcccctgttgaccggcgagcgcggcatgtacgaaatcgccgcaggcaaggatgccactccctatctgaaggccatggagaatttcgccaacgagggcggcattatctcggaacaagtgtgggaggataccggtctgcccaccgactcagcctcgcccttgaattgggcccacgccgagtacgtggtgctgttcccctcgaacatcgaacacaaggtgctggacatgcccgacatcgtgtacaagcgctacgtggccaagtgatga

SEQ ID NO: 36 - DNA, 1708 bp, Artificial Sequence
  Polynucleotide, expression cassette
ccgaggtccacacgccgggccgaggcgggtacacctgccgggcaggccgggggttccggctctgggaatggagctgttcggttatccaccatgtggacgacacgcaggcaccatgctcgtcttcgagcggaatcacgggccccgataactgtgtgatttgtgcgtgaaggccgattcgggggccgtttgagcggcgcgccgcacggataacagcgttctatgccaccgataacaccaatagtccccaagtgcttgacaggactcttcgaagcctctttgaagatggctctgactaggggaaacagtcctcagtagtcaccgcgaaggagcagtggctctgttactgttctagcggctggacgggtagttcgattgatgatgctcttgcctcggcatagtgcgtcgccaatcctagggaacccatcgaaggccatgaattttcccagaggacatgggtgttcgaacatcccggcagagctcttgaaggtcggccgtgcacccttgtccaggcttgaaaggaaacaagttgtctgttcgtaggattgcggtggccacaggtgctgcggccgccatgtttgtgacgacgtttgcgggcatggcgcctgcgaatgccatggagaacctggtcgagaagtcgatcaagatcatcaagaacaaccagtcggagtacggctccttcatcgcctcaccctcgttccccacctaccacttctcgtggttgcgcgacggctcgttcatcgcctactccatggacctggtggagcaatacgccgaggccaagaagttctaccgctgggtgaatgaggtgatcatccgctactcgtacaaggtggacaagatcatcgagaagatcaagaacggcaacaagctggagcccaatgacttcctgtacgcccgctataccctggagggctacgaggagaaggattcgggctggggcaatttccaactggatggctacggcacctggctgtggggcctgtcggaacacatcaagatcaccggcaagaccgagctgatcaacgacttcttcaagtccatcgacatcaccatcaagtacatcgacaacctgtggtactaccccaacttcgacgtgtgggaggagaactccgacaagatccacacctcgaccctggcctgcctgtacggcggcctgaactcgatcaacaagtacctgaacgacgacaaggtgaaggagctggccaacaagatcaagacctacatcctgaccaactgcgtggtggagaactcgttcgtgaagtacgtgggctcgaactcggtggactcgtcgctgatctggctggccatccccttcgaggtggtggacgtgaatgacgagatcttcctgaacaccatcaagcgcatcgagaaggagctgctgcacaatggcggcatgcaccgttaccgcaaggacacctactacggcggcggccagtggattctgctgtccgcctggatgggtctgtactactgcaagtcgggcgactacaagaaggccgaggaggtgaagaagtggatcgaggagcaggccgacgagaacggctacctgcccgagcaggtcccctaccacctgaacaacgaggtgtactacccctactgggtgaacaagtggggcaacatcgccaagcccctgctgtggtcgcacgccatgtacctggtgctggactacgagctgaagaaggccggcgtgcagctggaggactgatga

SEQ ID NO: 37 - DNA, 2653 bp, Artificial Sequence
  Polynucleotide, expression cassette
ccgaggtccacacgccgggccgaggcgggtacacctgccgggcaggccgggggttccggctctgggaatggagctgttcggttatccaccatgtggacgacacgcaggcaccatgctcgtcttcgagcggaatcacgggccccgataactgtgtgatttgtgcgtgaaggccgattcgggggccgtttgagcggcgcgccgcacggataacagcgttctatgccaccgataacaccaatagtccccaagtgcttgacaggactcttcgaagcctctttgaagatggctctgactaggggaaacagtcctcagtagtcaccgcgaaggagcagtggctctgttactgttctagcggctggacgggtagttcgattgatgatgctcttgcctcggcatagtgcgtcgccaatcctagggaacccatcgaaggccatgaattttcccagaggacatgggtgttcgaacatcccggcagagctcttgaaggtcggccgtgcacccttgtccaggcttgaaaggaaacaagttgtctgttcgtaggattgcggtggccacaggtgctgcggccgccatgtttgtgacgacgtttgcgggcatggcgcctgcgaatgccaacatctccaacatcaagatcgagcgcctgaataacgtgcaggccgtgaatggccccggtgaggccgacacttgggccaaagcccagaagcagggtgtgggcaccgccaacaattacacctccaaggtgtggttcaccatcgcagacggtggcatctccgaggtgtactaccccaccatcgacaccgccgacgtcaaggacatcaagttcttcgtgaccgacggcaagaccttcgtgtccgacgagacgaaggacaccatcaccaaggtggagaagttcaccgagaagtccctgggctacaagatcatcaacaccgacaaggagggccgctacaagatcaccaaggagatcttcaccgacgtgaagcgcaactccctggtgatcaagaccaagttcgaggccctgaagggcaacgtggacgactaccgcctgtacgtgatgtgcgacccccacgtgaagaaccagggcaagtacaacgagggctacgccgtgaaggccaacggcaatgtggccctgatcgcagagcgcgacggtatctacaccgccctgtcgtccgacatcggttggaagaagtactcgatcggctactacaaggtgaacgacatcgaaaccgacctgtacaagaacatgcagatgacctacaactacgactccgcccgcggcaatatcatcgagggcgccgagatcgacctgaagaagaaccgccagttcgagatcgtgttgtcgttcggccagtcggaggacgaggccgtgaagaccaatatggagacgctgaatgacaattacgactcgctgaagaaggcctacatcgaccagtgggagaagtactgcgactccctgaacgacttcggcggcaaggccaactcgctgtacttcaactccatgatgatcctgaaggcctccgaggacaagaccaacaagggcgcctacatcgcatcgctgtcgatcccctggggtgacggccaggaagacgacaatatcggcggctaccacctggtttggtcccgcgacttgtaccacgtggccaatgccttcattgtggccggcgataccgactcggccaatcgcgccttggactacctggacaaggtggtgaaggacaacggcatgatcccccagaacacctggatcaatggccgcccctactggaccggcattcagctggacgaacaggccgaccccatcatcctgtcataccgcctgaagcgctacgacctgtacgagtcgctggtgaagcccctggccgacttcatcatgaagatcggccccaagaccggccaggaaagatgggaggaaattggtggctactcacccgccaccctggcatcggaagttgccggcctgacctgtgcagcctacatcgccgagcagaacaaggacttcgagtccgccaagaagtaccaggagaaggccgacaactggcagcgcctgatcgacaatctgacctacaccgaaaagggccccttgggcgacggccactactacatccgcattgccggcctgcccgatcccaatgccgacttcatgatctcgatcgccaatggtggcggtgtgtacgaccaaaaggagatcgtggacccctcgttcctggagctggtgcgcctgggtgttaagtccgccgacgaccccaagatcctgaacaccctgaaggtggtggacgagactatcaaggtggacacccccaagggcccctcgtggtatcgttataatcacgacggctacggcgagatgtcgaagaccgaactgtaccacggcaccggcaagggccgtctgtggcccctgttgaccggtgaaagaggtatgtatgagatcgccgccggtaaggacgccacctcgtatgtgaaggccatggagaacttcgccaatgagtccggcatcatctcggaacaggtgtgggaagataccggcctgcccactgactccgcctcacccctgaattgggcccacgccgagtacgtgatcctgttcgcctccaatatcgagcacaaggtgctggacatgcccgacatcgtgtacaagcgctacgcctcgaagtgatga

SEQ ID NO: 38 - DNA, 2527 bp, Artificial Sequence
  Polynucleotide, expression cassette
ccgaggtccacacgccgggccgaggcgggtacacctgccgggcaggccgggggttccggctctgggaatggagctgttcggttatccaccatgtggacgacacgcaggcaccatgctcgtcttcgagcggaatcacgggccccgataactgtgtgatttgtgcgtgaaggccgattcgggggccgtttgagcggcgcgccgcacggataacagcgttctatgccaccgataacaccaatagtccccaagtgcttgacaggactcttcgaagcctctttgaagatggctctgactaggggaaacagtcctcagtagtcaccgcgaaggagcagtggctctgttactgttctagcggctggacgggtagttcgattgatgatgctcttgcctcggcatagtgcgtcgccaatcctagggaacccatcgaaggccatgaattttcccagaggacatgggtgttcgaacatcccggcagagctcttgaaggtcggccgtgcacccttgtccaggcttgaaaggaaacaagttgtctgttcgtaggattgcggtggccacaggtgctgcggccgccatgtttgtgacgacgtttgcgggcatggcgcctgcgaatgccatggtgcgctacatccccatgggcaacggcaagatcctggtgtccttcaacaacgactacaacctgaccgacttctacttctcgaaggacatggccgagaaccactccgccggcaagcccttccgctacggcgtgtcgatcaacgacaagttcacctggatcaacgcctcgaacatcgtgtcgaaggactactacgaccacaccatgatcggcatcgtgaagtacaacatcaacgacgtgtcgttcgaggacgacaacttcgtggacatctacgaggacgtgtacgcccgcaagatcaagatcaccaacaagcgcaaggagaagatcaacgtgaagctgttcttccaccagaacttctccatctacggcaacaacatcggcgacaccggcttctactaccccgacaccaacgccatcgtgcactacaagggccgccgctacttcatcatctcgaccaccgacggcaagaactcgttcgaccagtacgccatcggcatcaaggacttcaacggcatggccggcacctggaaggacgccgaggacaataatttgtcgatgggccccatcgccatcggctcggtggactccaccattcgtcactcgatcgacatcgatcccgagtcgcagaaggagctgtactactacatcgcctgcgcccgcgacttggacaccgtgctgcgcatcaaccgcaacatgtcgaccggcaatctggaccgcatgatgaagcgcaccgagaatttctgggagctgtgggtgtcgaagtcgcccctgaatctgaccgccgagctgaacgagatgtacaagaagtcgctgttcatcatccgctcgcacatcaatgagaagggcgccgtgatggcctcctcggattcggatatcctgcgctcgaatatggactcgtactactactcatggccccgcgacgccggctacgcagccatctcaatgatcgtgtccgagcactcggaccccgccaagctgttcttcgacttctgcatcaacaccatctccaaggacggctacttctaccacaagtacaaccccgacggcaagattgcctcgtcgtggctgccctatatcatggacggcaagcgcatcctgcccatccaggaggacgagtcagccatcgtgatcatcgccgcctggtactactactcgaccaacaatgacatcgagtacatctcctacctgtacgagcgcctgatcaagcgcgtggccgagttcctgtacaacttcacctacgacaacggtctgcccaaggagtcattcgacctgtgggaggagcgcttcggcatccacacctacaccgtggcctcggtgtacatcgccctgatctatgccgccaatttcgccgagatcttcaacgagacggacctggcccgcaagtacaagtccaaggccgacaagatgctggagtcgttcgagaagatgttctactcggacgacctgggctactacgcccgccgcatctacaacggcgacgtggacttcgtgctggactcatcggtgctgtggctggtgatcttcggcatcaagaagcccgatgacccacgcatcgtgtccaccgtgaaggccatcgagaagaagctgtgggtgcccggcatcggtggtatcgccagatacgagggcgactactaccagcgcatctcgggcaagaatatccccggcaacccctggatcatcactaccctgtggctggccgactattacatcatggccggcaataccggccgcgccctggagctgatcaactgggtggttcagcactcagaggagtcgggcatcctgtcggagcagatcaatcccgataatggcgagcccatctcggtgtcccccctgatctggtcgcactcgcagctggtgctgactctgaagcgctacaaggacgccatcaagaacaagggcatccagtgatga

SEQ ID NO: 39 - DNA, 2043 bp, Artificial Sequence
  Polynucleotide, expression cassette
ccaccgtgagctgcacctgaatcagctgagaatgccctgaaccctcaaacccgcaccctgaacctcaaccttgcgttgaacccgaggggtgcgggttggacgcgcaccccgaccactgcaccccgcggggcgcccctgtgacgaccatggtctgattcacgcctgaaatcactccccgccggggtggagaaccacgtcaacgcggccgtggatcacatcgggcgtcgaaaaacaaccccccatttccccaaccctcaacctgatcctgcactgttgtcgggtttgctgagagccgcctaagctgccgcacgttgtcccagttggggcgtggcctgctgcatacggggccgggaaagacgcctcacctgggatgacgcggaccattggacacggcctttccggccgcgggaaggaccagacgtgaatcccttcgtcaagacggcgcgcgtggctatcacctcgacgctggtggcaggctcgctcgccactgccagcctcgtgtttgcaccacttgcacaggccgattactcccccctgcaggagggccccctgaacaagcgcgcctacccctccttcgaggcctactcgaactacaaggtggaccgcaccgacctggaaaccttcctggacaagcagaaggaggtgtcgctgtattacctgctgcagaacatcgcctaccccgagggccagttcaataatggcgtgcccggcaccgtgatcgcctcgccctcgacctcgaatcccgattactactaccagtggacccgtgactcagccatcaccttcctgaccgtgctgtcggagctggaggacaacaacttcaacaccaccctggccaaggccgtggagtactacatcaacacctcgtacaacctgcagcgcacctcgaacccctccggctcgttcgacgacgagaatcacaagggcctgggcgagccaaagtttaataccgacggttcggcctataccggcgcatggggccgtccccaaaatgatggtcccgcactgcgtgcctacgccatctcgcgctacctgaacgacgtgaattcgctgaacgagggcaagctggtgctgaccgactcgggcgacatcaacttctcgtccaccgaggacatctacaagaacatcatcaagcccgacctggagtacgtgatcggctactgggactcgaccggcttcgacctgtgggaggagaatcagggccgtcacttcttcacctcgctggttcagcagaaggccctggcctatgccgtggatatcgccaagtcgttcgatgacggcgacttcgccaataccctgtcgtcgactgcctcgaccctggagtcgtacctgtcgggttcggacggcggcttcgtgaacactgacgtgaaccacatcgtggagaaccccgatctgctgcaacagaattcgcgccagggcctggactcggccacctacatcggtcccctgttgacccatgatatcggcgagtcatcgtcgacccccttcgacgtggacaacgagtacgtgctgcagtcgtactacctgctgctggaggacaataaggaccgctactccgtgaactcggcctactcggccggcgccgccatcggtcgttatccagaggatgtgtataatggcgacggctcctcagaaggcaatccctggttcctggccaccgcatacgccgcacaggttccctacaagctggcctacgacgccaagtcagcctcgaatgacatcaccatcaacaagatcaactacgacttcttcaacaagtacatcgtggacctgtcgaccatcaactccgcctaccagtcgtcggactcggtgaccatcaagtcgggctcggacgagttcaacaccgtggccgacaacctggtgaccttcggcgactcgttcctgcaggtcatcctggaccacatcaacgacgacggctcgctgaatgagcagctgaaccgttacaccggctactccaccggtgcctactcgctgacctggtcatcgggcgccttgttggaggccattcgtctgcgcaataaggtgaaggccctggcctgatga

SEQ ID NO: 40 - DNA, 2398 bp, Artificial Sequence
  Polynucleotide, expression cassette
ccaccgtgagctgcacctgaatcagctgagaatgccctgaaccctcaaacccgcaccctgaacctcaaccttgcgttgaacccgaggggtgcgggttggacgcgcaccccgaccactgcaccccgcggggcgcccctgtgacgaccatggtctgattcacgcctgaaatcactccccgccggggtggagaaccacgtcaacgcggccgtggatcacatcgggcgtcgaaaaacaaccccccatttccccaaccctcaacctgatcctgcactgttgtcgggtttgctgagagccgcctaagctgccgcacgttgtcccagttggggcgtggcctgctgcatacggggccgggaaagacgcctcacctgggatgacgcggaccattggacacggcctttccggccgcgggaaggaccagacgtgaatcccttcgtcaagacggcgcgcgtggctatcacctcgacgctggtggcaggctcgctcgccactgccagcctcgtgtttgcaccacttgcacaggccgattactcccccatctcgaagcgtgccacctgggactcgtggttgtcgaatgaggccaccgtggcccgcactgccatcctgaataatattggtgccgacggtgcctgggtgtcgggtgccgattcaggtattgtggtggcctcgccctcgactgacaatcccgactacttctacacctggacccgcgactcgggcttggtgctgaagaccctggtggacctgttccgcaacggcgacacctcgctgctgtcgaccatcgagaattacatctcggcccaggccatcgtgcagggtatctcgaatccctcgggcgacctgtcatcgggcgccggcttgggtgagccaaagttcaatgtggatgaaactgcctacactggctcgtggggccgtccccaaagagacggccccgccttacgtgccaccgccatgatcggtttcggtcagtggctgttggacaatggctacacctccactgccaccgacatcgtgtggcccctggtgcgcaatgacctgtcgtacgtggcccagtactggaaccagaccggctacgacctgtgggaggtgaatggctcgtcgttcttcaccattgcagtgcagcaccgtgccctggtggaaggttcggccttcgcaaccgccgtcggctcctcgtgctcatggtgcgattcgcaagcacccgaaatcctgtgctacctgcagtcgttctggaccggctcgttcattctggccaacttcgactcgtcgcgctccgccaaggacgccaatactctgctgctgggctcaatccacaccttcgaccccgaggccgcatgcgacgactcaaccttccaaccctgttcaccccgcgcactggccaatcacaaggaggtggtggactcgttccgctcgatctacaccttgaatgacggcctgtcagactcggaagccgtggccgtgggccgctaccccgaggacacttactataatggcaatccctggttcctgtgcaccttggccgcagccgagcagctgtacgacgcactgtatcagtgggacaagcagggctcgctggaggtgaccgatgtgtcgctggacttcttcaaggccctgtattcggacgccaccggcacctactcctcctcgtcgtcgacctactcgtccatcgtggacgccgtgaagaccttcgcagacggcttcgtgtcgatcgtggagactcacgccgcctcgaatggctccatgtcggagcagtacgacaagtcggatggcgaacagctgtcggcccgcgacctgacctggtcatatgccgccttgctgactgccaataaccgtcgtaatgtggtgccctcagcctcctggggtgagacgtcggcctcgtcggtgcccggtacttgtgcagccacctcagccattggcacctactcatcagtgaccgtcacttcatggccatcgattgtggcaaccggcggcaccactaccaccgccaccccaactggctccggttcggttacttccacctcgaagaccaccgcaaccgcctcaaagacctcaacctcgacctcatccacctcatgcaccacccccaccgccgtggcagtgaccttcgacctgaccgccaccactacctacggcgagaatatctacctggtgggttcgatctcgcagctgggcgactgggagacgtcagatggcatcgccctgtcagcagacaagtacacctcgtcagatcccctgtggtatgtgaccgtgaccctgcccgccggcgagtcgttcgagtacaagttcatccgcatcgaatcggacgattcagtggagtgggagtccgatcccaatcgtgagtacaccgtgccacaggcctgcggcacctcaaccgccaccgtgaccgacacctggcgctagtgat

SEQ ID NO: 41 - DNA, 2556 bp, Artificial Sequence
  Polynucleotide, expression cassette
ccaccgtgagctgcacctgaatcagctgagaatgccctgaaccctcaaacccgcaccctgaacctcaaccttgcgttgaacccgaggggtgcgggttggacgcgcaccccgaccactgcaccccgcggggcgcccctgtgacgaccatggtctgattcacgcctgaaatcactccccgccggggtggagaaccacgtcaacgcggccgtggatcacatcgggcgtcgaaaaacaaccccccatttccccaaccctcaacctgatcctgcactgttgtcgggtttgctgagagccgcctaagctgccgcacgttgtcccagttggggcgtggcctgctgcatacggggccgggaaagacgcctcacctgggatgacgcggaccattggacacggcctttccggccgcgggaaggaccagacgtgaatcccttcgtcaagacggcgcgcgtggctatcacctcgacgctggtggcaggctcgctcgccactgccagcctcgtgtttgcaccacttgcacaggccgattactcccccatcgaccgcttcaacaacatctccgccgtgaacggccccggcgaggaggatacctgggcctccgcccagaagcaaggtgtgggcaccgccaataattacgtgtcaaaggtgtggttcaccctggcaaacggcgccatctcggaggtgtactaccccaccatcgacaccgccgacgtgaaggagatcaagttcatcgtgaccgacggcaagtcgttcgtgtcggacgagacgaaggacaccatctcgaaggtggagaagttcaccgacaagtcgctgggctacaagctggtgaacaccgacaagaagggccgctaccgcatcaccaaggagatcttcaccgacgtgaagcgcaactccctgatcatgaaggccaagttcgaggccctggagggctcgatccacgactacaagctgtacctggcctacgacccccacatcaagaaccagggctcgtacaatgagggctacgtgatcaaggccaacaacaacgagatgctgatggccaagcgcgacaacgtgtacaccgccctgtcgtcgaatatcggctggaagggctactcgatcggctactacaaggtgaacgacatcatgaccgacctggacgagaacaagcagatgaccaagcactacgactccgcccgcggcaacatcatcgagggcgccgagatcgacctgaagaagaactcgcagttcgagatcgtgctgtcgttcggcaactccgaggacgaggccgtgaaggcctccatcgagacgctgtcggagaattacgactcgctgaagtcggcctacatcgacgagtgggagaagtactgcaactcgctgaacaacttcaacggcaaggccaattcgctgtactacaactccatgatgatcctgaaggcctcggaggacaagaccaataagggcgcctacatcgcctcgctgtcgatcccctggggcgacggtcagggcgacgataataccggcggttaccaccttgtgtggtcacgtgatctgtaccacgtggccaatgccttcatcgccgccggtgacgtggactcggccaaccgctcgctggactacctggccaaggtggtgaaggacaatggcatgattccccagaatacctggatctccggcaagccctactggaccggcatccagctggacgagcaagccgaccccattatcctgtcgtaccgcctgcgccgctatgacctgtatgactcgttggtgaagcccctggccgacttcatcatcaagatgggccccaaaactggtcaggaacgttgggaggagattggtggctattcacccgcaaccatggccgccgaggtggccggcttgacctgtgccgcctacatcgccgagcagaataaggactacgagtcggcccagaagtatcaggagaaggccgacaactggcagaagctgatcgacaacctgacctacaccgagcacggccccctggagaacggtcagtactacatccgcattgccggcttgcccgatcccaatgccgacttcaccatctccatcgccaatggtggcggcgtgtatgaccagaaggagatcgtggacccctcgttcctggagctggttcgcctgggcgtgaagtcaccagacgaccccaagattctgaacaccctgcgcgtggtggactccaccatcaaggtggatacccccaagggcccctcgtggtaccgctacaaccatgacggctacggcgaaccatcgaagaccgaactgtaccatggcgcaggtaagggtcgtttgtggcccctgttgaccggcgagcgcggcatgtacgaaatcgccgcaggcaaggatgccactccctatctgaaggccatggagaatttcgccaacgagggcggcattatctcggaacaagtgtgggaggataccggtctgcccaccgactcagcctcgcccttgaattgggcccacgccgagtacgtggtgctgttcccctcgaacatcgaacacaaggtgctggacatgcccgacatcgtgtacaagcgctacgtggccaagtgatga

SEQ ID NO: 42 - DNA, 1629 bp, Artificial Sequence
  Polynucleotide, expression cassette
ccaccgtgagctgcacctgaatcagctgagaatgccctgaaccctcaaacccgcaccctgaacctcaaccttgcgttgaacccgaggggtgcgggttggacgcgcaccccgaccactgcaccccgcggggcgcccctgtgacgaccatggtctgattcacgcctgaaatcactccccgccggggtggagaaccacgtcaacgcggccgtggatcacatcgggcgtcgaaaaacaaccccccatttccccaaccctcaacctgatcctgcactgttgtcgggtttgctgagagccgcctaagctgccgcacgttgtcccagttggggcgtggcctgctgcatacggggccgggaaagacgcctcacctgggatgacgcggaccattggacacggcctttccggccgcgggaaggaccagacgtgaatcccttcgtcaagacggcgcgcgtggctatcacctcgacgctggtggcaggctcgctcgccactgccagcctcgtgtttgcaccacttgcacaggccgattactcccccatggagaacctggtcgagaagtcgatcaagatcatcaagaacaaccagtcggagtacggctccttcatcgcctcaccctcgttccccacctaccacttctcgtggttgcgcgacggctcgttcatcgcctactccatggacctggtggagcaatacgccgaggccaagaagttctaccgctgggtgaatgaggtgatcatccgctactcgtacaaggtggacaagatcatcgagaagatcaagaacggcaacaagctggagcccaatgacttcctgtacgcccgctataccctggagggctacgaggagaaggattcgggctggggcaatttccaactggatggctacggcacctggctgtggggcctgtcggaacacatcaagatcaccggcaagaccgagctgatcaacgacttcttcaagtccatcgacatcaccatcaagtacatcgacaacctgtggtactaccccaacttcgacgtgtgggaggagaactccgacaagatccacacctcgaccctggcctgcctgtacggcggcctgaactcgatcaacaagtacctgaacgacgacaaggtgaaggagctggccaacaagatcaagacctacatcctgaccaactgcgtggtggagaactcgttcgtgaagtacgtgggctcgaactcggtggactcgtcgctgatctggctggccatccccttcgaggtggtggacgtgaatgacgagatcttcctgaacaccatcaagcgcatcgagaaggagctgctgcacaatggcggcatgcaccgttaccgcaaggacacctactacggcggcggccagtggattctgctgtccgcctggatgggtctgtactactgcaagtcgggcgactacaagaaggccgaggaggtgaagaagtggatcgaggagcaggccgacgagaacggctacctgcccgagcaggtcccctaccacctgaacaacgaggtgtactacccctactgggtgaacaagtggggcaacatcgccaagcccctgctgtggtcgcacgccatgtacctggtgctggactacgagctgaagaaggccggcgtgcagctggaggactgatga

SEQ ID NO: 43 - DNA, 2574 bp, Artificial Sequence
  Polynucleotide, expression cassette
ccaccgtgagctgcacctgaatcagctgagaatgccctgaaccctcaaacccgcaccctgaacctcaaccttgcgttgaacccgaggggtgcgggttggacgcgcaccccgaccactgcaccccgcggggcgcccctgtgacgaccatggtctgattcacgcctgaaatcactccccgccggggtggagaaccacgtcaacgcggccgtggatcacatcgggcgtcgaaaaacaaccccccatttccccaaccctcaacctgatcctgcactgttgtcgggtttgctgagagccgcctaagctgccgcacgttgtcccagttggggcgtggcctgctgcatacggggccgggaaagacgcctcacctgggatgacgcggaccattggacacggcctttccggccgcgggaaggaccagacgtgaatcccttcgtcaagacggcgcgcgtggctatcacctcgacgctggtggcaggctcgctcgccactgccagcctcgtgtttgcaccacttgcacaggccgattactcccccaacatctccaacatcaagatcgagcgcctgaataacgtgcaggccgtgaatggccccggtgaggccgacacttgggccaaagcccagaagcagggtgtgggcaccgccaacaattacacctccaaggtgtggttcaccatcgcagacggtggcatctccgaggtgtactaccccaccatcgacaccgccgacgtcaaggacatcaagttcttcgtgaccgacggcaagaccttcgtgtccgacgagacgaaggacaccatcaccaaggtggagaagttcaccgagaagtccctgggctacaagatcatcaacaccgacaaggagggccgctacaagatcaccaaggagatcttcaccgacgtgaagcgcaactccctggtgatcaagaccaagttcgaggccctgaagggcaacgtggacgactaccgcctgtacgtgatgtgcgacccccacgtgaagaaccagggcaagtacaacgagggctacgccgtgaaggccaacggcaatgtggccctgatcgcagagcgcgacggtatctacaccgccctgtcgtccgacatcggttggaagaagtactcgatcggctactacaaggtgaacgacatcgaaaccgacctgtacaagaacatgcagatgacctacaactacgactccgcccgcggcaatatcatcgagggcgccgagatcgacctgaagaagaaccgccagttcgagatcgtgttgtcgttcggccagtcggaggacgaggccgtgaagaccaatatggagacgctgaatgacaattacgactcgctgaagaaggcctacatcgaccagtgggagaagtactgcgactccctgaacgacttcggcggcaaggccaactcgctgtacttcaactccatgatgatcctgaaggcctccgaggacaagaccaacaagggcgcctacatcgcatcgctgtcgatcccctggggtgacggccaggaagacgacaatatcggcggctaccacctggtttggtcccgcgacttgtaccacgtggccaatgccttcattgtggccggcgataccgactcggccaatcgcgccttggactacctggacaaggtggtgaaggacaacggcatgatcccccagaacacctggatcaatggccgcccctactggaccggcattcagctggacgaacaggccgaccccatcatcctgtcataccgcctgaagcgctacgacctgtacgagtcgctggtgaagcccctggccgacttcatcatgaagatcggccccaagaccggccaggaaagatgggaggaaattggtggctactcacccgccaccctggcatcggaagttgccggcctgacctgtgcagcctacatcgccgagcagaacaaggacttcgagtccgccaagaagtaccaggagaaggccgacaactggcagcgcctgatcgacaatctgacctacaccgaaaagggccccttgggcgacggccactactacatccgcattgccggcctgcccgatcccaatgccgacttcatgatctcgatcgccaatggtggcggtgtgtacgaccaaaaggagatcgtggacccctcgttcctggagctggtgcgcctgggtgttaagtccgccgacgaccccaagatcctgaacaccctgaaggtggtggacgagactatcaaggtggacacccccaagggcccctcgtggtatcgttataatcacgacggctacggcgagatgtcgaagaccgaactgtaccacggcaccggcaagggccgtctgtggcccctgttgaccggtgaaagaggtatgtatgagatcgccgccggtaaggacgccacctcgtatgtgaaggccatggagaacttcgccaatgagtccggcatcatctcggaacaggtgtgggaagataccggcctgcccactgactccgcctcacccctgaattgggcccacgccgagtacgtgatcctgttcgcctccaatatcgagcacaaggtgctggacatgcccgacatcgtgtacaagcgctacgcctcgaagtgatga

SEQ ID NO: 44 - DNA, 2448 bp, Artificial Sequence
  Polynucleotide, expression cassette
ccaccgtgagctgcacctgaatcagctgagaatgccctgaaccctcaaacccgcaccctgaacctcaaccttgcgttgaacccgaggggtgcgggttggacgcgcaccccgaccactgcaccccgcggggcgcccctgtgacgaccatggtctgattcacgcctgaaatcactccccgccggggtggagaaccacgtcaacgcggccgtggatcacatcgggcgtcgaaaaacaaccccccatttccccaaccctcaacctgatcctgcactgttgtcgggtttgctgagagccgcctaagctgccgcacgttgtcccagttggggcgtggcctgctgcatacggggccgggaaagacgcctcacctgggatgacgcggaccattggacacggcctttccggccgcgggaaggaccagacgtgaatcccttcgtcaagacggcgcgcgtggctatcacctcgacgctggtggcaggctcgctcgccactgccagcctcgtgtttgcaccacttgcacaggccgattactcccccatggtgcgctacatccccatgggcaacggcaagatcctggtgtccttcaacaacgactacaacctgaccgacttctacttctcgaaggacatggccgagaaccactccgccggcaagcccttccgctacggcgtgtcgatcaacgacaagttcacctggatcaacgcctcgaacatcgtgtcgaaggactactacgaccacaccatgatcggcatcgtgaagtacaacatcaacgacgtgtcgttcgaggacgacaacttcgtggacatctacgaggacgtgtacgcccgcaagatcaagatcaccaacaagcgcaaggagaagatcaacgtgaagctgttcttccaccagaacttctccatctacggcaacaacatcggcgacaccggcttctactaccccgacaccaacgccatcgtgcactacaagggccgccgctacttcatcatctcgaccaccgacggcaagaactcgttcgaccagtacgccatcggcatcaaggacttcaacggcatggccggcacctggaaggacgccgaggacaataatttgtcgatgggccccatcgccatcggctcggtggactccaccattcgtcactcgatcgacatcgatcccgagtcgcagaaggagctgtactactacatcgcctgcgcccgcgacttggacaccgtgctgcgcatcaaccgcaacatgtcgaccggcaatctggaccgcatgatgaagcgcaccgagaatttctgggagctgtgggtgtcgaagtcgcccctgaatctgaccgccgagctgaacgagatgtacaagaagtcgctgttcatcatccgctcgcacatcaatgagaagggcgccgtgatggcctcctcggattcggatatcctgcgctcgaatatggactcgtactactactcatggccccgcgacgccggctacgcagccatctcaatgatcgtgtccgagcactcggaccccgccaagctgttcttcgacttctgcatcaacaccatctccaaggacggctacttctaccacaagtacaaccccgacggcaagattgcctcgtcgtggctgccctatatcatggacggcaagcgcatcctgcccatccaggaggacgagtcagccatcgtgatcatcgccgcctggtactactactcgaccaacaatgacatcgagtacatctcctacctgtacgagcgcctgatcaagcgcgtggccgagttcctgtacaacttcacctacgacaacggtctgcccaaggagtcattcgacctgtgggaggagcgcttcggcatccacacctacaccgtggcctcggtgtacatcgccctgatctatgccgccaatttcgccgagatcttcaacgagacggacctggcccgcaagtacaagtccaaggccgacaagatgctggagtcgttcgagaagatgttctactcggacgacctgggctactacgcccgccgcatctacaacggcgacgtggacttcgtgctggactcatcggtgctgtggctggtgatcttcggcatcaagaagcccgatgacccacgcatcgtgtccaccgtgaaggccatcgagaagaagctgtgggtgcccggcatcggtggtatcgccagatacgagggcgactactaccagcgcatctcgggcaagaatatccccggcaacccctggatcatcactaccctgtggctggccgactattacatcatggccggcaataccggccgcgccctggagctgatcaactgggtggttcagcactcagaggagtcgggcatcctgtcggagcagatcaatcccgataatggcgagcccatctcggtgtcccccctgatctggtcgcactcgcagctggtgctgactctgaagcgctacaaggacgccatcaagaacaagggcatccagtgatga

SEQ ID NO: 45 - DNA, 2340 bp, Artificial Sequence
  Polynucleotide, expression cassette
tcggtcgggtccggacgggcggtccaacccgccccatttcgtgggcaacatgccccatctcctgcccgggggatggggcatgaccatgccgcgtccgggttggtcaccgcgcgccgcatgtgccggcgatgacaacgtgatttgccatccggggggtcttttgcctcagcgtgaaccgcacagctagaattggtcagcgccgtgtacgccgctccagacctagtgtgtggcgtcgttgtcgatggtccggcccgtccctgcggacggacgggaattatcgggagcgaatcgccgttccgttcagcgggatgcgtttgcgcgcgtcacaatctcacatccatccaacatcggagccctactacgtgaatcccttcgtcaagacggcgcgcgtggctatcacctcgacgctggtggcaggctcgctcgccactgccagcctcgtgtttgcaccacttgcacaggccgattactcccccatctcgaagcgtgccacctgggactcgtggttgtcgaatgaggccaccgtggcccgcactgccatcctgaataatattggtgccgacggtgcctgggtgtcgggtgccgattcaggtattgtggtggcctcgccctcgactgacaatcccgactacttctacacctggacccgcgactcgggcttggtgctgaagaccctggtggacctgttccgcaacggcgacacctcgctgctgtcgaccatcgagaattacatctcggcccaggccatcgtgcagggtatctcgaatccctcgggcgacctgtcatcgggcgccggcttgggtgagccaaagttcaatgtggatgaaactgcctacactggctcgtggggccgtccccaaagagacggccccgccttacgtgccaccgccatgatcggtttcggtcagtggctgttggacaatggctacacctccactgccaccgacatcgtgtggcccctggtgcgcaatgacctgtcgtacgtggcccagtactggaaccagaccggctacgacctgtgggaggtgaatggctcgtcgttcttcaccattgcagtgcagcaccgtgccctggtggaaggttcggccttcgcaaccgccgtcggctcctcgtgctcatggtgcgattcgcaagcacccgaaatcctgtgctacctgcagtcgttctggaccggctcgttcattctggccaacttcgactcgtcgcgctccgccaaggacgccaatactctgctgctgggctcaatccacaccttcgaccccgaggccgcatgcgacgactcaaccttccaaccctgttcaccccgcgcactggccaatcacaaggaggtggtggactcgttccgctcgatctacaccttgaatgacggcctgtcagactcggaagccgtggccgtgggccgctaccccgaggacacttactataatggcaatccctggttcctgtgcaccttggccgcagccgagcagctgtacgacgcactgtatcagtgggacaagcagggctcgctggaggtgaccgatgtgtcgctggacttcttcaaggccctgtattcggacgccaccggcacctactcctcctcgtcgtcgacctactcgtccatcgtggacgccgtgaagaccttcgcagacggcttcgtgtcgatcgtggagactcacgccgcctcgaatggctccatgtcggagcagtacgacaagtcggatggcgaacagctgtcggcccgcgacctgacctggtcatatgccgccttgctgactgccaataaccgtcgtaatgtggtgccctcagcctcctggggtgagacgtcggcctcgtcggtgcccggtacttgtgcagccacctcagccattggcacctactcatcagtgaccgtcacttcatggccatcgattgtggcaaccggcggcaccactaccaccgccaccccaactggctccggttcggttacttccacctcgaagaccaccgcaaccgcctcaaagacctcaacctcgacctcatccacctcatgcaccacccccaccgccgtggcagtgaccttcgacctgaccgccaccactacctacggcgagaatatctacctggtgggttcgatctcgcagctgggcgactgggagacgtcagatggcatcgccctgtcagcagacaagtacacctcgtcagatcccctgtggtatgtgaccgtgaccctgcccgccggcgagtcgttcgagtacaagttcatccgcatcgaatcggacgattcagtggagtgggagtccgatcccaatcgtgagtacaccgtgccacaggcctgcggcacctcaaccgccaccgtgaccgacacctggcgctagtgat

SEQ ID NO: 46 - DNA, 2383 bp, Artificial Sequence
  Polynucleotide, expression cassette
ccaccgtgagctgcacctgaatcagctgagaatgccctgaaccctcaaacccgcaccctgaacctcaaccttgcgttgaacccgaggggtgcgggttggacgcgcaccccgaccactgcaccccgcggggcgcccctgtgacgaccatggtctgattcacgcctgaaatcactccccgccggggtggagaaccacgtcaacgcggccgtggatcacatcgggcgtcgaaaaacaaccccccatttccccaaccctcaacctgatcctgcactgttgtcgggtttgctgagagccgcctaagctgccgcacgttgtcccagttggggcgtggcctgctgcatacggggccgggaaagacgcctcacctgggatgacgcggaccattggacacggcctttccggccgcgggaaggaccagacatgctcactcgcaagagagtggttgcagcgggagctgccgccaccctgtccctcacggcgtttgccgggttgcagcccgccagcgccgccaccggccccatctcgaagcgtgccacctgggactcgtggttgtcgaatgaggccaccgtggcccgcactgccatcctgaataatattggtgccgacggtgcctgggtgtcgggtgccgattcaggtattgtggtggcctcgccctcgactgacaatcccgactacttctacacctggacccgcgactcgggcttggtgctgaagaccctggtggacctgttccgcaacggcgacacctcgctgctgtcgaccatcgagaattacatctcggcccaggccatcgtgcagggtatctcgaatccctcgggcgacctgtcatcgggcgccggcttgggtgagccaaagttcaatgtggatgaaactgcctacactggctcgtggggccgtccccaaagagacggccccgccttacgtgccaccgccatgatcggtttcggtcagtggctgttggacaatggctacacctccactgccaccgacatcgtgtggcccctggtgcgcaatgacctgtcgtacgtggcccagtactggaaccagaccggctacgacctgtgggaggtgaatggctcgtcgttcttcaccattgcagtgcagcaccgtgccctggtggaaggttcggccttcgcaaccgccgtcggctcctcgtgctcatggtgcgattcgcaagcacccgaaatcctgtgctacctgcagtcgttctggaccggctcgttcattctggccaacttcgactcgtcgcgctccgccaaggacgccaatactctgctgctgggctcaatccacaccttcgaccccgaggccgcatgcgacgactcaaccttccaaccctgttcaccccgcgcactggccaatcacaaggaggtggtggactcgttccgctcgatctacaccttgaatgacggcctgtcagactcggaagccgtggccgtgggccgctaccccgaggacacttactataatggcaatccctggttcctgtgcaccttggccgcagccgagcagctgtacgacgcactgtatcagtgggacaagcagggctcgctggaggtgaccgatgtgtcgctggacttcttcaaggccctgtattcggacgccaccggcacctactcctcctcgtcgtcgacctactcgtccatcgtggacgccgtgaagaccttcgcagacggcttcgtgtcgatcgtggagactcacgccgcctcgaatggctccatgtcggagcagtacgacaagtcggatggcgaacagctgtcggcccgcgacctgacctggtcatatgccgccttgctgactgccaataaccgtcgtaatgtggtgccctcagcctcctggggtgagacgtcggcctcgtcggtgcccggtacttgtgcagccacctcagccattggcacctactcatcagtgaccgtcacttcatggccatcgattgtggcaaccggcggcaccactaccaccgccaccccaactggctccggttcggttacttccacctcgaagaccaccgcaaccgcctcaaagacctcaacctcgacctcatccacctcatgcaccacccccaccgccgtggcagtgaccttcgacctgaccgccaccactacctacggcgagaatatctacctggtgggttcgatctcgcagctgggcgactgggagacgtcagatggcatcgccctgtcagcagacaagtacacctcgtcagatcccctgtggtatgtgaccgtgaccctgcccgccggcgagtcgttcgagtacaagttcatccgcatcgaatcggacgattcagtggagtgggagtccgatcccaatcgtgagtacaccgtgccacaggcctgcggcacctcaaccgccaccgtgaccgacacctggcgctagtgat

SEQ ID NO: 47 - DNA, 1893 bp, Artificial Sequence
  Polynucleotide, expression cassette
ccaccgtgagctgcacctgaatcagctgagaatgccctgaaccctcaaacccgcaccctgaacctcaaccttgcgttgaacccgaggggtgcgggttggacgcgcaccccgaccactgcaccccgcggggcgcccctgtgacgaccatggtctgattcacgcctgaaatcactccccgccggggtggagaaccacgtcaacgcggccgtggatcacatcgggcgtcgaaaaacaaccccccatttccccaaccctcaacctgatcctgcactgttgtcgggtttgctgagagccgcctaagctgccgcacgttgtcccagttggggcgtggcctgctgcatacggggccgggaaagacgcctcacctgggatgacgcggaccattggacacggcctttccggccgcgggaaggaccagacgtgaatcccttcgtcaagacggcgcgcgtggctatcacctcgacgctggtggcaggctcgctcgccactgccagcctcgtgtttgcaccacttgcacaggccgattactcccccgagtcgaccctgcgcgagctggccgcccagaacggcggccgccacttcggcaccgccatcgcctactcgcccctgaactcggacgcccagtaccgcaacatcgccgccacccagttctcggccatcacccacgagaacgagatgaagtgggagtcgctggagccccagcgcggccagtacaactggtcgcaggccgacaacatcatcaacttcgccaaggccaacaaccagatcgtgcgcggccacaccctggtgtggcactcgcagctgccctcgtggctgaacaacggcggcttctcgggctcgcagctgcgctcgatcatggagaaccacatcgaggtggtggccggccgctaccgcggcgacgtgtacgcctgggacgtggtgaacgaggccttcaacgaggacggcaccctgcgcgactcgatctggtaccgcggcatgggccgcgactacatcgcccacgccttccgcaaggcccacgaggtggaccccgacgccaagctgtacatcaacgactacaacatcgagggcatcaacgccaagtcgaacggcctgtacaacctggtggtggacctgctgcgcgacggcgtgcccatccacggcatcggcatccagtcgcacctgatcgtgggccaggtgccctcgaccttccagcagaacatccagcgcttcgccgacctgggcctggacgtggccatcaccgagctggacatccgcatgcagatgcccgccgaccagtacaagctgcagcagcaggcccgcgactacgaggccgtggtgaacgcctgcctggccgtgacccgctgcatcggcatcaccgtgtggggcatcgacgacgagcgctcgtgggtgccctacaccttccccggcgagggcgcccccctgctgtacgacggccagtacaaccgcaagcccgcctggtacgccgtgtacgaggccctgggcggcgactcgtcgggcggcggccccggcgagcccggcggccccggcggccccggcgagcccggcggccccggcggccccggcgagcccggcggccccggcgacggcacctgcgccgtgaactacaccgtggtgaacgactggggccacggcatgcagggcgccatcaccgtgtcgaacaccggctcgtcgcccatcaacaactggaccctgcagttctcgttctcgggcgtgaacatctcgaacggctggaacggcgagtggtcgcagtcgggctcgcagatcaccgtgcgcgcccccgcctggaactcgaccctgcagcccggccagtcggtggagctgggcttcgtggccgacaagaccggcaacgtgtcgcccccctcgcagttcaccctgaacggcgccacctgctcg

SEQ ID NO: 48 - DNA, 1764 bp, Artificial Sequence
  Polynucleotide, expression cassette
ccaccgtgagctgcacctgaatcagctgagaatgccctgaaccctcaaacccgcaccctgaacctcaaccttgcgttgaacccgaggggtgcgggttggacgcgcaccccgaccactgcaccccgcggggcgcccctgtgacgaccatggtctgattcacgcctgaaatcactccccgccggggtggagaaccacgtcaacgcggccgtggatcacatcgggcgtcgaaaaacaaccccccatttccccaaccctcaacctgatcctgcactgttgtcgggtttgctgagagccgcctaagctgccgcacgttgtcccagttggggcgtggcctgctgcatacggggccgggaaagacgcctcacctgggatgacgcggaccattggacacggcctttccggccgcgggaaggaccagacgtgaatcccttcgtcaagacggcgcgcgtggctatcacctcgacgctggtggcaggctcgctcgccactgccagcctcgtgtttgcaccacttgcacaggccgattactcccccaacgactcgcccttctacgtgaaccccaacatgtcgtcggccgagtgggtgcgcaacaaccccaacgacccccgcacccccgtgatccgcgaccgcatcgcctcggtgccccagggcacctggttcgcccaccacaaccccggccagatcaccggccaggtggacgccctgatgtcggccgcccaggccgccggcaagatccccatcctggtggtgtacaacgcccccggccgcgactgcggcaaccactcgtcgggcggcgccccctcgcactcggcctaccgctcgtggatcgacgagttcgccgccggcctgaagaaccgccccgcctacatcatcgtggagcccgacctgatctcgctgatgtcgtcgtgcatgcagcacgtgcagcaggaggtgctggagaccatggcctacgccggcaaggccctgaaggccggctcgtcgcaggcccgcatctacttcgacgccggccactcggcctggcactcgcccgcccagatggcctcgtggctgcagcaggccgacatctcgaactcggcccacggcatcgccaccaacacctcgaactaccgctggaccgccgacgaggtggcctacgccaaggccgtgctgtcggccatcggcaacccctcgctgcgcgccgtgatcgacacctcgcgcaacggcaacggccccgccggcaacgagtggtgcgacccctcgggccgcgccatcggcaccccctcgaccaccaacaccggcgaccccatgatcgacgccttcctgtggatcaagctgcccggcgaggccgacggctgcatcgccggcgccggccagttcgtgccccaggccgcctacgagatggccatcgccgccggcggcaccaaccccaaccccaaccccaaccccacccccacccccacccccacccccacccccccccccggctcgtcgggcgcctgcaccgccacctacaccatcgccaacgagtggaacgacggcttccaggccaccgtgaccgtgaccgccaaccagaacatcaccggctggaccgtgacctggaccttcaccgacggccagaccatcaccaacgcctggaacgccgacgtgtcgacctcgggctcgtcggtgaccgcccgcaacgtgggccacaacggcaccctgtcgcagggcgcctcgaccgagttcggcttcgtgggctcgaagggcaactcgaactcggtgcccaccctgacctgcgccgcctcg

SEQ ID NO: 49 - DNA, 2604 bp, Artificial Sequence
  Polynucleotide, expression cassette
ccaccgtgagctgcacctgaatcagctgagaatgccctgaaccctcaaacccgcaccctgaacctcaaccttgcgttgaacccgaggggtgcgggttggacgcgcaccccgaccactgcaccccgcggggcgcccctgtgacgaccatggtctgattcacgcctgaaatcactccccgccggggtggagaaccacgtcaacgcggccgtggatcacatcgggcgtcgaaaaacaaccccccatttccccaaccctcaacctgatcctgcactgttgtcgggtttgctgagagccgcctaagctgccgcacgttgtcccagttggggcgtggcctgctgcatacggggccgggaaagacgcctcacctgggatgacgcggaccattggacacggcctttccggccgcgggaaggaccagacgtgaatcccttcgtcaagacggcgcgcgtggctatcacctcgacgctggtggcaggctcgctcgccactgccagcctcgtgtttgcaccacttgcacaggccgattactcccccgccggcacctacaactacggcgaggccctgcagaagtcgatcatgttctacgagttccagcgctcgggcgacctgcccgccgacaagcgcgacaactggcgcgacgactcgggcatgaaggacggctcggacgtgggcgtggacctgaccggcggctggtacgacgccggcgaccacgtgaagttcaacctgcccatgtcgtacacctcggccatgctggcctggtcgctgtacgaggacaaggacgcctacgacaagtcgggccagaccaagtacatcatggacggcatcaagtgggccaacgactacttcatcaagtgcaaccccacccccggcgtgtactactaccaggtgggcgacggcggcaaggaccactcgtggtggggccccgccgaggtgatgcagatggagcgcccctcgttcaaggtggacgcctcgaagcccggctcggccgtgtgcgcctcgaccgccgcctcgctggcctcggccgccgtggtgttcaagtcgtcggaccccacctacgccgagaagtgcatctcgcacgccaagaacctgttcgacatggccgacaaggccaagtcggacgccggctacaccgccgcctcgggctactactcgtcgtcgtcgttctacgacgacctgtcgtgggccgccgtgtggctgtacctggccaccaacgactcgacctacctggacaaggccgagtcgtacgtgcccaactggggcaaggagcagcagaccgacatcatcgcctacaagtggggccagtgctgggacgacgtgcactacggcgccgagctgctgctggccaagctgaccaacaagcagctgtacaaggactcgatcgagatgaacctggacttctggaccaccggcgtgaacggcacccgcgtgtcgtacacccccaagggcctggcctggctgttccagtggggctcgctgcgccacgccaccacccaggccttcctggccggcgtgtacgccgagtgggagggctgcaccccctcgaaggtgtcggtgtacaaggacttcctgaagtcgcagatcgactacgccctgggctcgaccggccgctcgttcgtggtgggctacggcgtgaaccccccccagcacccccaccaccgcaccgcccacggctcgtggaccgaccagatgacctcgcccacctaccaccgccacaccatctacggcgccctggtgggcggccccgacaacgccgacggctacaccgacgagatcaacaactacgtgaacaacgagatcgcctgcgactacaacgccggcttcaccggcgccctggccaagatgtacaagcactcgggcggcgaccccatccccaacttcaaggccatcgagaagatcaccaacgacgaggtgatcatcaaggccggcctgaactcgaccggccccaactacaccgagatcaaggccgtggtgtacaaccagaccggctggcccgcccgcgtgaccgacaagatctcgttcaagtacttcatggacctgtcggagatcgtggccgccggcatcgaccccctgtcgctggtgacctcgtcgaactactcggagggcaagaacaccaaggtgtcgggcgtgctgccctgggacgtgtcgaacaacgtgtactacgtgaacgtggacctgaccggcgagaacatctaccccggcggccagtcggcctgccgccgcgaggtgcagttccgcatcgccgccccccagggcaccacctactggaaccccaagaacgacttctcgtacgacggcctgcccaccacctcgaccgtgaacaccgtgaccaacatccccgtgtacgacaacggcgtgaaggtgttcggcaacgagcccgccggcggctcggagaaccccgaccccgagatcctgtacggcgacgtgaactcggacaagaacgtggacgccctggacttcgccgccctgaagaagtacctgctgggcggcacctcgtcgatcgacgtgaaggccgccgacacctacaaggacggcaacatcgacgccatcgacatggccaccctgaagaagtacctgctgggcaccatcacccagctgccccagggc

SEQ ID NO: 50 - DNA, 49 bp, Artificial Sequence
  Primer
gatcggcacgtaagaggttccaactttcaccccgaggtccacacgccgg

SEQ ID NO: 51 - DNA, 49 bp, Artificial Sequence
  Primer
ggcgcgcttgttcagggggccctcctgcagggcattcgcaggcgccatg

SEQ ID NO: 52 - DNA, 30 bp, Artificial Sequence
  Primer
ctgcaggagggccccctgaacaagcgcgcc

SEQ ID NO: 53 - DNA, 50 bp, Artificial Sequence
  Primer
ctatttcgttcatccatagttgcctgactcatgggattcagttgtaggtg

SEQ ID NO: 54 - DNA, 49 bp, Artificial Sequence
  Primer
ccacgagtcccaggtggcacgcttcgagatggcattcgcaggcgccatg

SEQ ID NO: 55 - DNA, 30 bp, Artificial Sequence
  Primer
atctcgaagcgtgccacctgggactcgtgg

SEQ ID NO: 56 - DNA, 49 bp, Artificial Sequence
  Primer
cacggcggagatgttgttgaagcggtcgatggcattcgcaggcgccatg

SEQ ID NO: 57 - DNA, 30 bp, Artificial Sequence
  Primer
atcgaccgcttcaacaacatctccgccgtg

SEQ ID NO: 58 - DNA, 49 bp, Artificial Sequence
  Primer
cttgatcgacttctcgaccaggttctccatggcattcgcaggcgccatg

SEQ ID NO: 59 - DNA, 30 bp, Artificial Sequence
  Primer
atggagaacctggtcgagaagtcgatcaag

SEQ ID NO: 60 - DNA, 49 bp, Artificial Sequence
  Primer
caggcgctcgatcttgatgttggagatgttggcattcgcaggcgccatg

SEQ ID NO: 61 - DNA, 30 bp, Artificial Sequence
  Primer
aacatctccaacatcaagatcgagcgcctg

SEQ ID NO: 62 - DNA, 49 bp, Artificial Sequence
  Primer
gccgttgcccatggggatgtagcgcaccatggcattcgcaggcgccatg

SEQ ID NO: 63 - DNA, 30 bp, Artificial Sequence
  Primer
atggtgcgctacatccccatgggcaacggc

SEQ ID NO: 64 - DNA, 49 bp, Artificial Sequence
  Primer
gatcggcacgtaagaggttccaactttcaccccaccgtgagctgcacct

SEQ ID NO: 65 - DNA, 52 bp, Artificial Sequence
  Primer
ggcgcgcttgttcagggggccctcctgcagggcctgtgcaagtggtgcaaac

SEQ ID NO: 66 - DNA, 52 bp, Artificial Sequence
  Primer
ccacgagtcccaggtggcacgcttcgagatggcctgtgcaagtggtgcaaac

SEQ ID NO: 67 - DNA, 52 bp, Artificial Sequence
  Primer
cacggcggagatgttgttgaagcggtcgatggcctgtgcaagtggtgcaaac

SEQ ID NO: 68 - DNA, 52 bp, Artificial Sequence
  Primer
cttgatcgacttctcgaccaggttctccatggcctgtgcaagtggtgcaaac

SEQ ID NO: 69 - DNA, 52 bp, Artificial Sequence
  Primer
caggcgctcgatcttgatgttggagatgttggcctgtgcaagtggtgcaaac

SEQ ID NO: 70 - DNA, 52 bp, Artificial Sequence
  Primer
gccgttgcccatggggatgtagcgcaccatggcctgtgcaagtggtgcaaac

SEQ ID NO: 71 - DNA, 50 bp, Artificial Sequence
  Primer
gatcggcacgtaagaggttccaactttcacctcggtcgggtccggacggg

SEQ ID NO: 72 - DNA, 54 bp, Artificial Sequence
  Primer
cacgcgcgccgtcttgacgaagggattcacgtagtagggctccgatgttggatg

SEQ ID NO: 73 - DNA, 43 bp, Artificial Sequence
  Primer
cggccgcgggaaggaccagacatgctcactcgcaagagagtgg

SEQ ID NO: 74 - DNA, 40 bp, Artificial Sequence
  Primer
ccaggtggcacgcttcgagatggggccggtggcggcgctg

SEQ ID NO: 75 - DNA, 55 bp, Artificial Sequence
  Primer
gttctgggcggccagctcgcgcagggtcgactcggcctgtgcaagtggtgcaaac

SEQ ID NO: 76 - DNA, 33 bp, Artificial Sequence
  Primer
gagtcgaccctgcgcgagctggccgcccagaac

SEQ ID NO: 77 - DNA, 55 bp, Artificial Sequence
  Primer
tctatttcgttcatccatagttgcctgactcgagcaggtggcgccgttcagggtg

SEQ ID NO: 78 - DNA, 55 bp, Artificial Sequence
  Primer
catgttggggttcacgtagaagggcgagtcgttggcctgtgcaagtggtgcaaac

SEQ ID NO: 79 - DNA, 33 bp, Artificial Sequence
  Primer
aacgactcgcccttctacgtgaaccccaacatg

SEQ ID NO: 80 - DNA, 55 bp, Artificial Sequence
  Primer
tctatttcgttcatccatagttgcctgactcgaggcggcgcaggtcagggtgggc

SEQ ID NO: 81 - DNA, 55 bp, Artificial Sequence
  Primer
ctgcagggcctcgccgtagttgtaggtgccggcggcctgtgcaagtggtgcaaac

SEQ ID NO: 82 - DNA, 33 bp, Artificial Sequence
  Primer
gccggcacctacaactacggcgaggccctgcag

SEQ ID NO: 83 - DNA, 51 bp, Artificial Sequence
  Primer
tctatttcgttcatccatagttgcctgactgccctggggcagctgggtgat

SEQ ID NO: 84 - DNA, 1212 bp, Artificial Sequence
  Polynucleotide encoding a Propionibacterium replication protein
tcagtccacctccaccaattcgtgttcgattccgtgctcttgcagccaccgcgccaagccagcttgaccgcccaattcgcaacttcgcagacactcgtagagcttctgctgccccaccaggcgacgccatccgtcacccgtgatcaaagcgacagtgtcagcaacagacccgacttcctcagccgcgatcacgtcgtcagattcctcaaccatgaggccgagacggtccctcaaccccgcagaccagccaatctgtctgcgtccccggctacccttctcccactcgaaccagagaccaacctctttcgccaagccgttggccgcgtcgcccaggacctcccaagtcgatcgagtcgagagcgcagaacgcgccgtcttgctctgggagttcgtcaactcgtggccgatcttcccctggaactgcgccttgctcaggtagcgcgcgaggtgatccaggccagttgctgcgctcatctgctgaacgtcttgcgcgcgagcaaggggagtcccgaggccagcagcgagcacgccgcgttcccaacggccgaacatggaccggtgcagcgccagagcgtcgccgaagtcgccgaccaggaacacgagcacatgcagatgcacatgccacccattgcgcccgtgcgtaacctcgaccacacgcacgaagccctcgaccccgtgacggagctggtcagacgtccagcccttgcccgaagtgactcgccgccaccccgaagcgacaccatcccaaacagccgtcaaggaatccttacgagagtgccgaaccgtgaacgtcatgaacgccacacgaccaccgtgcttagtccaggtctcgaccgccgcgccgagttcaaggccacgccgagccatgatcttcgcgttacacaccgggcaggcccagaccgatccgcagctctgcaacccagcgaaaccggcccggccgtcgctgcaccgcacaccaaccgaagcgaccgccgaggcggcaacacgaccgcagaacgcaacccgcttcaacgacgtatgacgccacaaccaataacggaccgaaaaccggtgtttgcgcttgtcagcagccacttcaccagcagccggcaccgcggaaggtgaaacattgttcgcatgattatctagggcgccccctagcggggccaccgcagggctcccgcccccacacccccggacgccacgacttcgcgccctggcatcgaccaccat

SEQ ID NO: 85 - DNA, 1176 bp, Artificial Sequence
  Chloramphenicol resistance gene
tcaattggcctccgccgcggtcttcgtaagcgcgcgtctggtgagaagcatgatgacgagagcgatcgctgtcagcaccgaagcgacccaaaccggcgcgagcagccccagcccggtcgcgagcccgagcgcaccaagcacgggccccgctgcagctccgatattcaatgctgcggttgcgtacgaaccgcccatcgttggcgcacccgatgctgcatacagcacacgcgtgatcagagtactgccgacgccgaacgacaggaatccctgaacgaggacgaggacgataagcgcaacgggatgagatgcgaccactgccaacacgatccagcctgtcagcaatagcggtccgccgactgcgagcacgaggccaggtcgttgatctgatagtcgtcctgcgatcgtgacgccaaggaacgatccgatgccgaacatcaccagcgcgacggacacccacgcttcggccaagcccgcggtctcggtcacgatgggtgccaggaaggtgaatgccgcaaaggtccctccgttgatcagcgctccgagtgccatggccaggatgagccgcggcgtcgccaactggctgagctcgacacggagccttggtgaggtcgcgctagtctcgctccgaccaacattgttcgtgacgccacgaatgactccaacggccgcgggaatacagaggatggcgatcgcccagaacgtcgttcgccagcccagcgctgtgccgagcagtgccccggcggggacgcccacgacggttgcgatcgtcgtgccggagagcaggatcgacagtgcacgccccttctggttcgctggcacgagggtagtggccgtgctcagtgctacggcgaggaatcctgcgtttgcgagagcgctgagcacccgggtgatgagcaggagagagaacactggtgtcatcgctccgatgacgtggcttcccgcgaacacgagaaggcaaacgatcaatgtgagccgcggtggccaacggcgagcgaatgccgccatcactggcgcgccgacgaccataccgactgcgaatgcggaggtcagcaggcccgcagtgccgaccgagacgtcaagttcggtcgcgatcgcggggagcaatcccgcgagcatgaattctgaagtgcccatgacgaagaccgccagggcaagcatgtagagggcaaaaggcat

SEQ ID NO: 86 - DNA, 54 bp, Artificial Sequence
  Chloramphenicol resistance gene
tcaagaaatggttcttcttgtcaccacggccagcgccccgggtacgccagacat

SEQ ID NO: 87 - protein, 27 aa, Artificial Sequence
  Signal peptide
MATGAAAAMFVTTFAGMAPANAKEVAS

SEQ ID NO: 88 - protein, 39 aa, Artificial Sequence
  Signal peptide
MNPFVKTARVAITSTLVAGSLATASLVFAPLAQADYSPL

SEQ ID NO: 89 - protein, 56 aa, Artificial Sequence
  Signal peptide
MASSNAAKSASRREQLRAAREREAAAARRKRIIIVTVVVVIIAALVAVIAMAASGV

SEQ ID NO: 90 - protein, 33 aa, Artificial Sequence
  Signal peptide
MLTRKRVVAAGAAATLSLTAFAGLQPASAATGP

SEQ ID NO: 91 - protein, 58 aa, Artificial Sequence
  Signal peptide
MASSNAAKSASRREQLRAAREREAAAARRKRITVVAVAVVVAAVLVAVIAMAAGGVFG

SEQ ID NO: 92 - protein, 41 aa, Artificial Sequence
  Signal peptide
MAKLLYRLGRGAAHRAWAVIICWLIVLAAAGGAYAAFHGTL

SEQ ID NO: 93 - protein, 39 aa, Artificial Sequence
  Signal peptide
MSRSGRRNRLVAFLLLTALVASTIMLGTSSAHADDGWSS

SEQ ID NO: 94 - protein, 38 aa, Artificial Sequence
  Signal peptide
MRRGVHGAVVLLLGAMMATLWVALGPGTPARAQTDQPV

SEQ ID NO: 95 - protein, 34 aa, Artificial Sequence
  Signal peptide
MRRFFSAAIAILLAATLTPALNAPMASAADQTSA

SEQ ID NO: 96 - protein, 33 aa, Artificial Sequence
  Signal peptide
MRLARRVAAVLLASVLALTVASCAGAARSAPSL

SEQ ID NO: 97 - protein, 62 aa, Artificial Sequence
  Signal peptide
MDSPSRSDATTDGEDDAMTAQSRRPGQAARFGRVPIAVLVTALLLVVGVLAATPPTAGATPG

SEQ ID NO: 98 - protein, 38 aa, Artificial Sequence
  Signal peptide
MTHVSVTRPRRIGALALAAAAAVALSACGASGMLAQSP

SEQ ID NO: 99 - protein, 37 aa, Artificial Sequence
  Signal peptide
MSSARRARRPSVFTLVVVGVVALVLVLGAAAAWQVWG

SEQ ID NO: 100 - protein, 27 aa, Artificial Sequence
  Signal peptide
MFGKRRIALILLSLLVAGSVVAAVSPS

SEQ ID NO: 101 - protein, 27 aa, Artificial Sequence
  Signal peptide
MATAAAATLVISSLGVTASAGADTVKG

SEQ ID NO: 102 - protein, 54 aa, Artificial Sequence
  Signal peptide
MIMPSSRRRPLTPTPSGVRASWTRLVGLAALPLVLVAAGCAASPGSGTSPSAGT

SEQ ID NO: 103 - protein, 65 aa, Artificial Sequence
  Signal peptide
MNPVSPRNSAPSHSASSQGSAAPHVLSTTLSRRRLLSMAALGAGAVALAACAGPSTSSGASSSAA

SEQ ID NO: 104 - protein, 34 aa, Artificial Sequence
  Signal peptide
MTFRLRARPVIAALSVLALAACAQPPSARHTETG

SEQ ID NO: 105 - protein, 35 aa, Artificial Sequence
  Signal peptide
MKLRPLVLVVGAALSVTLVAGCGGNSSGGASSSAP

SEQ ID NO: 106 - protein, 57 aa, Artificial Sequence
  Signal peptide
MTAGRPGARQRPPGAAPHRRTGGATRAALMGFLALLMVALGLTAAPQRALAQTSDSF

SEQ ID NO: 107 - protein, 56 aa, Artificial Sequence
  Signal peptide
MKSATRRPLTRWIVAFGVVLVLVIAGSVGLHASGALVSGGASTVPAGVFATPATNL

SEQ ID NO: 108 - protein, 36 aa, Artificial Sequence
  Signal peptide
MRLQRRGLLAWAGAAAAAATLSACGGKSSSSSTLSS

SEQ ID NO: 109 - protein, 54 aa, Artificial Sequence
  Signal peptide
MSPRHADDASGDRRRTGPQARLPRWLAGLLCIVLTTLSLGVVHPAVRADDATLD

SEQ ID NO: 110 - protein, 28 aa, Artificial Sequence
  Signal peptide
MKRVLIVVLALLLIIAGGLAGNAFRNAR

SEQ ID NO: 111 - protein, 53 aa, Artificial Sequence
  Signal peptide
MESIVVTPRNPHRSHRPRLRRASRLAVFGMALALVTACSASPGGAGGSTSARD

SEQ ID NO: 112 - protein, 36 aa, Artificial Sequence
  Signal peptide
MKYRPLVVLGSCALALGLVAGCSAKPAVSGAGSSAS

SEQ ID NO: 113 - protein, 51 aa, Artificial Sequence
  Signal peptide
MSMTDHSDHAASASASRHRWTRWASLALVPILLLAAGCSKSDASASPSSAS

SEQ ID NO: 114 - protein, 49 aa, Artificial Sequence
  Signal peptide
MNTMNGPQEVSVNRHPWRRRFAALGSAAAIALGGMTFVSPAEAQAAGVQ

SEQ ID NO: 115 - protein, 49 aa, Artificial Sequence
  Signal peptide
MKHAHRILALVAAAPLLLTACGSNDGSTPASTPSSSTPTTASASHSATP

SEQ ID NO: 116 - protein, 49 aa, Artificial Sequence
  Signal peptide
MSDTPRPSHDPRPKVTLRTVLIAIGVVLVVLIAGAIVVTTRNASTPDTN

SEQ ID NO: 117 - protein, 39 aa, Artificial Sequence
  Signal peptide
MSHTVRGYLRALGAPVALVFAVTACGAPQAGQASSSATT

SEQ ID NO: 118 - protein, 33 aa, Artificial Sequence
  Signal peptide
MSRRFATTLAASALSLVVLAGCSTATAGPSSGT

SEQ ID NO: 119 - protein, 46 aa, Artificial Sequence
  Signal peptide
MQASHPSRSTRWCALIAGCLGSALVLSACSAGSGGSTAPASSSRAA

SEQ ID NO: 120 - protein, 44 aa, Artificial Sequence
  Signal peptide
MKSSPTLAVAALGLCAITALAGCSSSTSGSSSSPSNTATALSMK

SEQ ID NO: 121 - protein, 39 aa, Artificial Sequence
  Signal peptide
MSLARRALTVALAGACALVALSGCGGSTSTAPASTPGSH

SEQ ID NO: 122 - protein, 45 aa, Artificial Sequence
  Signal peptide
MLAGPSKKSIPYTRTAGFILAAIPAVGLSLALAPSHASADTVSET

SEQ ID NO: 123 - protein, 44 aa, Artificial Sequence
  Signal peptide
MSSRQGDSRLYSFVILAVLSVTCGVFIAGMGIPFAALASDAAGS

SEQ ID NO: 124 - protein, 44 aa, Artificial Sequence
  Signal peptide
MADKKPGRGRKITRRMATVLVGALAGLLFVAAATTSHGSDLRPT

SEQ ID NO: 125 - protein, 44 aa, Artificial Sequence
  Signal peptide
MKATRRSVLAGSMAVAGSWLLAACSANSPSNSVSQAFATPSALT

SEQ ID NO: 126 - protein, 43 aa, Artificial Sequence
  Signal peptide
MNVISPLVPSGRSRIACLVAAALIVLGLGVPAIRAAAVPTPDQ

SEQ ID NO: 127 - protein, 42 aa, Artificial Sequence
  Signal peptide
MSRSSLRIAAAGLVAVTLLSTAACSGSSSSTSSSSAAALPSV

SEQ ID NO: 128 - protein, 42 aa, Artificial Sequence
  Signal peptide
MFISRFRRAAAVGLAAVTALSATACSGSSSSSSSSASSALPS

SEQ ID NO: 129 - protein, 43 aa, Artificial Sequence
  Signal peptide
MNFTPRTRLSRWAVGLVLGALIVPLAACSTPSSSSSASAGKLN

SEQ ID NO: 130 - protein, 41 aa, Artificial Sequence
  Signal peptide
MSSTRFTNRGTLAVVALSAALLAGGLTACSSGAGSDTSPAT

SEQ ID NO: 131 - protein, 41 aa, Artificial Sequence
  Signal peptide
MAMRARHGVVRLGLVCLTALAVFGTANVSGQVAVMAEGTDA

SEQ ID NO: 132 - protein, 29 aa, Artificial Sequence
  Signal peptide
MTRHSRLTRFLLVLLALTLGTAMSACGNQ

SEQ ID NO: 133 - protein, 41 aa, Artificial Sequence
  Signal peptide
MNLSWQKRLAALAVAATLTGCGATTASSPTTTASAQAAATS

SEQ ID NO: 134 - protein, 41 aa, Artificial Sequence
  Signal peptide
MSRIQLPRLSRIAIAAAASAALIGTSFIAPATAFAAPNTPT

SEQ ID NO: 135 - protein, 40 aa, Artificial Sequence
  Signal peptide
MGFRVGRRPLIGAVLAGSMATLVGCSTSGSGSGASSQAHN

SEQ ID NO: 136 - protein, 40 aa, Artificial Sequence
  Signal peptide
MTLRRHLAPALVAPALAAAIFLGGCAAQNPRGDASSSGAS

SEQ ID NO: 137 - protein, 40 aa, Artificial Sequence
  Signal peptide
MLKPRLVVLGLSLALAMVGCARTPPSSGSSSAHAQVKACL

SEQ ID NO: 138 - protein, 41 aa, Artificial Sequence
  Signal peptide
MTLRRAVIALIAAMSLLLAGCSGSSSSSKSSASASGGAAAG

SEQ ID NO: 139 - protein, 40 aa, Artificial Sequence
  Signal peptide
MSVSLSRIACVGALATALVLSGCSGSSGSGAKVASDCKPA

SEQ ID NO: 140 - protein, 39 aa, Artificial Sequence
  Signal peptide
MSRRRFLLGVSAVVGASALGASALAGCSNIASAGTRAGQ

SEQ ID NO: 141 - protein, 39 aa, Artificial Sequence
  Signal peptide
MRSTTTKAFAGVAVLALALAGCGSNSGSSTKSADSNAKL

SEQ ID NO: 142 - protein, 38 aa, Artificial Sequence
  Signal peptide
MNHVSLKSRILVAALAAGMLGLSACSSEGPASSASSSG

SEQ ID NO: 143 - protein, 39 aa, Artificial Sequence
  Signal peptide
MKTRVRSRKPAAGLAGIALFASGLSLMSTVASRADSGLP

SEQ ID NO: 144 - protein, 38 aa, Artificial Sequence
  Signal peptide
MAERTARRLTRRSLLAIGAVGSLTALAACAGATTPFVS

SEQ ID NO: 145 - protein, 56 aa, Artificial Sequence
  Signal peptide
MADRKKSRKPRRALAPSASQRHGPHRTKKKASRARRVVTAIAITLATLMIVGVLGS

SEQ ID NO: 146 - protein, 37 aa, Artificial Sequence
  Signal peptide
MLTRRSIARRFLTGAAVVVGVAVVTIFVVDALVSPAI

SEQ ID NO: 147 - protein, 38 aa, Artificial Sequence
  Signal peptide
MHRCPLWVKYLLLVAVGVVPFFAKNVPLSLAAFAVAAV

SEQ ID NO: 148 - protein, 38 aa, Artificial Sequence
  Signal peptide
MKKLLAGLLIVLIAVAGFAGGNLLRQHTMLADGASYRF

SEQ ID NO: 149 - protein, 38 aa, Artificial Sequence
  Signal peptide
MTRNTRTAIVAALCFVLLAGAVTLIPIPFVAWSPGVTY

SEQ ID NO: 150 - protein, 37 aa, Artificial Sequence
  Signal peptide
MNRSVYRTLLRIVGIVLALVGVAAVFGGVFAHNNVTD

SEQ ID NO: 151 - protein, 37 aa, Artificial Sequence
  Signal peptide
MSKTLSRIASVASVAALAGSITVIAGQNASADSVNWD

SEQ ID NO: 152 - protein, 34 aa, Artificial Sequence
  Signal peptide
MRRAVSAVLAAVVGLGMVACGPNKVNSAVGDTIK

SEQ ID NO: 153 - protein, 36 aa, Artificial Sequence
  Signal peptide
MSRRSLLVRIGVPAAALAMFAGCSPSPATAIELDGA

SEQ ID NO: 154 - protein, 34 aa, Artificial Sequence
  Signal peptide
MPTRRSFLFAALTVALVGCAPSPVVNAPRARGSV

SEQ ID NO: 155 - protein, 35 aa, Artificial Sequence
  Signal peptide
MRRRVSHPLRVILVGMVVLACLVLTGSWALVHGAA

SEQ ID NO: 156 - protein, 35 aa, Artificial Sequence
  Signal peptide
MLRQPRQLISAGIAILLGVAFVAATFVFSASLNAG

SEQ ID NO: 157 - protein, 35 aa, Artificial Sequence
  Signal peptide
MTHLPSRFSRRAVLTGAGVAVAGGGAAWAAERFLI

SEQ ID NO: 158 - protein, 29 aa, Artificial Sequence
  Signal peptide
MRRSRCAAAVFSIALAVGATLGAVTVFVS

SEQ ID NO: 159 - protein, 34 aa, Artificial Sequence
  Signal peptide
MAHHRSAGWLALAAFVAASVLVGCDPPATTGSPS

SEQ ID NO: 160 - protein, 34 aa, Artificial Sequence
  Signal peptide
MASRALETLSRLTALVVGFVAMPTPAVRDDHGAR

SEQ ID NO: 161 - protein, 34 aa, Artificial Sequence
  Signal peptide
MSTGRMKFIKLAVPVIVACCLTPMAALADVGSPG

SEQ ID NO: 162 - protein, 34 aa, Artificial Sequence
  Signal peptide
MKRIPVAIVGCAVALVAGSAVAGSAARAARLDAV

SEQ ID NO: 163 - protein, 34 aa, Artificial Sequence
  Signal peptide
MRLPRRAVLGAIGLATLAACSRNDPIGASSSASR

SEQ ID NO: 164 - protein, 34 aa, Artificial Sequence
  Signal peptide
MTSGHRDKMCDVSAYLMLLAPSANHVYAAETATL

SEQ ID NO: 165 - protein, 33 aa, Artificial Sequence
  Signal peptide
MRKLLKLIAALGAFALALTGCSGGGSGSDSGSS

SEQ ID NO: 166 - protein, 33 aa, Artificial Sequence
  Signal peptide
MTWARHFWLKVVSAVWVLVAMGSTAVAALTITV

SEQ ID NO: 167 - protein, 32 aa, Artificial Sequence
  Signal peptide
MIALIALVLVVAGTALVVHSLSTSRDAPRDMA

SEQ ID NO: 168 - protein, 32 aa, Artificial Sequence
  Signal peptide
MRGSTKMIIVGAVLVVAIMVAGEVWGAGSSSG

SEQ ID NO: 169 - protein, 21 aa, Artificial Sequence
  Signal peptide
MSRLLPRLAAPVVAAPASVHR

SEQ ID NO: 170 - protein, 74 aa, Artificial Sequence
  Signal peptide
MMHSRRRLVYKWVTITVLMLGLAVAIDVMIGDRRSDIEYLMWSVTAAAIGYAGSFGHDWWVRAGFSGRHHPLAG

SEQ ID NO: 171 - protein, 31 aa, Artificial Sequence
  Signal peptide
MKITRALAATATLTALLVGTGVAGAAADVTW

SEQ ID NO: 172 - protein, 30 aa, Artificial Sequence
  Signal peptide
MTKPWSRVVVAVLALLLVAGLVAAGVTGLF

SEQ ID NO: 173 - protein, 30 aa, Artificial Sequence
  Signal peptide
MRRVLSSVTSLLVLLALVPGGAIALVHWGR

SEQ ID NO: 174 - protein, 30 aa, Artificial Sequence
  Signal peptide
MWNAVLVLTCLSAGLLVGTAVQARLRAPGC

SEQ ID NO: 175 - protein, 29 aa, Artificial Sequence
  Signal peptide
MRRRTTIAALAAVLSFSPLAAQAAPASAD

SEQ ID NO: 176 - protein, 27 aa, Artificial Sequence
  Signal peptide
MKRRTLLGTLGIMGLSVPLAACSSKSS

SEQ ID NO: 177 - protein, 27 aa, Artificial Sequence
  Signal peptide
MTVAGLVTLMGLTGCVPFACSAIGWSN

SEQ ID NO: 178 - protein, 27 aa, Artificial Sequence
  Signal peptide
MAIVLLTCATGSPGVTTSALALTLCWP

SEQ ID NO: 179 - protein, 23 aa, Artificial Sequence
  Signal peptide
MALSLVAAGCSSTGATASVPTRS

SEQ ID NO: 180 - DNA, 66 bp, Artificial Sequence
  Polynucleotide encoding a signal peptide
gtggccacaggtgctgcggccgccatgtttgtgacgacgtttgcgggcatggcgcctgcgaatgcc

SEQ ID NO: 181 - DNA, 114 bp, Artificial Sequence
  Polynucleotide encoding a signal peptide
gtgaatcccttcgtcaagacggcgcgcgtggctatcacctcgacgctggtggcaggctcgctcgccactgccagcctcgtgtttgcaccacttgcacaggccgattactccccc

SEQ ID NO: 182 - DNA, 168 bp, Artificial Sequence
  Polynucleotide encoding a signal peptide
atggcctcatccaacgccgccaagtcggcgagtcgccgcgagcagctgcgcgccgcccgtgaacgcgaggctgcggccgcccgccgcaagcgcatcatcatcgtgaccgtcgtggtcgtcatcatcgccgccctggttgccgtgatcgcgatggccgcctccggcgtc

SEQ ID NO: 183 - DNA, 99 bp, Artificial Sequence
  Polynucleotide encoding a signal peptide
atgctcactcgcaagagagtggttgcagcgggagctgccgccaccctgtccctcacggcgtttgccgggttgcagcccgccagcgccgccaccggcccc

SEQ ID NO: 184 - DNA, 174 bp, Artificial Sequence
  Polynucleotide encoding a signal peptide
atggcctcatccaacgccgccaagtcggcgagtcgccgcgagcagctgcgcgccgcccgtgaacgcgaggctgcggccgcccgccgcaagcgcatcaccgtcgtcgccgtggcggtggtcgttgcagcggtgttggtcgccgtgatcgccatggccgccggtggtgtgttcggg

SEQ ID NO: 185 - DNA, 123 bp, Artificial Sequence
  Polynucleotide encoding a signal peptide
atggcgaagttgttgtaccggttgggccgaggggccgcacaccgggcctgggcggtgatcatctgctggctaatcgtgctggccgcagccggtggcgcctatgccgcgttccacggcaccctg

SEQ ID NO: 186 - DNA, 117 bp, Artificial Sequence
  Polynucleotide encoding a signal peptide
gtgagtcggtcgggacggcgcaaccggttggttgcgttcctgttgctgacggccctcgtggcgtccaccatcatgctgggcacatcgtcagcgcacgccgacgacggctggtcgagc

SEQ ID NO: 187 - DNA, 114 bp, Artificial Sequence
  Polynucleotide encoding a signal peptide
atgcgccgcggggttcacggtgccgtggtgctactgctcggcgccatgatggcgaccctgtgggtggccctcggccccggcacgccggcccgtgcccagaccgaccaaccggtg

SEQ ID NO: 188 - DNA, 102 bp, Artificial Sequence
  Polynucleotide encoding a signal peptide
atgcgtcgattcttcagcgctgccatcgcaatcctgctggcagccaccctcacaccagcgctcaacgcgcccatggcgagtgctgcggaccagacgtccgcg

SEQ ID NO: 189 - DNA, 99 bp, Artificial Sequence
  Polynucleotide encoding a signal peptide
atgaggctcgcccgcagggtggcggcagtgttgctggcttcggtgttggcgttgaccgtggcgtcctgcgcaggtgcggcacggtcggcgcccagcctg

(19) United States
(12) Patent Application Publication
(10) Pub. No.: US 2019/0169586 A1
(43) Pub. Date: Jun. 6, 2019

(54) LACTIC ACID-UTILIZING BACTERIA GENETICALLY MODIFIED TO SECRETE POLYSACCHARIDE-DEGRADING ENZYMES

(71) Applicant: 3PLW LTD., Netanya (IL)

(72) Inventors: Tal SHAPIRA, Even Yehuda (IL); Amir ORANIM, Even Yehuda (IL)

(73) Assignee: 3PLW LTD., Netanya (IL)

(21) Appl. No.: 16/068,410

(22) PCT Filed: Jan. 11, 2017
(86) PCT No.: PCT/IL2017/050031

§ 371 Date: Jul. 6, 2018

Related U.S. Application Data
(60) Provisional application No. 62/276,985, filed on Jan. 11, 2016.

(51) Int. Cl. C12N 9/42; C12N 9/24; C12N 9/26; C12P 19/14; C12P 7/56; C02F 3/34

(52) U.S. Cl. CPC C12N 9/2437; C12N 9/2477; C12N 9/2411; C02F 2103/32; C12P 7/56; C02F 3/34; C12P 19/14

(57) ABSTRACT

Lactic acid (LA)-utilizing bacteria are provided, genetically modified to express and optionally secrete polysaccharide-degrading enzymes, such as cellulases, hemicellulases and amylases, and uses thereof. The polysaccharide-degrading enzymes are advantageous for processing organic waste so that the organic waste can be used as a substrate in industrial fermentation processes, particularly industrial production of discrete lactic acid enantiomer(s). Vectors and constructs useful for genetically modifying the LA-utilizing bacteria are also provided.

### FIELD OF THE INVENTION

The present invention relates to dual action lactic-acid (LA)-utilizing bacteria genetically modified to secrete polysaccharide-degrading enzymes such as cellulases, hemicellulases, and amylases, useful for processing organic waste both to eliminate lactic acid present in the waste and degrade complex polysaccharides, thus providing a substrate for industrial fermentation processes producing various biochemicals, including specific lactic acid enantiomer(s).

### BACKGROUND OF THE INVENTION

Lactic acid fermentation, namely, production of lactic acid from carbohydrate sources via microbial fermentation, has been gaining interest in recent years due to the ability to use lactic acid as a building block in the manufacture of bio-plastics. Lactic acid can be polymerized to form the biodegradable and recyclable polyester polylactic acid (PLA), which is considered a potential substitute for plastics manufactured from petroleum. PLA is used in the manufacture of various products including food packaging, disposables, fibers in the textile and hygiene products industries, and more.

Production of lactic acid by fermentation bioprocesses is preferred over chemical synthesis methods for various considerations, including environmental concerns, costs and the difficulty to generate enantiomerically pure lactic acid by chemical synthesis, which is desired for most industrial applications. The conventional fermentation process is typically based on anaerobic fermentation in batch, fed-batch, continuous or semi-continuous reactors by lactic acid-producing microorganisms, e.g. bacteria of the Lactobacillus sp., which produce lactic acid as the major metabolic end product of carbohydrate fermentation (Abdel-Rahman et al. (2013) Biotechnology Advances, 31:877-902; Ghaffar et al. (2014) Journal of Radiation Research and Applied Sciences, 7(2): 222-229). For production of PLA, the lactic acid generated during the fermentation is separated from the fermentation broth by various processes, for example processes involving solvent extraction, electro-dialysis, distillation or a combination of one or more processes. The purified lactic acid is then subjected to polymerization.

Lactic acid has a chiral carbon atom and therefore exists in two enantiomeric forms, D- and L-lactic acid. In order to generate PLA that is suitable for industrial applications, the polymerization process should utilize only one enantiomer. Presence of impurities or a racemic mixture of D- and L-lactic acid results in a polymer having undesired characteristics such as low crystallinity and low melting temperature. Thus, lactic acid bacteria that produce only L-lactate enantiomer or only D-lactate enantiomer are required.

In currently available commercial processes, the carbohydrate source is typically a starch-containing renewable source such as corn and cassava root. Additional sources, such as the cellulose-rich sugarcane bagasse, have also been proposed. Typically, lactic acid bacteria can utilize reducing sugars like glucose and fructose, but do not have the ability to degrade polysaccharides like starch and cellulose. Thus, to utilize such polysaccharides the process requires adding glycolytic enzymes, typically in combination with chemical treatment, to degrade the polysaccharides and release reducing sugars.

Sakai et al., 2004 Journal of Industrial Ecology, 7(3-4): 63-74, report about a recycling system for municipal food waste that combines fermentation and chemical processes to produce poly-L-lactate (PLLA). Food waste typically includes varied ratios of reducing sugars (glucose, fructose, lactose, etc.), starch and lignocellulosic material. The process in Sakai et al. involved treatment of minced and sterilized food waste with a glucoamylase to degrade starch in the food waste into soluble glucose, L-lactic acid fermentation using an L-forming lactic acid bacterium, and purification and polymerization steps to obtain PLLA. For the particular carbohydrate source used in Sakai et al, namely, food waste, the process is complicated due to the fact that food waste contains endogenous D,L-lactic acid (e.g., from dairy products) that need to be removed in order to obtain a final pure L- or D-lactic acid. Thus, the process in Sakai et al. further included removal of endogenous D,L-lactic acid from the food waste by a Propionibacterium that consumes lactic acid as a carbon source, prior to the lactic acid fermentation step. Only 50% of the carbon content in the food waste was utilized by the method of Sakai et al. and converted to lactic acid. In addition, although food waste contains reducing sugars, starch and lignocellulose, the latter was not utilized by the method of Sakai et al., since the lactic acid bacteria cannot directly utilize this carbohydrate source.

U.S. Pat. No. 7,507,561 discloses a process for producing polylactic acid from fermentation of renewable agricultural feed-stocks comprising molasses or cane bagasse employed as starting material.

U.S. Pat. No. 8,119,376 discloses a method for the production of lactic acid or a salt thereof wherein starch is subjected to a process of simultaneous saccharification and fermentation, the method comprising saccharifying starch in a medium comprising at least a glucoamylase and simultaneously fermenting the starch using a microorganism, and optionally isolating lactic acid from the medium, characterized in that a moderately thermophilic lactic acid-producing microorganism is used. The invention further relates to a method of performing said process in the presence of a moderately thermophilic lactic acid producing microorganism, which has been adapted to have its maximum performance at the working pH.

EP 2843039 discloses a method for preparing a D-lactic acid-producing strain modified to inhibit L-lactate dehydrogenase (L-LDH) activity and to introduce D-lactate dehydrogenase (D-LDH) activity in an L-lactic acid-producing strain, a mutated D-lactic acid-producing strain prepared by the above method, and a method for producing D-lactic acid including the steps of culturing the strain and recovering D-lactic acid from the culture media.

WO 2011/098843 discloses a procedure for producing lactic acid or its salts. The procedure describes a simultaneously conducted saccharification of starch from a starch material and fermentation of sugars thereof into lactic acid by selected bacterium that produces amylolytic enzymes.

Morais et al., 2013 Applied and Environmental Microbiology, 79(17): 5242-5249, report about the introduction of genes coding for a cellulase or a xylanase into Lactobacillus plantarum, and establishment of a two-strain cell-based consortium secreting both cellulase and xylanase. The enzymatic activity of the cell consortium was assessed on wheat straw.

WO 2015011250 discloses vectors for producing and secreting substance of interest by bacteria and applications thereof.

WO 2015/097685 discloses lactic acid cell cultures for processing lignocellulose. The bacterial culture may comprise a biomass composition and a population of lactic acid bacteria which comprises: (i) a first population of lactic acid bacteria which has been genetically modified to express a secreted cellulase; and (ii) a second population of lactic acid bacteria which has been genetically modified to express a secreted xylanase, wherein the ratio of the first population: second population is selected such that the specific activity of cellulase: xylanase in the culture is greater than 4:1 or less than 1:4.

WO 2015/097686 discloses lactic acid bacterial cultures, cell populations and articles of manufacture comprising same for generating ethanol from lignocellulose.

Hitherto described methods of producing lactic acid from renewable sources have a number of drawbacks, such as low carbon-to-lactic acid conversion rate, complicated procedure requiring many separate steps, relatively high cost, and limited sources of carbohydrates that can be utilized. In addition, some of the methods are disadvantages for using source materials that are of high value as human food.

There is thus a need for bacteria, compositions and methods for efficient processing of a variety of carbohydrate-containing sources, such as organic wastes comprising starch-rich material, lignocellulose-rich material or combinations thereof, to obtain large amounts of soluble reducing sugars suitable for use in industrial fermentation processes.

### SUMMARY OF THE INVENTION

The present invention provides, according to some aspects, lactic acid (LA)-utilizing bacteria genetically-engineered to produce one or more polysaccharide-degrading enzyme, particularly glycoside hydrolases such as cellulases, hemicellulases and amylases. The genetically-engineered dual action LA-utilizing bacteria disclosed herein facilitate degradation of complex polysaccharides found in organic wastes, such as food and agricultural wastes, in addition to their natural ability to consume and metabolize lactic acid selectively, substantially without utilizing other reducing sugars, under certain conditions. Such genetically-engineered LA-utilizing bacteria surprisingly provide improved means for processing organic wastes, enabling saccharification of the waste together with removal of endogenous lactic acid present in the waste. The processed waste can then be used as a substrate in industrial fermentation processes, particularly in the production of specific discrete lactic acid enantiomer(s) by lactic acid-producing microorganisms.

The LA-utilizing bacteria disclosed herein are advantageous for processing a large variety of organic wastes, including various and diverse food waste as well as plant material, and man-made material, such as paper. In some embodiments, the LA-utilizing bacteria disclosed herein are particularly suitable for use with mixed food waste of commercial, industrial and municipal origin. The LA-utilizing bacteria disclosed herein enable efficient and cost-effective processing of organic waste, and according to some embodiments efficient and cost-effective production of lactic acid, characterized by increased carbon-to-lactic acid conversion rate compared to other methods. Further advantages of the LA-utilizing bacteria disclosed herein are that when used for processing waste, the process does not call for addition of external degrading enzymes to the process, as such enzymes are produced by the bacteria. Thus, by avoiding the need to add such external enzymes (i.e., adding enzymes to the vessels in which the process occurs), an advantageous process is achieved, which is much more cost efficient and is much more robust, as the risk of contamination (involved in adding an external enzyme mixture to the process) is markedly reduced. Additionally, by utilizing the LA-utilizing bacteria disclosed herein for processing waste, decreased viscosity of the processed waste is obtained, as saccharification (which is achieved by the degrading enzymes produced by the bacteria) reduces viscosity. This allows for a more efficient mixing of the waste and results in increased yield production of end products, such as discrete enantiomers of lactic acid.

According to one aspect, the present invention provides a lactic-acid (LA)-utilizing bacterium genetically modified to express and secrete one or more exogenous polysaccharide-degrading enzyme, wherein the one or more exogenous polysaccharide-degrading enzyme comprises a cellulase, a hemicellulase, an amylase or combinations thereof.

As used herein, the term “exogenous”, when referring to a polysaccharide-degrading enzyme expressed by a LA-utilizing bacterium, indicates that the polysaccharide-degrading enzyme is encoded by a foreign nucleic acid introduced into the bacterial cell. In some embodiments, the exogenous polysaccharide-degrading enzyme is encoded by a nucleic acid introduced into the bacteria and is capable of being secreted from the bacterial cell. In some embodiments, the nucleic acid may be transiently expressed in the bacteria (i.e. utilizing an expression vector introduced into the bacteria). In some embodiments, the nucleic acid may be integrated into the genome of the bacteria. In some embodiments, the exogenous polysaccharide-degrading enzyme is not naturally found in these bacteria or cannot naturally be secreted therefrom.

“Polysaccharide-degrading enzymes” as used herein refers to hydrolytic enzymes (or enzymatically-active portions thereof) that catalyze the breakdown of saccharides, including bi-saccharides, oligosaccharides, polysaccharides and glycoconjugates, selected from the group consisting of glycoside hydrolases, polysaccharide lyases and carbohydrate esterases. Each possibility represents a separate embodiment of the present invention. The polysaccharide-degrading enzymes for use with the present invention are selected from those that are active towards saccharides (such as polysaccharides) found in organic wastes, including food waste and plant material. In some embodiments, the Polysaccharide-degrading enzymes may be modified enzymes (i.e., enzymes that have been modified and are different from their corresponding wild-type enzymes). In some embodiments, the modification may include one or more mutations that result in improved activity of the enzyme. In some embodiments, the Polysaccharide-degrading enzymes are wild type (WT) enzymes.

In some embodiments, the LA-utilizing bacterium is selected from the group consisting of Propionibacterium species, Megasphaera species, Selenomonas species and Veillonella species. Each possibility represents a separate embodiment of the present invention. In some embodiments, the LA-utilizing bacterium is a Propionibacterium sp. In some particular embodiments, the LA-utilizing bacterium is Propionibacterium freudenreichii.

According to another aspect, the present invention provides a population of lactic-acid (LA)-utilizing bacteria genetically modified to express and secrete a plurality of exogenous polysaccharide-degrading enzymes comprising a cellulase, a hemicellulase, an amylase or combinations thereof, wherein the population comprises a plurality of sub-populations of LA-utilizing bacteria, wherein each sub-population is genetically modified to express and secrete a different polysaccharide-degrading enzyme.

For example, in some embodiment, there is provided a population of LA-utilizing bacteria genetically modified to express and secrete a plurality of polysaccharide-degrading enzymes, wherein the population comprises:

a first sub-population of LA-utilizing bacteria genetically modified to express and secrete a first polysaccharide-degrading enzyme;

a second sub-population of LA-utilizing bacteria genetically modified to express and secrete a second polysaccharide-degrading enzyme; and

a third sub-population of LA-utilizing bacteria genetically modified to express and secrete a third polysaccharide-degrading enzyme,

wherein the first polysaccharide-degrading enzyme is a cellulase, the second polysaccharide-degrading enzyme is a hemicellulase and the third polysaccharide-degrading enzyme is an amylase. In some embodiments, the hemicellulase is a xylanase.

In some embodiments, the sub-populations are sub-populations of the same bacterial species. In other embodiments, the sub-populations are sub-populations of different bacterial species.

In some embodiments, the LA-utilizing bacteria are selected from the group consisting of Propionibacterium species, Megasphaera species, Selenomonas species and Veillonella species. In some embodiments, the LA-utilizing bacteria are selected from Propionibacterium sp. In some particular embodiments, the Propionibacterium sp. is Propionibacterium freudenreichii.

Polysaccharide-degrading enzymes for use in accordance with the present invention may be bacterial enzymes. In some embodiments, the polysaccharide-degrading enzymes are from thermophilic bacteria. In some embodiments, the thermophilic bacteria are selected from the group consisting of Clostridium sp., Paenibacillus sp., Thermobifida fusca, Bacillus sp., Geobacillus sp., Chromohalobacter sp. and Rhodothermus marinus. Each possibility represents a separate embodiment of the present invention.

In other embodiments, the polysaccharide-degrading enzymes are from mesophilic bacteria. In some embodiments, the mesophilic bacteria are selected from the group consisting of Klebsiella sp., Cohnella sp., Streptomyces sp, Acetivibrio cellulolyticus, Ruminococcus albus; Bacillus sp. and Lactobacillus fermentum. Each possibility represents a separate embodiment of the present invention.

In additional embodiments, the polysaccharide-degrading enzymes are fungal enzymes. In some embodiments, the fungi are selected from the group consisting of Trichoderma reesei, Humicola insolens, Fusarium oxysporum, Aspergillus oryzae, Penicillium fellutanum and Thermomyces lanuginosu. Each possibility represents a separate embodiment of the present invention.

In some embodiments, the polysaccharide-degrading enzymes engineered into the LA-utilizing bacteria are selected such that they have optimal activity at the same temperature and pH range that are optimal for growth of the LA-utilizing bacteria. As used herein, temperature and pH range that are optimal for growth of the LA-utilizing bacteria indicates also temperature and pH in which the LA-utilizing bacteria selectively consumes lactic acid as a carbon source (if particular conditions are required for the selective consumption).

In other embodiments, the polysaccharide-degrading enzymes are selected such that they have optimal activity at a temperature and/or pH range that is different from the optimal temperature and pH range for growth of the LA-utilizing bacteria. For example, in some embodiments, the polysaccharide-degrading enzymes have optimal activity at a temperature higher than the optimal growth temperature of the LA-utilizing bacteria. In additional embodiments, the polysaccharide-degrading enzymes have optimal activity at a pH that is higher/lower than the optimal growth pH of the LA-utilizing bacteria. In some embodiments, the polysaccharide-degrading enzymes have optimal activity at a temperature and/or pH range in which the LA-utilizing bacteria are inactivated.

In some embodiments, the LA-utilizing bacteria can selectively consume lactic acid until the latter is substantially exhausted and only thereafter can consume (ferment) the reducing sugars.

According to an additional aspect, the present invention provides a method for processing organic waste, the method comprising contacting the organic waste with the LA-utilizing bacterium of the present invention genetically modified to express and secrete one or more exogenous polysaccharide-degrading enzyme, under conditions in which lactic acid is consumed by the LA-utilizing bacterium and which are suitable for expression and activity of said one or more polysaccharide-degrading enzyme, wherein said processing eliminates D-lactic acid, L-lactic acid or both from the organic waste and at least partially degrades the polysaccharides to release reducing sugars. In some embodiments, the processing is performed in one-vessel (one-pot process).

According to a further aspect, the present invention provides a method for processing organic waste, the method comprising contacting the organic waste with the population of LA-utilizing bacteria of the present invention genetically modified to express and secrete a plurality of polysaccharide-degrading enzymes, under conditions in which lactic acid is consumed by the LA-utilizing bacteria and which are suitable for expression and activity of said plurality of polysaccharide-degrading enzymes, wherein said processing eliminates D-lactic acid, L-lactic acid or both from the organic waste and at least partially degrades the polysaccharides to release reducing sugars. In some embodiments, the processing is performed in one-vessel (one-pot process).

As used herein, “elimination”, when referring to D-lactic acid, L-lactic or both from organic waste refers to reduction to residual amounts such that there is no interference with downstream processes of producing discrete lactic acid enantiomer(s) and subsequently polymerization to polylactic acid that is suitable for industrial applications. “Residual amounts” indicates less than about 5% lactic acid, preferably less than about 3%, more preferably less than about 1%, and even less than about 0.5% lactic acid (w/w).

Organic waste for use with the methods of the present invention comprises complex polysaccharides including starch, cellulose, hemicellulose and combinations thereof. In some embodiments, the organic waste is selected from the group consisting of food waste, municipal waste, agricultural waste, plant material and combinations thereof. Food waste in accordance with the present invention encompasses food waste of plant origin. Food waste in accordance with the present invention encompasses household food waste, commercial food waste and industrial food waste. Plant material in accordance with the present invention encompasses agricultural waste and manmade products such as paper waste.

The reducing sugars typically comprise C5 sugars (pentoses), C6 sugars (hexoses) or a combination thereof. In some embodiments, said reducing sugars comprise glucose. In some embodiments, said reducing sugars comprise xylan.

In some embodiments, elimination of lactic acid from the waste is effected concomitant with saccharification (degradation of the polysaccharides to soluble reducing sugars). According to these embodiments, the LA-utilizing bacteria are genetically modified to express and secrete polysaccharide-degrading enzymes having optimal activity at the same temperature and pH range that are optimal for growth of the LA-utilizing bacteria. According to these embodiments, the method comprises contacting the organic waste with the LA-utilizing bacteria at a temperature and pH optimal for growth of the LA-utilizing bacteria and secretion of the polysaccharide-degrading enzymes. Contacting is performed for sufficient time to eliminate lactic acid (D-, L-, or both) from the waste and obtain desired level of reducing sugars.

In other embodiments, elimination of lactic acid from the waste is effected separately from (prior to) saccharification. According to these embodiments, the LA-utilizing bacteria are genetically modified to express and secrete polysaccharide-degrading enzymes having optimal activity at a temperature and/or pH range that is different from the optimal temperature and/or pH range for growth of the LA-utilizing bacteria.

In some embodiments, the elimination of the D-lactic acid, L-lactic acid or a combination thereof present in the waste is performed concomitantly or sequentially with the degradation of the polysaccharides in the waste.

In some embodiments, the polysaccharide-degrading enzymes have optimal activity at a temperature higher than the optimal growth temperature of the LA-utilizing bacteria. According to these embodiments, the method comprises: (i) contacting the organic waste with the LA-utilizing bacteria at a first temperature, the first temperature being suitable for growth of the LA-utilizing bacteria and expression of the polysaccharide-degrading enzymes, for sufficient time to eliminate D-lactic acid, L-lactic acid or both from the waste; and (ii) increasing the temperature to a second temperature, the second temperature being optimal for activity of the polysaccharide-degrading enzymes. Typically, the LA-utilizing bacteria are inactivated at the second temperature.

In some embodiments, the polysaccharide-degrading enzymes may have optimal (or enhanced) activity at a pH that is higher/lower than the optimal (or otherwise suitable) growth pH of the LA-utilizing bacteria. According to these embodiments, the method comprises: (i) contacting the organic waste with the LA-utilizing bacteria at a first pH, the first pH being suitable for growth of the LA-utilizing bacteria and expression of the polysaccharide-degrading enzymes, for sufficient time to eliminate D-lactic acid, L-lactic acid or both from the waste; and (ii) lowering/increasing the pH to a second pH, the second pH being optimal for activity of the polysaccharide-degrading enzymes. Typically, the LA-utilizing bacteria are inactivated at the second pH.

In some embodiments, the methods further comprise determining the percentage of at least one of starch, cellulose and hemicellulose in the organic waste prior to said contacting, and selecting the LA-utilizing bacterium or the population of LA-utilizing bacteria to be contacted with the organic waste according to the determined percentage. In some embodiments, the determining further comprises determining the percentage of soluble reducing sugars.

According to another aspect, the present invention provides a method for producing discrete lactic acid enantiomer(s) from organic waste, the method comprising:

(i) processing the organic waste to eliminate D-lactic acid, L-lactic acid or a combination thereof present in the waste and at least partially degrade polysaccharides in the waste to release soluble reducing sugars, by contacting the organic waste with the LA-utilizing bacterium of the present invention, or population of LA-utilizing bacteria of the present invention, genetically modified to express and secrete polysaccharide-degrading enzymes, under conditions suitable for lactic acid consumption by the LA-utilizing bacteria and for expression and activity of the polysaccharide-degrading enzymes;

(ii) inactivating the LA-utilizing bacteria;

(iii) fermenting the soluble reducing sugars obtained in (i) with a lactic acid-producing microorganism that produces only one of D-lactic acid and L-lactic acid, to obtain discrete enantiomer(s) of lactic acid; and

(iv) recovering the discrete lactic acid enantiomer(s) from the fermentation broth.

Typically, the fermenting step is carried out under anaerobic or microaerophilic conditions. The fermenting step is typically selected from the group consisting of batch, fed-batch, continuous and semi-continuous fermentation. Each possibility represents a separate embodiment of the present invention.

In some embodiments, the polysaccharide-degrading enzymes may have optimal (or otherwise suitable) activity at a temperature and/or pH range different from the temperature/pH range optimal (or otherwise suitable) for growth of the LA-utilizing bacteria. According to these embodiments, the conditions suitable for lactic acid consumption by the LA-utilizing bacteria and expression of the polysaccharide-degrading enzymes are different from the conditions suitable for activity of the enzymes. According to these embodiments, step (i) comprises: contacting the organic waste at a first temperature/pH, the first temperature/pH being suitable for lactic acid consumption by the LA-utilizing bacteria and expression of the polysaccharide-degrading enzymes, for sufficient time to eliminate D-lactic acid, L-lactic acid or both from the waste; and adjusting the temperature/pH to a second temperature/pH, the second temperature/pH being optimal for activity of the polysaccharide-degrading enzymes. Typically the LA-utilizing bacteria are inactivated at the second temperature/pH.

In some embodiments, the lactic acid-producing microorganism is a bacterium. In some embodiments, the lactic acid-producing bacterium is a Lactobacillus species. In other embodiments, the lactic-acid-producing bacterium is a Bacillus species.

In some embodiments, the lactic acid-producing bacterium is a consortium of lactic acid bacteria of different species.

Recovering lactic acid typically includes separation of the lactic acid from the fermentation broth and purification of the lactic acid. Separation and purification of lactic acid can be performed by methods known in the art.

The method typically further comprises pretreatment of the organic waste to decrease particle size and increase surface area, and also to inactivate endogenous bacteria within the waste. The pretreatment is carried out prior to processing the waste with the LA-utilizing bacteria. In some embodiments, the organic waste undergoes shredding, mincing and sterilization prior to processing with the LA-utilizing bacteria.

Sterilization may be carried out by methods known in the art, including for example, high pressure steam, UV radiation or sonication.

In some embodiments, the method further comprises analyzing the composition of the organic waste, particularly the sugar content, prior to processing thereof. In some embodiments, the method comprises analyzing the percentage of at least one of starch, cellulose and hemicellulose in the organic waste. In additional embodiments, the method comprises analyzing the percentage of reducing sugars.

In some embodiments, the method further comprises determining the quantity of total soluble reducing sugars released by the polysaccharide-degrading enzymes prior to the fermentation step.

In some embodiments, there is provided a method for producing stereo-specific lactic acid from organic waste. As used herein, the term “stereo-specific” with respect to lactic acid relates to enantiomer(s) (or discrete enantiomer(s)) of lactic acid.

According to some embodiments, the LA-utilizing bacteria disclosed herein may further be advantageously utilized to increase the overall yield of lactic acid production from organic waste, by recycling residual lactic acid (which remained as a residual after recovery of lactic acid from fermentation broth) to be re-processed by the LA-utilizing bacteria and used to cultivate LA-producing microorganism, to thereby produce high quantities of polysaccharide-degrading enzymes for the next Lactic-Acid producing cycle.

According to yet another aspect, the present invention provides a method for producing polylactic acid from organic waste, the method comprising producing discrete lactic acid enantiomer(s) by the method described above, and further comprising polymerizing the discrete lactic acid enantiomer(s) to polylactic acid.

In some embodiments, the present invention provides a method for producing polylactic acid from organic waste, the method comprising producing stereo-specific lactic acid by the method described above, and further comprising polymerizing the stereo-specific lactic acid to polylactic acid.

According to additional aspect, there is provided an expression vector for expressing and secreting an exogenous polysaccharide-degrading enzyme by a Propionibacterium, said vector comprising:
at least one nucleotide sequence encoding for a Propionibacterium replication protein;
at least one Propionibacterium origin of replication nucleotide sequence; and an
at least one expression cassette comprising:
at least one promoter nucleic acid sequence, which is operably linked to:
   at least one nucleic acid sequence encoding for a Propionibacterium signal peptide (SP), which is translationally fused to:
   at least one nucleic acid sequence encoding a polysaccharide degrading enzyme, selected from cellulase, hemicellulase, and amylase.

In some embodiments, the nucleotide sequence encoding for a Propionibacterium replication protein comprises the nucleic acid sequence of SEQ ID NO: 84. In some embodiments, the promoter nucleic acid sequence may be selected from the group consisting of SEQ ID NOs: 27-32 and homologs thereof. In some embodiments, the SP amino acid sequence may be selected from the group consisting of sequence of SEQ ID NOs: 87-179. In some embodiments, the polysaccharide degrading enzyme sequence may be selected from the group consisting of SEQ ID NOs: 14-26, and homologs thereof. In some embodiments, the expression cassette may comprise or consist of a nucleic acid sequence as denoted by any one of SEQ ID NOs: 33-49, and/or homologs thereof.

In further embodiments, there is provided a Propionibacterium lactic-acid (LA)-utilizing bacteria comprising an expression vector.

In some embodiments, there is provided an expression cassette comprising or consisting of sequence as denoted by any one of SEQ ID NOs: 33-49, and/or homologs thereof. In further embodiments, there is provided a propionibacterium lactic-acid (LA)-utilizing bacteria comprising the expression cassette.

These and further aspects and features of the present invention will become apparent from the detailed description, examples and claims which follow.

### BRIEF DESCRIPTION OF THE DRAWINGS

Exemplary embodiments are illustrated in referenced figures. Dimensions of components and features shown in the figures are generally chosen for convenience and clarity of presentation and are not necessarily shown to scale. The figures are listed below.

FIG. 1—A schematic illustration of a fermentation system, for producing optically pure Lactic Acid and recycling of residual lactate waste, according to some embodiments;

FIG. 2A—A schematic illustration of a vector; according to some embodiments;

FIG. 2B—A schematic illustration of an expression vector, according to some embodiments;

FIG. 3—A pictogram of a control Lugol assay—an agar plate containing YEL media and 0.5% starch after being exposed to Lugol solution. All the areas containing starch appear in black. A circle of degraded starch (by glucoamylase enzyme) appears colorless.

### DETAILED DESCRIPTION OF THE INVENTION

The present invention discloses for the first time LA-utilizing bacteria genetically modified to produce various exogenous polysaccharide-degrading enzymes, particularly enzymes capable of degrading complex polysaccharides present in organic waste such as food waste and lignocellulosic waste. The LA-utilizing bacteria disclosed herein are particularly advantageous for processing various sources of organic waste, prior to and for using the waste as a substrate for production of discrete enantiomer(s) of lactic acid. Such genetically-engineered LA-utilizing bacteria surprisingly provides improved means for processing organic wastes, enabling removal of endogenous lactic acid present in the waste together with saccharification of the waste. The novel dual action bacteria of this invention thus enables carrying out two stages of the process together in a “one-vessel” (“one-pot”) process. The “one-vessel process” enables using one vessel (such as, reactor, fermenter or other suitable operating unit), instead of two, thus leading to a more economical industrial process. This makes possible carrying out the two stages concomitantly in one reactor (one vessel). As the two stages may sometimes have different optimal reaction temperatures and pH, it may be recommendable sometimes to carry out the one-pot process in a sequential manner, first the removal of endogenous lactic acid present in the waste (performed at its optimal temperature and pH), followed by saccharification of the waste in the same reactor but at its own optimal temperature and pH.

### Genetically-Modified Lactic Acid (LA)-Utilizing Bacteria

LA-utilizing bacteria are engineered according to the present invention to produce one or more exogenous polysaccharide-degrading enzyme. As used herein “produce” with respect to the exogenous polysaccharide-degrading enzymes indicates expression (generation of the protein within the cells) and optionally secretion. In some embodiments, the one or more exogenous polysaccharide-degrading enzyme is engineered into the bacteria as a secreted enzyme. According to these embodiments, the LA-utilizing bacteria are genetically modified to express and secrete one or more exogenous polysaccharide-degrading enzyme. In other embodiments, the one or more exogenous polysaccharide-degrading enzyme is engineered into the bacteria as a non-secreted enzyme, to be active towards polysaccharides in the organic waste only after lysis of the LA-utilizing bacterial cells. According to these embodiments, the LA-utilizing bacteria are genetically modified to express one or more exogenous polysaccharide-degrading enzyme.

LA-utilizing bacteria for genetic modification according to the present invention may be selected from Propionibacterium species, Megasphaera species, Selenomonas species and Veillonella species. Each possibility represents a separate embodiment of the present invention.

A suitable LA-utilizing bacterium for genetic modification according to the present invention is typically characterized by defined ranges of temperature and/or pH in which it consumes lactic acid selectively as a carbon source. As used herein, “selective” with respect to consumption of lactic acid as a carbon source by a LA-utilizing bacterium indicates consumption of lactic acid by the bacterium without consuming reducing sugars available in its environment, or consuming available reducing sugars at a very slow rate such that insignificant levels are consumed over a period of several hours. Thus, when contacted with an organic waste under conditions in which it selectively consumes lactic acid, the LA-utilizing bacterium eliminates lactic acid from the waste substantially without affecting the quantity of reducing sugars present in the waste. Alternatively, a suitable LA-utilizing bacterium consumes lactic acid selectively as a carbon source regardless of specific conditions and presence of reducing sugars in its environment.

In some particular embodiments, the LA-utilizing bacterium is Propionibacterium freudenreichii. P. freudenreichii is characterized by selective consumption of lactic acid as a carbon source under certain conditions, such as, acidic conditions (optimally at pH in the range of pH=5.5-6.5, such as, for example, pH=5.5).

LA-utilizing bacteria for genetic modification according to the present invention may be bacteria that consume only one of the enantiomer(s) of lactic acid, or bacteria that consume both.

In some embodiments, the genetically modified LA-utilizing bacteria of the present invention typically comprise an expression vector comprising a polynucleotide sequence encoding a polysaccharide-degrading enzyme. In some embodiments, the expression vector comprises a polynucleotide sequence encoding a polysaccharide-degrading enzyme and a signal peptide fused in frame, such that upon transcription and translation of the polynucleotide sequence a secreted polysaccharide-degrading enzyme is generated. In some embodiments, the expression vector further comprises a suitable promoter of choice. In some embodiments, the genetically modified LA-utilizing bacteria of the present invention comprise a polynucleotide sequence encoding a polysaccharide-degrading enzyme integrated into its genome. In some embodiments, the genetically modified LA-utilizing bacteria of the present invention comprise a polynucleotide sequence encoding a polysaccharide-degrading enzyme and a signal peptide fused in frame, integrated into its genome.

In some embodiments, the terms “genetically modified”, “genetically-engineered” and “recombinant” may interchangeably be used. Thus, a genetically modified LA-utilizing bacteria is also referred to herein as a recombinant bacteria or recombinant LA-utilizing bacteria.

Genetic modification of the LA-utilizing bacteria can be performed using expression vectors, signal peptides and methods known in the art for LA-utilizing bacteria, described for example in Kiatpapan and Murooka (2001) Appl Microbiol Biotechnol., 56(1-2):144-9; Brede et al., (2005) Appl Environ Microbiol., 71(12):8077-84; Mukdsi et al., (2014) Appl Environ Microbiol., 80(2): 751-756; and WO 2015/011250.

As referred to herein, the terms “nucleic acid”, “nucleic acid sequence”, “polynucleotide”, “nucleotide” and “nucleotide sequence” may interchangeably be used. The terms are directed to polymers of deoxyribonucleotides (DNA), ribonucleotides (RNA), and modified forms thereof in the form of a separate fragment or as a component of a larger construct. The terms further include oligonucleotides composed of naturally occurring bases, sugars, and covalent internucleoside linkages, as well as oligonucleotides having non-naturally occurring portions, which function similarly to respective naturally occurring portions. A DNA may include, for example, genomic DNA, plasmid DNA, recombinant DNA or complementary DNA (cDNA), An RNA may include, for example, messenger RNA (mRNA), ribosomal RNA (rRNA) or transfer RNA (tRNA). In some embodiments, the nucleic acid sequence may be a coding sequence (i.e., a sequence that can encode for an end product in the cell, such as, a protein or a peptide). In some embodiments, the nucleic acid sequence may be a regulatory sequence (such as, for example, a promoter).

The terms “polypeptide,” “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The term “amino acid sequence” relates to a sequence composed of any one of naturally occurring amino acids, amino acids that have been chemically modified, or synthetic amino acids. The term relates to peptides and proteins, as well as fragments, analogs, derivatives and combinations of peptides and proteins.

In some embodiments, a sequence (such as, nucleic acid sequence and amino acid sequence) that is “similar” or “homologous” to a reference sequence refers herein to percent identity between the sequences. The percent identity may be at least 70%, at least 80%, at least 85%, at least 90%, at least 95%, at least 98% or at least 99%. The percent identity can be distributed randomly over the entire length of the sequences. Accordingly, homologous sequences can include, for example, variations related to mutations (such as, truncations, substitutions, deletions and/or additions of at least one amino acid or at least one nucleotide) Similar sequences can also include variations related to codon usage and degeneration of the genetic code. For example, a sequence is said to be homologous to another sequence if the homology (identity/similarity) is at least 80% over the entire length of the sequences.

The term “construct”, as used herein, refers to an artificially assembled or isolated nucleic acid molecule which may include one or more nucleic acid sequences, wherein the nucleic acid sequences may include coding sequences (that is, sequence which encodes an end product, such as, Polysaccharide-degrading enzymes), regulatory sequences (such as, promoters), non-coding sequences, or any combination thereof. The term construct includes, for example, plasmids and vectors but should not be seen as being limited thereto.

“Expression vector” and “recombinant vector” may interchangeably be used and refer to constructs that have the ability to express heterologous (exogenous) nucleic acid in the bacterial cell. In some embodiments, the expression vector can autonomously replicate in the cell. In some embodiments, the nucleic acid may be integrated into the genome of the bacterial cell.

As used herein, the terms “introducing” and “transformation” may interchangeably be used and refer to the transfer of molecules, such as, for example, nucleic acids, polynucleotide molecules, vectors, and the like into the bacterial cell(s), and more specifically into the interior of a membrane-enclosed space of a target cell(s). The molecules can be “introduced” into the target cell(s) by any means known to those of skill in the art, for example as taught by Sambrook et al. Molecular Cloning: A Laboratory Manual, Cold Spring Harbor Laboratory Press, New York (2001).

The term “promoter” is directed to a regulatory DNA sequence which can control or direct the transcription of a coding sequence to produce an mRNA in the cell. Usually, the promoter is located in the 5′ region (that is, precedes, located upstream) of the coding sequence. Promoters may be derived in their entirety from a native source, or be composed of different elements derived from different promoters found in nature, or even comprise synthetic nucleotide segments. Promoters can be constitutive (i.e. promoter activation is not regulated by an inducing agent and hence rate of transcription is constant), or inducible (i.e., promoter activation is regulated by an inducing agent). In some embodiments, various types and sequences of promoters may be utilized in the expression vectors of the current invention. In some embodiments, the promoters may be derived from various sources, species and organisms. In some embodiments, a combination of specific promoters and signal peptide sequences may be utilized in the expression vectors of the current invention.

In some exemplary embodiments, the promoter may be selected from, but not limited to a promoter having a nucleotide sequence as denoted by any one of SEQ ID NOs: 27-32, and/or homologs thereof. Each possibility is a separate embodiment.

In some embodiments, the promoter sequence comprises or consists of a nucleotide sequence as denoted by any one of SEQ ID NOs: 27-32, and/or homologs thereof. Each possibility is a separate embodiment.

In some exemplary embodiments, the promoter sequence comprises or consists of a nucleic acid sequence as denoted by SEQ ID NO. 31, or homologs thereof. In some exemplary embodiments, the promoter sequence comprises or consists of a nucleic acid sequence as denoted by SEQ ID NO. 27, or homologs thereof. In some exemplary embodiments, the promoter sequence comprises or consists of a nucleic acid sequence as denoted by SEQ ID NO. 28, or homologs thereof.

The terms “signal peptide” and “SP” refer to a short (usually 5-30 amino acids long) peptide that can be present at the N-terminus of a protein and direct the protein to be secreted from the bacterial cell. In some embodiments, the secretion is into the extracellular medium. In some embodiments, the SP sequence may be obtained from a natural source (as is, or modified). In some embodiments, the SP sequence may be designed in-silico. In some exemplary embodiments, the SP sequence is from Propionibacterium bacterial source. In some embodiments, the SP sequence is homologous to a Propionibacterium signal peptide. In some embodiments, the nucleic acid sequence encoding for the SP amino acid sequence may be included in an expression vector, such that it is in-frame with the sequence encoding the protein to be secreted. In some embodiments, amino acid sequences that are at least 60%, at least 70%, at least 80%, at least 90%, at least 95% homologous to the amino acid sequences of the SP disclosed in the current disclosure are also functional and are encompassed herein. In some embodiments, nucleic acid sequences that are at least 60%, at least 70%, at least 80%, at least 90%, at least 95% homologous to the nucleic acid sequences encoding for the SP disclosed in the current disclosure are also encompassed herein.

In some exemplary embodiments, the SP may be selected from, but not limited to an SP having an amino acid sequence as denoted by any one of SEQ ID NOs: 87-179 and/or homologs thereof. Each possibility is a separate embodiment.

In some embodiments, the nucleotide sequence encoding for a SP may be selected from a nucleic acid encoding for amino acid sequence as denoted by any one of SEQ ID NOs: 87-179, and/or homologs thereof. Each possibility is a separate embodiment.

In some embodiments, the nucleotide sequence encoding for a SP may be selected from a nucleic acid as denoted by any one of the nucleic acids as denoted by SEQ ID NOs: 180-189 and/or homologs thereof. Each possibility is a separate embodiment.

In some exemplary embodiments, the SP comprises or consists of an amino acid sequence as denoted by SEQ ID NO. 87, or homologs thereof. In some exemplary embodiments, the SP comprises or consists of an amino acid sequence as denoted by SEQ ID NO. 88, or homologs thereof. In some exemplary embodiments, a nucleic acid encoding for a SP comprises or consists of the nucleic acid sequence as denoted by SEQ ID NO. 180, or homologs thereof. In some exemplary embodiments, a nucleic acid encoding for a SP comprises or consists of the nucleic acid sequence as denoted by SEQ ID NO. 181, or homologs thereof.

In some embodiments, genetic modification of the LA-utilizing bacteria can be performed using suitable expression vectors (including suitable coding sequences for the proteins of interest (for example, polysaccharide degrading enzymes), suitable signal peptide sequences (if used) and/or suitable promoters), and methods known in the art for LA-utilizing bacteria, described, for example in Kiatpapan and Murooka (2001) Appl Microbiol Biotechnol., 56(1-2):144-9; Brede et al., (2005) Appl Environ Microbiol., 71(12):8077-84; Mukdsi et at, (2014) Appl Environ Microbiol., 80(2): 751-756; and WO 2015/011250. In some embodiments, the expression vectors allow integration of the protein of interest (and SP, if used) to the genome of the bacteria.

In some embodiments, when determining the various nucleic acid sequences utilized to genetically engineer the LA-utilizing bacteria, suitable codon usage may be applied, to allow optimal expression and/or function of the polysaccharide degrading enzymes and/or SP in the bacterial cells.

According to some embodiments, there is provided an expression vector for expressing a polysaccharide degrading enzyme in a bacterial cell, the vector comprising an expression cassette (also referred to herein as “insert”), comprising at least one suitable promoter nucleic acid sequence, operably linked to at least one nucleic acid sequence encoding for a signal peptide (SP), which is translationally fused to least one nucleic acid sequence encoding a polysaccharide degrading enzyme, selected from cellulase, hemicellulase and amylase.

In some embodiments, the expression vector comprises a plasmid backbone, one or more suitable origin of replication (Ori) sequences, one or more antibiotic resistance gene sequences and one or more expression cassettes. In some embodiments, an expression vector without an expression cassette (insert) is referred to herein as a “shuttle vector”. In some embodiments, the shuttle vector is capable of being replicated in various bacterial cell types.

In some embodiments, the expression vector is suitable for expression of an exogenous polysaccharide-degrading enzyme in Propionibacterium. In some embodiments, the Propionibacterium is Propionibacterium freudenreichii. In some embodiments, an expression vector suitable for expression of an exogenous polysaccharide degrading enzyme in Propionibacterium comprises a nucleic acid sequence encoding for a Propionibacterium replication protein.

In some exemplary embodiments, the expression vector is pOWR3 vector, as further exemplified below herein. In some embodiments, such expression vector comprises a nucleic acid sequence encoding for a Propionibacterium replication protein (such as denoted by SEQ ID NO: 84). In some embodiments, such expression vector further comprises one or more chloramphenicol resistance genes (such as, cmx(A) and cml(A) from Corynebacterium striatum pT10 plasmid). In some embodiments, such expression vector further comprises a Propionibacterium origin of replication (Ori) sequence.

In some embodiments, there is provided an expression vector for expressing and secreting an exogenous polysaccharide-degrading enzyme by a Propionibacterium, said vector comprising:
at least one nucleotide sequence encoding for a Propionibacterium replication protein;
at least one Propionibacterium origin of replication nucleotide sequence; and an at least one expression cassette comprising:
   at least one promoter nucleic acid sequence, which is operably linked to: at least one nucleic acid sequence encoding for a Propionibacterium signal peptide (SP) (or homologs thereof), which is translationally fused to at least one nucleic acid sequence encoding a polysaccharide degrading enzyme, selected from cellulase, hemicellulase, and amylase.

In some embodiments, as further exemplified herein, an expression vector for expressing an exogenous polysaccharide degrading enzyme in Propionibacterium is advantageous as it can be replicated both in e. coli (either as is, or without an expression cassette), and in Propionibacterium. Further, such vector is advantageous as it allows, due to its structure and compact size (about 5.6 kb, (without expression cassette)) improved expression (such as, high yield and quality) of polysaccharide-degrading enzymes in the bacteria. Further, the expressed polysaccharide degrading enzymes, are capable of being successfully secreted from the bacterial cells and be functional in degrading polysaccharides in the substrate. Further, being smaller than other vectors used for expression in P. freudenreichii is advantageous as it makes it easier to transform to bacterial cells and easier to use for cloning by the restriction free method. Moreover, the cloning region for inserting an expression cassette is bordered (upstream and downstream) by termination signals, which can prevent polar effect interruptions and stabilizes the mRNA transcript of the polysaccharide degrading enzyme produced in the bacterial cell, to thereby results in improved expression of the proteins in the cell.

In some embodiments, the expression vector may include more than one expression cassettes, each comprising the same or different promoter, same or different SP sequence and a different polysaccharide degrading enzyme sequence.

In some exemplary embodiments, an expression cassette may comprise or consist of a nucleotide sequence as denoted by any one of SEQ ID NOs: 33-49, and/or homologs thereof. Each possibility is a separate embodiment.

In some embodiments, there is provided an expression cassette, said expression cassette comprise or consist of a nucleotide sequence as denoted by any one of SEQ ID NOs: 33-49 and/or homologs thereof. Each possibility is a separate embodiment.

In some exemplary embodiments, the cassette comprise or consist of a nucleotide sequence as denoted by SEQ ID NO: 43.

In some embodiments, when said expression cassette is expressed in the target bacterial cell, under the control of the promoter sequence of the expression cassette, the signal peptide is translationally linked in-frame to the polysaccharide degrading enzyme. Thus, a chimeric protein which includes the signal peptide sequence and the polysaccharide degrading enzyme sequence is expressed in the bacterial cell and is capable of being secreted therefrom.

In some embodiments, there is provided a LA-utilizing bacteria comprising one or more expression vector(s) as disclosed herein.

In some embodiments, there is provided a LA-utilizing bacteria comprising one or more expression cassette(s), as disclosed herein.

In some embodiments, there is provided a Propionibacterium comprising one or more expression vectors wherein the expression vector comprises: at least one nucleotide sequence encoding for a Propionibacterium replication protein;
at least one Propionibacterium origin of replication nucleotide sequence; and an at least one expression cassette comprising:
   at least one promoter nucleic acid sequence, which is operably linked to: at least one nucleic acid sequence encoding for a Propionibacterium signal peptide (SP), which is translationally fused to at least one nucleic acid sequence encoding a polysaccharide degrading enzyme, selected from cellulase, hemicellulase, and amylase.

In some embodiments, there is provided an isolated nucleic acid molecule comprising or consisting of a sequence selected from the group consisting SEQ ID NOs: 33-49, and homologs thereof.

In some embodiments, the polysaccharide degrading enzyme sequence is selected from the group consisting of SEQ ID NOs: 14-26, and homologs thereof.

In some embodiments, the nucleic acid sequence encoding the polysaccharide degrading enzyme is selected from the group consisting of SEQ ID NOs: 1-13, and homologs thereof.

In some embodiments, a LA-utilizing bacterium is genetically-engineered to produce a single polysaccharide-degrading enzyme. In other embodiments, a LA-utilizing bacterium is genetically-engineered to produce a plurality of different polysaccharide-degrading enzymes. A “plurality” indicates at least two. In some embodiments, in order to control the expression level of each polysaccharide-degrading enzyme when a plurality of enzymes is engineered into the bacterium, each enzyme is engineered with a Ribosome Binding Site (RBS) of different potency, as known in the art.

In some embodiments, each of the polysaccharide-degrading enzymes may be engineered with a similar or different promoter. In some embodiments, each of the polysaccharide-degrading enzymes may be engineered with a similar or different signal peptide, if such is used.

In some embodiments, a population of LA-utilizing bacteria comprising a plurality of sub-populations is generated, each sub-population is genetically-engineered to express and secrete a different polysaccharide-degrading enzyme. A “plurality” of sub-populations indicates at least two sub-populations, for example two, three, four sub-populations. The sub-populations are for use as a co-culture in processing organic waste. In some embodiments, a population comprises substantially equal amounts of each sub-population. In other embodiments, a population comprises sub-populations at various ratios (i.e. each sub-population may constitute a different percentage within the population).

As noted above, the polysaccharide-degrading enzymes engineered into the LA-utilizing bacteria may be selected from glycoside hydrolases, polysaccharide lyases and carbohydrate esterases. The broad group of polysaccharide-degrading enzymes is divided into enzyme classes and further into enzyme families according to a standard classification system (Cantarel et al. 2009 Nucleic Acids Res 37: D233-238). An informative and updated classification of such enzymes is available on the Carbohydrate-Active Enzymes (CAZy) server (www.cazy.org).

In some embodiments, the polysaccharide-degrading enzymes are enzymes that degrade polysaccharides selected from starch and non-starch plant polysaccharides.

In some embodiments, the polysaccharide-degrading enzymes engineered into the LA-utilizing bacteria are glycoside hydrolases.

In some embodiments, the glycoside hydrolases comprise a cellulase. The cellulase may be an exocellulase or an endocellulase. In some embodiments, a LA-utilizing bacterium of the present invention is genetically modified to express and secrete a single cellulase. In other embodiments, the LA-utilizing bacterium is genetically modified to express and secrete a plurality of different cellulases. In some embodiments, a cellulase may be selected from, but not limited to: endo-(1,4)-β-D-glucanase, exo-(1,4)-β-D-glucanase, β-glucosidases, Carboxymethylcellulase (CMCase); endoglucanase; cellobiohydrolase; avicelase, celludextrinase, cellulase A, cellulosin AP, alkali cellulase, and pancellase SS. Each possibility is a separate embodiment.

In some embodiments, a nucleic acid encoding for a cellulase may have a nucleic acid sequence as denoted by any one of SEQ ID Nos: 12-13, or homologs thereof. Each possibility is a separate embodiment. In some embodiments, a cellulase may have an amino acid sequence as denoted by nay one of SEQ ID NOs: 25-26, or homologs thereof. Each possibility is a separate embodiment. In some embodiments, a cellulase comprises or consists of an amino acid sequence as denoted by any one of SEQ ID NOs: 25-26, or homologs thereof. Each possibility is a separate embodiment.

In additional embodiments, the glycoside hydrolases comprise a hemicellulase. In some embodiments, a LA-utilizing bacterium of the present invention is genetically modified to express and secrete a single hemicellulase. In other embodiments, the LA-utilizing bacterium is genetically modified to express and secrete a plurality of different hemicellulases. In some embodiments, the hemicellulase is a xylanase. Non-limiting examples of additional hemicellulases include arabinofuranosidases, acetyl esterases, mannanases, α-D-glucuronidases, β-xylosidases, β-mannosidases, β-glucosidases, acetyl-mannanesterases, α-galactosidases, -α-Larabinanases, and β-galactosidases. Each possibility represents a separate embodiment of the present invention.

In some embodiments, a nucleic acid encoding for a xylanase may have a nucleic acid sequence as denoted SEQ ID No: 11, or homologs thereof. In some embodiments, a xylanase may have an amino acid sequence as denoted by SEQ ID No. 24, or homologs thereof. Each possibility is a separate embodiment.

In yet additional embodiments, the glycoside hydrolases comprise an amylase. In some embodiments, a LA-utilizing bacterium of the present invention is genetically modified to express and secrete a single amylase. In other embodiments, the LA-utilizing bacterium is genetically modified to express and secrete a plurality of different amylases. In some embodiments, an amylase may be selected from, but not limited to: α-amylase; (1,4-α-D-glucan glucanohydrolase; glycogenase) β-Amylase; (1,4-α-D-glucan maltohydrolase; glycogenase; saccharogen amylase) γ-Amylase; (Glucan 1,4-α-glucosidase; amyloglucosidase; Exo-1,4-α-glucosidase; glucoamylase; lysosomal α-glucosidase and 1,4-α-D-glucan glucohydrolase. Each possibility is a separate embodiment.

In some embodiments, a nucleic acid encoding for an amylase may have a nucleic acid sequence as denoted by any one of SEQ ID Nos: 1-10, or homologs thereof. Each possibility is a separate embodiment. In some embodiments, an amylase may have an amino acid sequence as denoted by SEQ ID NOs: 14-23, or homologs thereof. Each possibility is a separate embodiment. In some embodiments, an amylase comprises or consists of an amino acid sequence as denoted by SEQ ID NOs: 14-23, or homologs thereof. Each possibility is a separate embodiment.

The polysaccharide-degrading enzymes engineered into LA-utilizing bacteria according to the present invention may be from a bacterial source. In some embodiments, the bacterial source is a thermophilic bacterium. The term “thermophilic bacterium” as used herein indicates a bacterium that thrives at temperatures higher than about 45° C., preferably above 50° C. Typically, thermophilic bacteria according to the present invention have optimum growth temperature of between about 45° C. to about 75° C., preferably about 50-70° C. Non-limiting examples of thermophilic bacterial sources for polysaccharide-degrading enzymes include: Cellulases and hemicellulases—Clostridium sp. (e.g. Clostridium thermocellum), Paenibacillus sp., Thermobifida fusca; Amylases—Bacillus sp. (e.g. Bacillus stearothermophilus), Geobacillus sp. (e.g. Geobacillus thermoleovorans), Chromohalobacter sp., Rhodothermus marinus. Each possibility is a separate embodiment.

In additional embodiments, the bacterial source of the polysaccharide-degrading enzymes is a mesophilic bacterium. The term “mesophilic bacterium” as used herein indicates a bacterium that thrives at temperatures between about 20° C. and 45° C. Non-limiting examples of mesophilic bacterial sources for polysaccharide-degrading enzymes include: Cellulases and hemicellulases—Klebsiella sp. (e.g. Klebsiella pneumonia), Cohnella sp., Streptomyces sp, Acetivibrio cellulolyticus, Ruminococcus albus; Amylases—Bacillus sp. (e.g. Bacillus amyloliquefaciens, Bacillus subtilis, Bacillus licheniformis), Lactobacillus fermentum. A person of skill in the art understands that some mesophilic bacteria (e.g. several Bacillus sp.) produce thermostable enzymes.

The polysaccharide-degrading enzymes engineered into LA-utilizing bacteria according to the present invention may also be from a fungal source. Non-limiting examples of fungal sources for polysaccharide-degrading enzymes include: Cellulases and hemicellulases—Trichoderma reesei, Humicola insolens, Fusarium oxysporum; Amylases—Aspergillus oryzae, Penicillium fellutanum, Thermomyces lanuginosu.

Additional sources for polysaccharide-degrading enzymes for use in accordance with the present invention can be found, for example, at the CAZy server mentioned above.

In some embodiments, a LA-utilizing bacterium of the present invention is genetically-engineered to produce polysaccharide-degrading enzymes having suitable activity at the same temperature and/or pH range that are suitable for growth of the LA-utilizing bacteria. In some embodiments, a suitable activity is optimal activity. In some embodiments, suitable growth is optimal growth. In some embodiments, a suitable condition is sub-optimal.

In some embodiments, a LA-utilizing bacterium of the present invention is genetically-engineered to produce polysaccharide-degrading enzymes having optimal activity at the same temperature and/or pH range that are optimal for growth of the LA-utilizing bacteria.

In some embodiments, a LA-utilizing bacterium of the present invention is genetically-engineered to produce polysaccharide-degrading enzymes having suitable activity at a temperature and/or pH range that is different from the suitable temperature and/or pH range for growth of the LA-utilizing bacteria.

In some embodiments, a LA-utilizing bacterium of the present invention is genetically-engineered to produce polysaccharide-degrading enzymes having optimal activity at a temperature and/or pH range that is different from the optimal temperature and/or pH range for growth of the LA-utilizing bacteria. For example, polysaccharide-degrading enzymes from a thermophilic source may be engineered into a LA-utilizing bacterium, which typically is a mesophile.

### Processing of Organic Waste

In some embodiments, organic wastes to be processed typically comprise endogenous D,L-lactic acid.

In some embodiments, in order to utilize the organic waste as a substrate for discrete lactic acid enantiomer(s) production, it is required to selectively remove at least the unwanted enantiomer prior to lactic acid fermentation (in order to polymerize lactic acid into polylactic acid suitable for industrial applications it should be at least about 95% optically pure, preferably at least about 99% optically pure). Removal of at least the unwanted enantiomer from the organic waste should be performed with minimal impact on the feedstock total sugar content.

In some embodiments, organic wastes to be processed also comprise complex polysaccharides and reducing sugars at varying ratios. The composition depends on the source of the waste, where some organic wastes may be more starch-rich (e.g., food waste from bakeries, mixed food waste of municipalities) and others may be rich with lignocellulosic material (e.g. agricultural waste). In some embodiments, the organic waste to be processed includes a combination of wastes from different sources.

In some embodiments, the composition of the organic waste in terms of reducing sugars and polysaccharides may be determined prior to processing using methods known in the art, including for example enzymatic assays (colorimetric, fluorometric) with glucose oxidase, hexokinase or phosphoglucose isomerase for fructose determination. Alternatively, HPLC and/or reducing sugars continuous sensors can be utilized. Total sugar analysis can be performed, for example, by phenol-sulfuric assay. The composition of the organic waste, for example percentage of at least one of starch, cellulose and hemicelluloses, may be used for selecting the LA-utilizing bacterium or the population of LA-utilizing bacteria to be contacted with the organic waste. For example, for organic wastes comprising a higher percentage of starch compared to cellulose, a cell consortium may be selected which comprises a first sub-population producing an amylase and a second sub-population producing a cellulase, where the ratio between the sub-populations is tailored to the ratio between starch and cellulose in the organic waste. A single bacterium producing all the necessary enzymes may also be used, and the enzyme dose and ratios can be altered and tailored using Ribosome Binding Sites (RBS) of different potencies, as noted above.

In some embodiments, processing of the organic waste according to the present invention typically begins with pretreatment of the organic waste to decrease particle size and increase surface area, and also to inactivate endogenous bacteria within the waste. The pretreatment may include, for example, shredding and sterilization by methods known in the art. Pretreatment may also include mincing with an equal amount of water using a waste mincer, such as, e.g., an extruder, sonicator, shredder or blender.

According to some embodiments, following pretreatment, the organic waste is mixed in a reactor (fermenter) with the LA-utilizing bacteria of the present invention and the bacterial culture is propagated under conditions suitable for lactic acid consumption by the LA-utilizing bacteria and for producing the engineered polysaccharide-degrading enzymes. In some embodiments, such conditions may include a temperature in the range of about 30-40° C. and any subranges thereof. Each possibility is a separate embodiment. In some embodiments, such conditions may include a pH in the range of about 5.5-6.5 and any subranges thereof. Each possibility is a separate embodiment.

In some embodiments, when the polysaccharide-degrading enzymes are secreted and have suitable (such as, optimum) temperature and/or pH similar to those which are suitable (such as optimal) for growth of the LA-utilizing bacteria, the result is concomitant lactic acid elimination and organic waste saccharification.

In some embodiments, when the polysaccharide-degrading enzymes are secreted and have suitable (such as, optimal) temperature and pH different from those which are suitable (such as optimal) for growth of the LA-utilizing bacteria, the enzymes are secreted but remain inactive, or partially active, until the conditions are adjusted to those suitable for enzyme activity, resulting in separate lactic acid elimination and organic waste saccharification.

In some embodiments, when the polysaccharide-degrading enzymes are non-secreted and released only after lysis of the LA-utilizing bacterial cells, the result is also separate lactic acid elimination and organic waste saccharification.

In some embodiments, the culture is maintained for sufficient time to eliminate D-lactic acid, L-lactic acid or both from the waste (depending on the type of LA-utilizing bacteria), and optionally to obtain desired level of reducing sugars (in concomitant lactic acid elimination and organic waste saccharification).

In some embodiments, the time period may range from about 2 hours to about 15 hours or any amount therebetween, preferably between 2-12 hours, or 2-10 hours, such as 2 hours, 3 hours, 4 hours, 5 hours, 6 hours, 7 hours, 8 hours, 9 hours and 10 hours.

As used herein, the term “about”, when referring to a measurable value, is meant to encompass variations of +/−10%, preferably +/−5%, more preferably, +/−1%, and still more preferably +/−0.1% from the specified value.

Organic wastes typically include nitrogen sources and other nutrients needed for the LA-producing bacteria, but such nutrients may also be supplied separately if needed.

In some embodiments, following the above processing, the amount of reducing sugars is determined. Such determination may be useful for downstream fermentation processes utilizing the reducing sugars, enabling control of the concentration of fed sugars.

### Production of Discrete Lactic Acid Enantiomer(s)

In some embodiments, the reducing sugars of the organic waste (those originally found in the waste and those released by the action of the polysaccharide-degrading enzymes) may be fermented to lactic acid by LA-producing microorganisms. To generate only one discrete enantiomer of lactic acid, the LA-producing microorganisms that are used produce only one of D-lactic acid and L-lactic acid enantiomers. The LA-producing microorganisms may produce only one enantiomer naturally, or may be genetically modified to produce only one enantiomer, for example by knocking out one or more enzymes involved in the synthesis of the undesired enantiomer.

In some embodiments, prior to lactic acid fermentation, the LA-utilizing bacteria are inactivated to avoid consumption of lactic acid produced during fermentation. Inactivation may be performed, for example, by increasing the temperature or changing the pH to a temperature/pH at which the LA-utilizing bacteria are irreversibly inactivated. Alternatively or additionally, inactivation of the LA-utilizing bacteria may be performed by cell lysis using, for example, sonication. The latter may be used when the LA-utilizing bacteria are engineered to produce non-secreted polysaccharide-degrading enzymes, and the cell lysis inactivates the bacteria concomitant with release of the enzymes. In some embodiments, inactivation encompasses sterilization or pasteurization.

As used herein, “inactivated”, indicates dead or dying cells. Typically, at least 80% of the cells are inactivated, for example at least 85% of the cells are inactivated, at least 90% of the cells are inactivated, at least 95% of the cells are inactivated, or 100% of the cells are inactivated. Each possibility represents a separate embodiment of the invention.

In some embodiments, lactic acid fermentation is carried out in the same reactor (fermenter) where processing of the organic waste by the LA-utilizing bacteria and polysaccharide-degrading enzymes was carried out. In other embodiments, fermentation is carried out in a separate reactor. In some embodiments, the processed organic waste is filtered to remove solid materials and the filtered broth is mixed with LA-producing microorganisms for the fermentation stage.

LA-producing microorganisms include various bacteria (including for example Lactobacillus species and Bacillus species) and fungi.

Typically, the fermenting step is carried out under anaerobic or microaerophilic conditions, using batch, fed-batch, continuous or semi-continuous fermentation. Each possibility represents a separate embodiment of the present invention.

In batch fermentation, the carbon substrates and other components are loaded into the reactor, and, when the fermentation is completed, the product is collected. Except for neutralizing agents for pH control, other ingredients are not added to the reaction before it is completed. The inoculum size is typically about 5-10% of the liquid volume in the reactor. The fermentation is kept at substantially constant temperature and pH, where the pH is maintained by adding a suitable neutralizing agent, such as an alkali, a carbonate or ammonia.

In fed-batch fermentation, the substrate is fed continuously or sequentially to the reactor without the removal of fermentation broth (i.e., the product(s) remain in the reactor until the end of the run). Common feeding methods include intermittent, constant, pulse-feeding and exponential feeding.

In continuous fermentation, the substrate is added to the reactor continuously at a fixed rate, and the fermentation products are taken out continuously.

In semi-continuous processes, a portion of the culture is withdrawn at intervals and fresh medium is added to the system. Repeated fed-batch culture, which can be maintained indefinitely, is another name of the semi-continuous process.

Lactic acid fermentation is typically carried out for about 1-4 days or any amount therebetween, for example, 1-2 days, or 2-4 days, or 3-4 days.

According to some embodiments, the polysaccharide-degrading enzymes may have optimal temperature and/or pH that different from those optimal for growth of the LA-utilizing bacteria that secrete them, and accordingly, the enzymes may be secreted during processing with the LA-utilizing bacteria, but they remain inactive (or partially active) and substantially do not degrade polysaccharides in the organic waste, or the degradation rate is low. In some embodiments, the optimum temperature and/or pH of the polysaccharide-degrading enzymes are similar to those that are optimal for lactic acid fermentation by the LA-producing microorganisms. According to these embodiments, the enzymes may remain inactive in the broth until the LA-producing microorganisms are added and the conditions are adjusted to allow their activation. This may result in simultaneous saccharification and fermentation.

In some embodiments, the polysaccharide-degrading enzymes may be non-secreted and released into the medium only after lysis of the LA-utilizing bacterial cells. In some embodiments, the LA-producing microorganisms are added into the medium after lysis of the LA-utilizing bacterial cells, such that simultaneous saccharification and fermentation occurs.

According to the above embodiments, the method for producing discrete lactic acid enantiomer comprises: (i) processing the organic waste to eliminate D-lactic acid, L-lactic acid or a combination thereof present in the waste by contacting the organic waste with the LA-utilizing bacterium of the present invention, or population of LA-utilizing bacteria of the present invention, under conditions suitable for lactic acid consumption by the LA-utilizing bacteria and for production of the polysaccharide-degrading enzymes; (ii) inactivating the LA-utilizing bacteria; (iii) degrading polysaccharides in the waste to release reducing sugars concomitant with fermenting the released sugars to discrete lactic acid enantiomer, by contacting the organic waste obtained in (i) with a lactic acid-producing microorganism that produces only one of D-lactic acid enantiomer and L-lactic acid enantiomer, under conditions suitable for activity of the polysaccharide-degrading enzymes produced in (i) and for lactic acid fermentation by the LA-producing microorganism; and (iv) recovering the discrete lactic acid enantiomer from the fermentation broth.

In other embodiments, the organic waste is saccharified prior to lactic acid fermentation (separate hydrolysis and fermentation).

According to these embodiments, the method for producing discrete lactic acid enantiomer comprises: (i) processing the organic waste to eliminate D-lactic acid, L-lactic acid or a combination thereof present in the waste and degrade polysaccharides in the waste to release soluble reducing sugars, by contacting the organic waste with the LA-utilizing bacterium of the present invention, or population of LA-utilizing bacteria of the present invention, under conditions suitable for lactic acid consumption by the LA-utilizing bacteria and for secretion and activity of the polysaccharide-degrading enzymes; (ii) inactivating the LA-utilizing bacteria; (iii) fermenting the soluble reducing sugars obtained in (i) with a lactic acid-producing microorganism that produces only one of D-lactic acid and L-lactic acid, to obtain discrete lactic acid enantiomer; and (iv) recovering the discrete lactic acid enantiomer from the fermentation broth.

After fermentation is completed, the broth containing lactic acid may be typically clarified by centrifugation or passed through a filter press to separate solid residue from the fermented liquid. The filtrate may be concentrated, e.g. using a rotary vacuum evaporator.

Separation and purification of lactic acid from the broth may be carried out by methods known in the art, including distillation, extraction, electrodialysis, adsorption, ion-exchange, crystallization and combinations of these methods. Several methods are reviewed, for example, in Ghaffar et al. (2014), supra; and López-Garzón et al. (2014) Biotechnol Adv., 32(5):873-904). Alternatively, recovery and conversion of lactic acid to lactide in a single step may be used (Dusselier et al. (2015) Science, 349(6243):78-80).

In some embodiments, the systems and methods disclosed herein for processing waste are particularly suitable for use with mixed food waste of commercial, industrial and municipal origin. The use of mixed food waste as substrate is particularly suitable for large-scale industrial fermentation as it is heterogeneous and hence it would contain most of the required minerals and vitamins for fermentation with bacteria of ruminal origin. Further, the systems and methods disclosed herein are advantageous over currently used methods as they exhibit low fossil fuel usage, do not use valuable arable land to grow crops for feedstock, water usage is low, as is GHG emission and further, the products obtained are biodegradable.

According to some embodiments, the systems and methods disclosed herein are further advantageous as they allow combining unit operations for an industrial process, as no externally added polysaccharide-degrading enzymes are needed to utilize waste. Rather, production of such enzymes is performed in the same vessel in which fermentation occurs.

### Production of Polylactic Acid (PLA)

In order to generate PLA that is suitable for industrial applications, the polymerization process should utilize only one enantiomer. Presence of impurities or a racemic mixture of D- and L-lactic acid results in a polymer having undesired characteristics such as low crystallinity and low melting temperature. Thus, lactic acid bacteria that produce only L-lactate enantiomer or only D-lactate enantiomer are required.

Polymerization of PLA may be carried out by methods known in the art. Known methods include polymerization via lactide (di-lactic acid) formation, and direct condensation of lactic acid monomers. Several methods are reviewed, for example, in Södergård and Stolt (2010) Industrial Production of High Molecular Weight Poly(Lactic Acid), in: Poly(Lactic Acid): Synthesis, Structures, Properties, Processing, and Applications (eds R. Auras, L.-T. Lim, S. E. M. Selke and H. Tsuji), John Wiley & Sons, Inc., Hoboken, N.J., USA. In some embodiments, the PLA is Poly-L Lactic Acid (PLLA). In some embodiments, the PLA is Poly-D Lactic Acid (PDLA).

According to some embodiments, the systems and methods disclosed herein can result in an end product, namely, poly-lactic acid (PLA) that is completely recycled (i.e., 100% recycled) from waste.

According to some embodiments, the LA-utilizing bacteria disclosed herein may be successfully utilized in various methods and systems for production of lactic acid and PLA, which can be implemented by various commercial settings, including, external outdoors, various fermenters and reactors, recycling factories, and the like.

In some embodiments, various steps in the methods may be performed in one discrete location. In some embodiments, various steps in the methods may be performed in one or more operational units, such as, fermenters and reactors. In some embodiments, various steps in the methods may be performed simultaneously or consecutively. For example, size reduction of the organic waste may be combined with sterilization step, temporally and/or spatially.

In some embodiments, steps in methods of obtaining polylactic acid from organic waste using the LA-utilizing bacteria of the present invention may include one or more of the steps of:
1. Obtaining organic waste. The organic waste may be selected from residential, commercial or industrial food waste. The organic waste may be source-separated or machine separated.
2. Size reduction of the organic waste—mincing and grinding of the organic waste can be applied to achieve a homogenous semi-solid feedstock. Further. sonication can be also be used to thereby combine size reduction with sterilization. This can be performed in the same operating unit.
3. Sterilization—can be achieved, for example, using sonication, heat, UV and/or pressure. This can be performed in-situ within a fermenter operating unit, or externally. In some embodiments, as mentioned above, this step can be combined with the step of size reduction.
4. Seeding of genetically engineered LA-utilizing bacteria—Genetically engineered LA-utilizing bacteria can be inoculated in a seed fermenter operating unit to produce enzymes. Additionally, lactate by-products from downstream processing can be added as feedstock at this step (as further detailed below). The LA-utilizing bacteria can also be cultivated on defined media, such as YEL.
5. Inoculation and co-generation of Polysaccharide degrading enzymes and CO₂₋ and consumption of racemic lactate—The seed LA-utilizing bacteria is inoculated in a production fermenter to further produce enzymes and consume racemic lactate in the organic waste.
6. Inactivation of LA-utilizing bacteria—temperature in the fermenter may be increased (for example, to 45-55° C.), to result in inactivation (sterilization) of the LA-utilizing bacteria. Advantageously, such elevated temperatures may further enhance the activity of secreted polysaccharide degrading enzymes, which have been secreted by the LA-utilizing bacteria providing improved conditions (such as optimal conditions) for their activity.
7. Inoculation with lactic acid producing organism (pure L or pure D producer)—consequently, fermentation media is depleted from racemic lactate and reducing sugars (formed by the secreted degrading enzymes) become available for the lactic acid fermentation process.
8. Recovery and purification of lactic acid—Various purification methods (or combinations thereof) can be utilized to purify the enantiomerically pure lactic acid. Such methods include, for example, micro/nano filtration, centrifugation, ion chromatography, precipitation, crystallization, and the like.
9. Polymerization to PLLA or PDLA—polymerization of the purified lactic acid to PLA is performed using methods known in the art, including polymerization via lactide (di-lactic acid) formation, and direct condensation of lactic acid monomers.

In some embodiments, recycling and reuse of downstream residual lactic acid or lactate waste formed in the process of obtaining pure lactic acid (and PLA), may be advantageously utilized using the LA-utilizing bacteria of the present invention. Such downstream residual lactic acid or lactate waste formed in the process may emerge from, for example, removing solids from fermentation broths (for example, using membrane filtration or centrifugation), the remaining solids contain useful lactate that did not separate. Additionally, when synthesizing lactides, some lactic acid molecules can undergo racemization. Such residual lactic acid and/or lactate waste fermentation can advantageously be recycled in the process, for example, by being utilized as feedstock for the LA-utilizing bacteria enzyme production. When organic acids such as lactic acid are recovered from fermentation broths, normally the yield is not 100% and there is residual diluted lactic acid. Recovering the residual organic acid is costly so instead it can be recycled and used for another fermentation batch. Recycling the residue reintroduces minerals and salts also present in the residue, which might be required for a new fermentation batch. Continuous and semi-continuous fermentation lactic acid production processes require that a sample of the fermentation broth rich with bacteria and lactic acid, be used to seed incoming sterile fermentation feedstock. The problem of utilizing organic waste in a continuous or semi-continuous process for production of lactic acid, is the unwanted enantiomer which is present in the incoming raw material. If the feedstock seeded with the rich fermentation broth the production of lactic acid starts immediately, the unwanted enantiomer remains in the process as an impurity. Thus, the use of the lactate utilizing bacteria in the lactic acid production process allows for efficient recycling of all such resources. Residual lactic acid from the recovery process and/or pasteurized/sterilized lactic acid-rich broths can be used to cultivate the bacteria and produce high quantities of enzymes for another production cycle. This can be achieved either in situ, or in a separate smaller seed fermenter. In some embodiments, a sample from the lactic-acid rich fermentation broth can be split into two portions, one is pasteurized and used to cultivate enzyme producing bacteria. The second portion is not pasteurized and contains high cell mass of bacteria can be used to seed feedstock which has been treated with LA-utilizing bacteria to remove enantiomers and/or saccharified with enzymes.

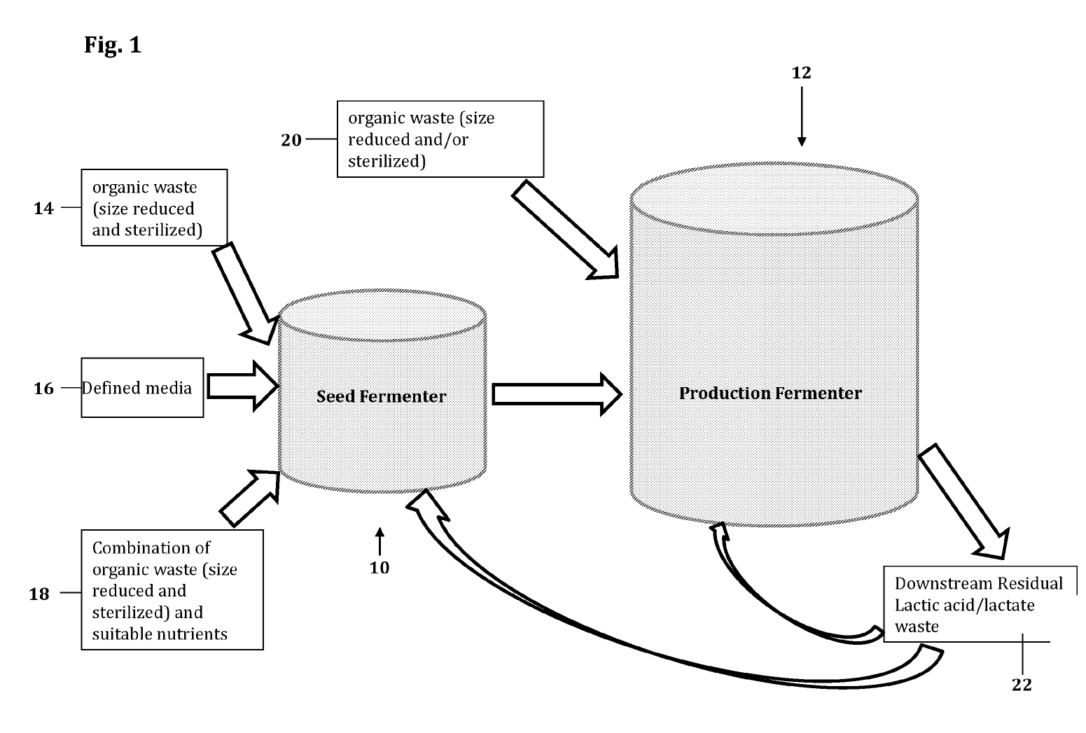

Reference is now made to FIG. 1, which is a schematic illustration of a fermentation system for producing discrete enantiomers of lactic acid and recycling of residual lactate waste, according to some embodiments. As shown in FIG. 1, the fermentation system includes a seed fermenter (10), in which genetically engineered LA-utilizing bacteria is inoculated. The cultivating medium/broth can be size reduced and sterilized/pasteurized organic waste (14) of various sources as detailed above, defined media (such as, YEL) (16) and/or a combination of size reduced and sterilized organic waste and suitable nutrients beneficial for growth of the LA-utilizing bacteria (18). In the seed chamber, co-generation of secreted polysaccharide reducing enzymes (produced by the LA-utilizing bacteria) and CO₂ is achieved, as well as lactate consumption (by the LA-utilizing bacteria). The seed LA-utilizing bacteria is then transferred to a production fermenter (12), to further produce and secrete polysaccharide reducing enzymes and consume racemic lactate. Additionally, further batches of size reduced and/or sterilized organic waste (20) may be added to the production fermenter. Next, the conditions (such as, temperature or pH) in the production fermenter are modified (for example, by elevating the temperature to 45-65° C. (for example, 55° C.)) to thereby result in inactivation of the LA-utilizing bacteria and optionally, provide enhanced working conditions to the secreted polysaccharide degrading enzymes. This results in the fermentation media to be depleted from racemic lactate and to be enriched with reducing sugars available for fermentation. LA-producing microorganisms are inoculated with the medium to produce an optically pure lactic acid enantiomer, which may then be purified and further processed, for example, by polymerization to PLA. Advantageously, downstream residual lactic acid and/or lactate waste (22) that has been formed in the process can be re-used and recycled, by being added to the seed fermenter and/or production fermenter (as indicated by the curved arrows).

According to some embodiments, there is thus provided a system and method of recycling residual lactic acid and/or lactate waste formed in the process of preparing optically pure lactic acid enantiomers.

In some embodiments, the system may include one or more sources of organic waste; one or more fermenters; a genetically modified LA-utilizing bacteria, modified to express and secrete one or more exogenous polysaccharide-degrading enzyme, wherein the fermenter is configured to allow growth of the LA-utilizing bacteria under conditions in which lactic acid is consumed by the LA-utilizing bacteria and which are suitable for expression and activity of the polysaccharide-degrading enzymes, wherein said processing eliminates D-lactic acid, L-lactic acid or both from the organic waste and degrades polysaccharides in the waste to release soluble reducing sugar; and further configured to allow conditions which inactivate the LA-utilizing bacteria, and LA-producing microorganisms that can be inoculated in the fermenter, said LA-producing microorganisms are capable of fermenting the soluble reducing sugars produced by the secreted polysaccharide-degrading enzymes to produce only one of D-lactic acid and L-lactic acid, to obtain discrete enantiomers of lactic acid; wherein downstream residual lactic acid and/or lactate waste formed in the process are added back to the one or more fermenters, to be re-used as substrate in the process.

The terms “comprises”, “comprising”, “includes”, “including”, “having” and their conjugates mean “including but not limited to”. The terms “comprises” and “comprising” are limited in some embodiments to “consists” and “consisting”, respectively. The term “consisting of” means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

The foregoing description of the specific embodiments will so fully reveal the general nature of the invention that others can, by applying current knowledge, readily modify and/or adapt for various applications such specific embodiments without undue experimentation and without departing from the generic concept, and therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed chemical structures and functions may take a variety of alternative forms without departing from the invention.

The following examples are presented in order to more fully illustrate some embodiments of the invention. They should, in no way be construed, however, as limiting the broad scope of the invention.

### EXAMPLES

### Example 1—Constructions of Suitable Vectors for Genetically Modifying Bacteria

Construction of E. coli-P. freudenreichii Shuttle Vector:
The Vectors Constructed can be Used in Both E. coli and P. freudenreichii.

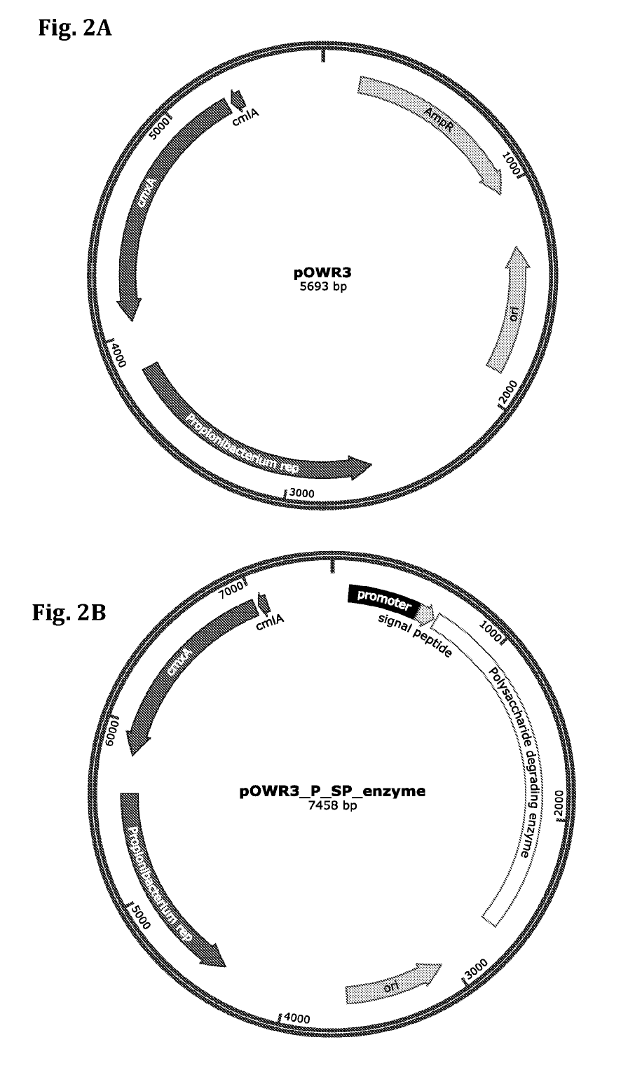

The pOWR3 vector (a schematic illustration is shown in FIG. 2A) was prepared as follows: Propionibacterium replication protein sequence (SEQ ID NO. 84) and chloramphenicol resistance genes (cmx(A) (SEQ ID NO: 85) and cml(A) (SEQ ID NO: 86) from Corynebacterium striatum pT10 plasmid) were synthesized in tandem into a vector comprising E. coli origin of replication (ori) and ampicillin resistance cassette (AmpR). This results in a ˜5.6 kbp plasmid able to replicate both in E. coli and P. freudenreichii. This vector further includes a PB origin of replication sequence. The resulting pOWR3 vector is smaller than other known P. freudenreichii expression vectors (5.6 kbp versus 6.2-8 kbp), which makes it easier to transform to bacterial cells (for example, by electroporation) and easier to use for cloning by the restriction free method. Moreover, the AmpR gene sequence region of pOWR3 is used as a cloning site, for inserting an expression cassette (as detailed below). This region is bordered (upstream and downstream) by termination signals which prevents polar effect interruptions and stabilizes the mRNA transcript (of the polysaccharide degrading enzyme) produced in the bacterial cell.

Transformation of pOWR3 Vector to P. freudenreichii.

The pOWR3 vector was transformed into P. freudenreichii by electroporation. A Turbid culture of P. freudenreichii was diluted 1:125 to fresh YEL media (gr/l: 10 yeast extract, 10 peptone, 10 lactate) for overnight culture in 30° C. under aerobic conditions. The culture (O.D 600=0.2-1.9) was placed on ice for 30 minutes, then washed twice in the same volume with ice cold water (3000 rpm 10 min). A 3^{rd} wash was with same volume of cold 10% glycerol (3000 rpm 10 min). The pellet was concentrated (0.01% of the original culture) in 10% cold glycerol. 50 μl of electro-competent P. freudenreichii were mixed in a tube with 1000 ng of the pOWR3 vector. The bacteria and vector mix were transferred to a 0.1 cm gap cuvette (Biorad) and electroporated using the following conditions: field strength—20000 V/cm, resistance—500 ohm, capacitor—25 uF. 900μ1 of YEL media was added and the bacteria were incubated for 3 hours in 30° C. under aerobic conditions, followed by spreading on YEL media+10 ugr/ml Chloramphenicol agar plates.

To construct the suitable expression vectors, allowing expression and secretion of the polysaccharide degrading enzymes, the pOWR3 vector is modified by replacing the ampicillin resistance gene with an “expression cassette” which includes, i) a promoter sequence, operably linked to (ii) nucleic acid sequence encoding for a signal peptide (SP) which is translationally linked to (iii) a nucleotide sequence encoding for a polysaccharide degrading enzyme. (FIG. 2B). When said expression cassette is expressed in the target cell, under the control of the promoter sequence, the signal peptide is translationally linked in-frame to the polysaccharide degrading enzyme. In other words, a chimeric protein which includes the SP peptide sequence and the polysaccharide degrading enzyme peptide sequence is expressed in the cell.

By utilizing such expression cassette, a matrix of suitable combinations of promoter-SP-polysaccharide degrading enzyme can be utilized.

### Example 2—Construction of Suitable Expression Cassettes and Expression Vectors for Genetically Modifying Bacteria to Express and Secrete Polysaccharide Degrading Enzyme

Listed below are exemplary polysaccharide degrading enzymes (Glucoamylases, cellulases and hemicellulases (Xylanases), signal peptides and promoters used for creating expression cassettes used in expression vectors.

The following exemplary enzymes listed below in Table 1, are used in expression cassettes and expression vectors for genetically modifying bacteria. The enzymes sequences are obtained from different sources (organisms). The original signal peptide of each of the listed enzymes is removed and is replaced by the foreign (different) signal peptide.

TABLE 1
Exemplary polysaccharide degrading enzymes

| | | | | | | Optimal |
|---|---|---|---|---|---|---|
| SEQ ID | SEQ ID | | | GI/accession | Optimal | Temp. |
| NO (nt) | NO. (AA) | Organism name | origin | number | pH | (° C.) |
| 1 | 14 | Saccharomycopsis fibuligera | eukaryote | 113795 | 5-6 | 45 |
| 2 | 15 | Aspergillus niger | eukaryote | 30025851 | 4-5 | 55 |
| 3 | 16 | Clostridium SP.5 G000 | Gram+ | 231542 | 5.5 | 60 |
| 4 | 17 | Clostiridium thermohydrosulfuricum | Gram+ | 490533596 | 5 | 70 |
| 5 | 18 | Clostridium thermoamylolyticum | Gram+ | 34146785 | 5 | 65 |
| 6 | 19 | Thermoanaerobacter tengcongensis MB4 | Gram+ | 20516826 | 5 | 75 |
| 7 | 20 | Picrophilus torridus DSM 9790 | archaea | 48431212 | 5 | 75 |
| 8 | 21 | Picrophilus torridus DSM 9790 | archaea | 48430085 | 5 | 50 |
| 9 | 22 | Caulobacter crescentus CB15 | Gram− | AB813000 | 5 | 40 |
| 10 | 23 | Bacillus licheniformis | Gram+ | CAA01355 | 7 | 37 |
| 11 | 24 | Thermobifida Fusca TM51 (Xylanase) | Gram+ | EOR70069 | 5-6.5 | 50-60 |
| 12 | 25 | Beta-1,4-Endoglucanase | Gram+ | P26222 | 5-6.5 | 50-60 |
| | | [Thermomonospora Fusca] | | | | |
| | | (Cellulase) | | | | |
| 13 | 26 | Endoglucanase | Gram+ | WP_015924277.1 | 7 | 50 |
| | | [Clostridium Cellulolyticum] | | | | |
| | | (Cellulase) | | | | |

The following promoter regions are utilized in the construction of various expression cassettes and expression vectors:
PFREUD_04850 promoter from the Propionibacterium frudenreichii ATCC 9614 (SEQ ID NO: 27)
PFRUD_04850 promoter from the Propionibacterium frudenreichii BIA118 (SEQ ID NO: 28)
PFREUD_22150 promoter from the Propionibacterium frudenreichii ATCC 9614 (SEQ ID NO: 29)
PFREUD_08450, promoter from Propionibacterium frudenreichii ATCC 9614. (SEQ ID NO: 30)
PFREUD_18290 from Propionibacterium frudenreichii ATCC 9614. (SEQ ID NO: 31).
pGRO promoter (pgro plasmid from Propionibacterium acidipropionici) (SEQ ID NO: 32).

The following exemplary signal peptides (SP) amino acid sequences are utilized in the construction of various of expression cassettes:

>CBL57338 surface layer protein A (S-layer protein A) PFREUD_18290 [Propionibacterium freudenreichii subsp. shermanii CIRM-BIA1] (SED ID NO: 87 (amino acid sequence), SEQ ID NO: 180 (nucleotide sequence)).

>CBL56016.1 cell-wall peptidases, NlpC/P60 family secreted protein PFREUD_04850 [Propionibacterium freudenreichii subsp. shermanii CIRM-BIA1] (SEQ ID NO: 88 (amino acid sequence), SEQ ID NO: 181 (nucleotide sequence)).

>CBL56360.1 DSBA oxidoreductase [Propionibacterium freudenreichii subsp. shermanii CIRM-BIA1] (SEQ ID NO: 89 (amino acid sequence), SEQ ID NO: 182 (nucleotide sequence)).

CBL57333.1 Hypothetical protein PFREUD_18250 [Propionibacterium freudenreichii subsp. shermanii CIRM-BIA1] (SEQ ID NO: 90 (amino acid sequence), SEQ ID NO: 183 (nucleotide sequence))

gi|296921799|emb|CBL56359.1| thiredoxine like membrane protein [Propionibacterium freudenreichii subsp. shermanii CIRM-BIA1] (SEQ ID NO:91 (amino acid sequence), SEQ ID NO: 184 (nucleotide sequence))

>gi|296923311|emb|CBL57911.1| drug exporters of the RND superfamily [Propionibacterium freudenreichii subsp. shermanii CIRM-BIA1] (SEQ ID NO:92 (amino acid sequence), SEQ ID NO: 185 (nucleotide sequence))

>gi|296921405|emb|CBL55958.1| Putative carboxylic ester hydrolase [Propionibacterium freudenreichii subsp. shermanii CIRM-BIA1 (SEQ ID NO: 93 (amino acid sequence), SEQ ID NO: 186 (nucleotide sequence)).

gi|296921680|emb|CBL56237.1| Hypothetical secreted protein [Propionibacterium freudenreichii subsp. shermanii CIRM-BIA1] (SEQ ID NO:94 (amino acid sequence), SEQ ID NO: 187 (nucleotide sequence)).

>gi|296922532|emb|CBL57105.1| S-layer domain protein domain protein precursor [Propionibacterium freudenreichii subsp. shermanii CIRM-BIA1] (SEQ ID NO: 95 (amino acid sequence), SEQ ID NO: 188 (nucleotide sequence)).

>gi|296922233|emb|CBL56805.1| ABC transporter [Propionibacterium freudenreichii sub sp. shermanii CIRM-BIA1] MRLARRVAAVLLASVLALTVASCAGAARSAPSL (SEQ ID NO:96 (amino acid sequence), SEQ ID NO: 189 (nucleotide sequence)).

>CBL57324.1 Hypothetical protein PFREUD_18170 [Propionibacterium freudenreichii subsp. shermanii CIRM-BIA1] (SEQ ID NO: 97).

>CBL57805.1 polar amino acid ABC transporter, binding protein [Propionibacterium freudenreichii subsp. shermanii CIRM-BIA1] (SEQ ID NO: 98).

>CBL57413.1 Sortase family protein [Propionibacterium freudenreichii subsp. shermanii CIRM-BIA1] (SEQ ID NO: 99).

>CBL57271.1 Probable multidrug resistance transporter, MFS superfamily [Propionibacterium freudenreichii subsp. shermanii CIRM-BIA1] (SEQ ID NO: 100).

>CBL57337.1 Hypothetical protein PFREUD_18280 [Propionibacterium freudenreichii subsp. shermanii CIRM-BIA1] (SEQ ID NO: 101).

gi|296922302|emb|CBL56874.1| Putative peptidyl-prolyl cis-trans isomerase, FKBP-type (Precursor) [Propionibacterium freudenreichii subsp. shermanii CIRM-BIA1] CIRM-BIA1 (SEQ ID NO: 102).

gi|296923259|emb|CBL57856.1| Extracellular solute-binding protein precursor [Propionibacterium freudenreichii subsp. shermanii CIRM-BIA1] (SEQ ID NO: 103).

gi|296923146|emb|CBL57733.1| Hypothetical protein PFREUD_22300 [Propionibacterium freudenreichii subsp. shermanii CIRM-BIA1] (SEQ ID NO: 104).

gi|296921378|emb|CBL55931.1| Hypothetical protein PFREUD_03980 [Propionibacterium freudenreichii subsp. shermanii CIRM-BIA1] (SEQ ID NO: 105).

gi|296921927|emb|CBL56487.1| Hypothetical membrane protein [Propionibacterium freudenreichii subsp. shermanii CIRM-BIA1] (SEQ ID NO: 106).

gi|296922947|emb|CBL57529.1| carboxypeptidase (serine-type D-Ala-D-Ala carboxypeptidase) (D-alanyl-D-alanine-carboxypeptidase) [Propionibacterium freudenreichii subsp. shermanii CIRM-BIA1] (SEQ ID NO: 107).

>gi|296923326|emb|CBL57926.1| ABC transporter, substrate-binding protein [Propionibacterium freudenreichii subsp. shermanii CIRM-BIA1] (SEQ ID NO: 108).

>gi|296923322|emb|CBL57922.1| Hypothetical protein PFREUD_24060 [Propionibacterium freudenreichii subsp. shermanii CIRM-BIA1] (SEQ ID NO: 109).

>gi|296922617|emb|CBL57194.1| Hypothetical protein PFREUD_16830 [Propionibacterium freudenreichii subsp. shermanii CIRM-BIA1] (SEQ ID NO: 110).

>gi|296922915|emb|CBL57497.1| secreted glycosyl hydrolase [Propionibacterium freudenreichii subsp. shermanii CIRM-BIA1] (SEQ ID NO: 111).

>gi|296921377|emb|CBL55930.1| Hypothetical protein PFREUD_03970 [Propionibacterium freudenreichii subsp. shermanii CIRM-BIA1] (SEQ ID NO: 112).

>gi|296922301|emb|CBL56873.1| peptidyl-prolyl cis-trans isomerase [Propionibacterium freudenreichii subsp. shermanii CIRM-BIA1] (SEQ ID NO: 113).

>gi|296922866|emb|CBL57446.1| Hypothetical protein PFREUD_19530 [Propionibacterium freudenreichii subsp. shermanii CIRM-BIA1] (SEQ ID NO: 114).

>gi|296922346|emb|CBL56918.1| Hypothetical protein PFREUD_14190 [Propionibacterium freudenreichii subsp. shermanii CIRM-BIA1] (SEQ ID NO: 115).

>gi|296921344|emb|CBL55897.1| Hypothetical protein PFREUD_03630 [Propionibacterium freudenreichii subsp. shermanii CIRM-BIA1] (SEQ ID NO: 116).

>gi|296922653|emb|CBL57230.1| Hypothetical protein PFREUD_17190 [Propionibacterium freudenreichii subsp. shermanii CIRM-BIA1] (SEQ ID NO: 117).

>gi|296921113|emb|CBL55660.1| extracellular protein without function [Propionibacterium freudenreichii subsp. shermanii CIRM-BIA1] (SEQ ID NO: 118).

>gi|296921687|emb|CBL56244.1| Hypothetical protein PFREUD_07130 [Propionibacterium freudenreichii subsp. shermanii CIRM-BIA1] (SEQ ID NO: 119).

>gi|296921593|emb|CBL56147.1| Hypothetical protein PFREUD_06170 [Propionibacterium freudenreichii subsp. shermanii CIRM-BIA1] (SEQ ID NO: 120).

>gi|296922523|emb|CBL57096.1| Hypothetical protein PFREUD_15980 [Propionibacterium freudenreichii subsp. shermanii CIRM-BIA1] (SEQ ID NO: 121).

>gi|296921018|emb|CBL55556.1| large surface protein A [Propionibacterium freudenreichii subsp. shermanii CIRM-BIA1] (SEQ ID NO: 122).

>gi|296922853|emb|CBL57433.1| Penicillin-binding protein

(Transglycosylase/transpeptidase) [Propionibacterium freudenreichii subsp. shermanii CIRM-BIA1] (SEQ ID NO: 123).

>gi|296921190|emb|CBL55739.1| Hypothetical protein PFREUD_02240 [Propionibacterium freudenreichii subsp. shermanii CIRM-BIA1] (SEQ ID NO: 124).

>gi|296922847|emb|CBL57427.1| multicopper oxidase [Propionibacterium freudenreichii subsp. shermanii CIRM-BIA1] (SEQ ID NO: 125).

>gi|296923003|emb|CBL57585.1| Regulator of chromosome condensation [Propionibacterium freudenreichii subsp. shermanii CIRM-BIA1] (SEQ ID NO: 126).

>gi|296923209|emb|CBL57803.1| polar amino acid ABC transporter, binding protein [Propionibacterium freudenreichii subsp. shermanii CIRM-BIA1] (SEQ ID NO: 127).

>gi|296923210|emb|CBL57804.1| polar amino acid ABC transporter, binding protein component [Propionibacterium freudenreichii subsp. shermanii CIRM-BIA1] (SEQ ID NO: 128).

>gi|296921884|emb|CBL56444.1| ABC-transporter metal-binding lipoprotein [Propionibacterium freudenreichii subsp. shermanii CIRM-BIA1] (SEQ ID NO: 129).

>gi|296921649|emb|CBL56206.1| iron ABC transport system, solute-binding protein precursor [Propionibacterium freudenreichii subsp. shermanii CIRM-BIA1] (SEQ ID NO: 130).

>gi|296922500|emb|CBL57073.1| Peptidase M23B family/metalloendopeptidase [Propionibacterium freudenreichii subsp. shermanii CIRM-BIA1] (SEQ ID NO: 131).

>gi|296922061|emb|CBL56625.1| transporter [Propionibacterium freudenreichii subsp. shermanii CIRM-BIA1] (SEQ ID NO: 132).

>gi|296921977|emb|CBL56539.1| Hypothetical protein PFREUD_10150 [Propionibacterium freudenreichii subsp. shermanii CIRM-BIA1] (SEQ ID NO: 133).

>gi|296921310|emb|CBL55863.1| Hypothetical protein PFREUD_03320 [Propionibacterium freudenreichii subsp. shermanii CIRM-BIA1] (SEQ ID NO: 134).

>gi|296921967|emb|CBL56527.1| ABC transporter, substrate binding protein [Propionibacterium freudenreichii subsp. shermanii CIRM-BIA1] (SEQ ID NO: 135).

>gi|296921196|emb|CBL55745.1| Hypothetical protein PFREUD_02300 [Propionibacterium freudenreichii subsp. shermanii CIRM-BIA1] (SEQ ID NO: 136).

>gi|296921666|emb|CBL56223.1| Hypothetical secreted protein [Propionibacterium freudenreichii subsp. shermanii CIRM-BIA1] (SEQ ID NO:137).

>gi|296921230|emb|CBL55780.1| ABC transporter, binding lipoprotein [Propionibacterium freudenreichii subsp. shermanii CIRM-BIA1] (SEQ ID NO: 138).

>gi|296923102|emb|CBL57689.1| polar amino acid ABC transporter, binding protein component [Propionibacterium freudenreichii subsp. shermanii CIRM-BIA1] (SEQ ID NO: 139).

>gi|296923079|emb|CBL57663.1| Sulfate-binding protein precursor [Propionibacterium freudenreichii subsp. shermanii CIRM-BIA1] (SEQ ID NO: 140).

>gi|296921569|emb|CBL56123.1| solute binding protein of the ABC transport system [Propionibacterium freudenreichii subsp. shermanii CIRM-BIA1] (SEQ ID NO: 141).

>gi|296922906|emb|CBL57488.1| Phosphate-binding transport protein of ABC transporter system [Propionibacterium freudenreichii subsp. shermanii CIRM-BIA1] (SEQ ID NO: 142).

>gi|296920974|emb|CBL55511.1| membrane protein (s-layer) [Propionibacterium freudenreichii subsp. shermanii CIRM-BIA1] (SEQ ID NO: 143).

>gi|296922551|emb|CBL57124.1| Hypothetical secreted protein [Propionibacterium freudenreichii subsp. shermanii CIRM-BIA1] (SEQ ID NO: 144).

>gi|296923279|emb|CBL57879.1| penicillin-binding protein (peptidoglycan glycosyltransferase) [Propionibacterium freudenreichii subsp. shermanii CIRM-BIA1] (SEQ ID NO: 145).

>gi|296921112|emb|CBL55659.1| ATP-binding region, ATPase-like:Histidine kinase, Histidine kinase A-like precursor [Propionibacterium freudenreichii subsp. shermanii CIRM-BIA1] (SEQ ID NO: 146)

>gi|296922314|emb|CBL56886.1| Cobalt permease [Propionibacterium freudenreichii subsp. shermanii CIRM-BIA1] (SEQ ID NO: 147).

>gi|296922614|emb|CBL57191.1| Hypothetical protein PFREUD_16800 [Propionibacterium freudenreichii subsp. shermanii CIRM-BIA1] (SEQ ID NO: 148).

>gi|296921959|emb|CBL56519.1| Secreted protease with a PDZ domain [Propionibacterium freudenreichii subsp. shermanii CIRM-BIA1] (SEQ ID NO: 149).

>gi|296921835|emb|CBL56395.1| membrane protein [Propionibacterium freudenreichii subsp. shermanii CIRM-BIA1] (SEQ ID NO: 150).

>gi|296921079|emb|CBL55620.1| secreted transglycosydase [Propionibacterium freudenreichii subsp. shermanii CIRM-BIA1] (SEQ ID NO: 151).

>gi|296922047|emb|CBL56611.1| Leucine-, isoleucine-, valine-, threonine-, and alanine-binding protein [Precursor] (LIVAT-BP) [Propionibacterium freudenreichii subsp. shermanii CIRM-BIA1] (SEQ ID NO: 152).

>gi|296922667|emb|CBL57244.1| Hypothetical protein PFREUD_17340 [Propionibacterium freudenreichii subsp. shermanii CIRM-BIA1] (SEQ ID NO: 153).

>gi|296922395|emb|CBL56967.1| Hypothetical protein PFREUD_14680 [Propionibacterium freudenreichii subsp. shermanii CIRM-BIA1] (SEQ ID NO: 154).

>gi|296921224|emb|CBL55774.1| Hypothetical secreted protein [Propionibacterium freudenreichii subsp. shermanii CIRM-BIA1] (SEQ ID NO: 155).

>gi|296921129|emb|CBL55676.1| ABC transporter permease [Propionibacterium freudenreichii subsp. shermanii CIRM-BIA1] (SEQ ID NO: 156).

>gi|296921974|emb|CBL56536.1| Exopolysaccharide biosynthesis protein precursor (related to N-acetylglucosamine-1-phosphodiester alpha-N-acetylglucosaminidase precursor) [Propionibacterium freudenreichii subsp. shermanii CIRM-BIA1] (SEQ ID NO: 157).

>gi|296921168|emb|CBL55717.1| Hypothetical protein PFREUD_02020 [Propionibacterium freudenreichii subsp. shermanii CIRM-BIA1] (SEQ ID NO: 158).

>gi|296921340|emb|CBL55893.1| Hypothetical secreted protein [Propionibacterium freudenreichii subsp. shermanii CIRM-BIA1] (SEQ ID NO: 159).

>gi|296923123|emb|CBL57710.1| Hypothetical membrane protein [Propionibacterium freudenreichii subsp. shermanii CIRM-BIA1] (SEQ ID NO: 160).

>gi|296922282|emb|CBL56854.1| Hypothetical secreted protein [Propionibacterium freudenreichii subsp. shermanii CIRM-BIA1] (SEQ ID NO: 161).

>gi|296921324|emb|CBL55877.1| Hypothetical secreted protein [Propionibacterium freudenreichii subsp. shermanii CIRM-BIA1] (SEQ ID NO: 162).

>gi|296922572|emb|CBL57145.1| ABC transporter glycine betaine [Propionibacterium freudenreichii subsp. shermanii CIRM-BIA1] (SEQ ID NO: 163).

>gi|296922949|emb|CBL57531.1| Hypothetical protein PFREUD_20380 [Propionibacterium freudenreichii subsp. shermanii CIRM-BIA1] (SEQ ID NO: 164).

>gi|296921638|emb|CBL56194.1| ABC transport system component [Propionibacterium freudenreichii subsp. shermanii CIRM-BIA1] (SEQ ID NO: 165).

>gi|296922507|emb|CBL57080.1| Hypothetical protein PFREUD_15820 [Propionibacterium freudenreichii subsp. shermanii CIRM-BIA1] (SEQ ID NO: 166).

>gi|296921133|emb|CBL55680.1| Hypothetical protein PFREUD_01650 [Propionibacterium freudenreichii subsp. shermanii CIRM-BIA1] (SEQ ID NO: 167).

>gi|296921157|emb|CBL55704.1| Hypothetical secreted protein [Propionibacterium freudenreichii subsp. shermanii CIRM-BIA1] (SEQ ID NO: 168).

>gi|296921592|emb|CBL56146.1| ABC transporter-associated permease [Propionibacterium freudenreichii subsp. shermanii CIRM-BIA1] (SEQ ID NO: 169).

>gi|296923131|emb|CBL57718.1| Hypothetical protein PFREUD_22150 [Propionibacterium freudenreichii subsp. shermanii CIRM-BIA1] (SEQ ID NO: 170).

>gi|296921360|emb|CBL55913.1| Hypothetical outer membrane protein [Propionibacterium freudenreichii subsp. shermanii CIRM-BIA1] (SEQ ID NO: 171).

>gi|296921416|emb|CBL55969.1| Hypothetical protein PFREUD_04350 [Propionibacterium freudenreichii subsp. shermanii CIRM-BIA1] (SEQ ID NO: 172).

>gi|296923120|emb|CBL57707.1| Hypothetical protein PFREUD_22040 [Propionibacterium freudenreichii subsp. shermanii CIRM-BIA1] (SEQ ID NO: 173).

>gi|296921006|emb|CBL55544.1| Hypothetical protein PFREUD_00440 [Propionibacterium freudenreichii subsp. shermanii CIRM-BIA1] (SEQ ID NO: 174).

>gi|296921415|emb|CBL55968.1| Carboxylic ester hydrolase [Propionibacterium freudenreichii subsp. shermanii CIRM-BIA1] (SEQ ID NO: 175).

>gi|296922150|emb|CBL56718.1| ABC transporter substrate-binding protein [Propionibacterium freudenreichii subsp. shermanii CIRM-BIA1] (SEQ ID NO: 176).

>gi|296921167|emb|CBL55716.1| Hypothetical secreted protein [Propionibacterium freudenreichii subsp. shermanii CIRM-BIA1] (SEQ ID NO: 177).

>gi|296923127|emb|CBL57714.1| Hypothetical protein PFREUD_22110 [Propionibacterium freudenreichii subsp. shermanii CIRM-BIA1] (SEQ ID NO: 178).

>gi|296921375|emb|CBL55928.1| Hypothetical protein PFREUD_03950 [Propionibacterium freudenreichii subsp. shermanii CIRM-BIA1] (SEQ ID NO: 179).

### Example 3—Preparation of Expression Cassettes for Expressing Various Polysaccharide Degrading Enzymes in PB, Utilizing Different Combinations of Signal Peptides and Promoters

The nucleotide sequences inserts of the various promoters and polysaccharide degrading enzymes were obtained using PCR reactions utilizing suitable primers (listed in Table 2) and a corresponding template. A Q5 High-Fidelity DNA Polymerase (New England Biolabs, M0491) was used for the PCR reaction, according to manufacture instructions. PCR conditions were as follows: initial denaturation—98° C. for 1 minute, secondary denaturation—98° C. for 30 seconds, annealing −60° C. for 30 seconds, elongation—72° C. for 50 seconds. PCR programs run for 30 cycles (secondary initiation step to elongation step). PCR products were tested on agarose gel and purified using Wizard PCR cleanup kit (Promega).

### Example 3.1—Expression of Glucoamylase Gi|13795 from Saccharomycopsis Fibuligera with a PFREUD_18290 Promoter and a PFREUD_18290 Signal Peptide

The sequence of the promoter and signal peptide were amplified from P. freudenreichii using primers 1 and 2 (Table 2, below). The glucoamylase was amplified using primers 3 and 4 (Table 2). All glucoamylases were synthesized with an addition of 21 nucleotides after the stop codon, to enable the use of a shared reverse primer for their amplification. (primer 4). The DNA sequence of the expression cassette (i.e., insert) thus obtained is as denoted by SEQ ID NO: 33.

### Example 3.2—Expression of Glucoamylase Gi30025851 from Aspergillus niger Combined with a PFREUD_18290 Promoter and a PFREUD_18290 Signal Peptide

The sequence of the promoter and signal peptide were amplified from P. freudenreichii using primers 1 and 5 (Table 2). The glucoamylase was amplified using primers 6 and 4. The DNA sequence of the expression cassette (i.e., insert) thus obtained is as denoted by SEQ ID NO: 34.

### Example 3.3—Expression of Glucoamylase Gi231542 from Clostridium SP.5 G000 Combined with a PFREUD_18290 Promoter and a PFREUD_18290 Signal Peptide

The sequence of the promoter and signal peptide were amplified from P. freudenreichii using primers 1 and 7 (Table 2). The glucoamylase was amplified using primers 8 and 4. The DNA sequence of the expression cassette (i.e., insert) thus obtained is as denoted by SEQ ID NO: 35.

### Example 3.4—Expression of Glucoamylase Gi490533596 from Clostiridium Thermohydrosulfuricum Combined with a PFREUD_18290 Promoter and a PFREUD_18290 Signal Peptide

The sequence of the promoter and signal peptide were amplified from P. freudenreichii using primers 1 and 9 (Table 2). The glucoamylase was amplified using primers 10 and 4. The DNA sequence of the expression cassette (i.e., insert) thus obtained is as denoted by SEQ ID NO: 36.

### Example 33—Expression of Glucoamylase Gi34146785 from Clostridium thermoamylolyticum Combined with a PFREUD_18290 Promoter and a PFREUD_18290 Signal Peptide

The sequence of the promoter and signal peptide were amplified from P. freudenreichii using primers 1 and 11 (Table 2). The glucoamylase was amplified using primers 12 and 4. The DNA sequence of the expression cassette (i.e., insert) thus obtained is as denoted by SEQ ID NO: 37.

### Example 3.6—Expression of Glucoamylase Gi48431212 from Picrophilus torridus DSM 9790 Combined with a PFREUD_18290 Promoter and a PFREUD_18290 Signal Peptide

The sequence of the promoter and signal peptide were amplified from P. freudenreichii using primers 1 and 13 (Table 2). The glucoamylase was amplified using primers 14 and 4. The DNA sequence of the expression cassette (i.e., insert) thus obtained is as denoted by SEQ ID NO: 38.

### Example 3.7—Expression of Glucoamylase Gi|13795 from Saccharomycopsis fibuligera Combined with a PFREUD_04850 Promoter and a PFREUD_04850 Signal Peptide

The sequence of the promoter and signal peptide were amplified from P. freudenreichii using primers 15 and 16 (Table 2). The glucoamylase was amplified using primers 3 and 4. All glucoamylases were synthesized with an addition of 21 nucleotides after the stop codon, to enable the use of a shared reverse primer for their amplification. (primer 4). The DNA sequence of the expression cassette (i.e., insert) thus obtained is as denoted by SEQ ID NO: 39.

### Example 3.8—Expression of Glucoamylase Gi30025851 from Aspergillus niger Combined with a PFREUD_04850 Promoter and a PFREUD_04850 Signal Peptide

The sequence of the promoter and signal peptide was amplified from P. freudenreichii using primers 15 and 17 (Table 2). The glucoamylase was amplified using primers 6 and 4. The DNA sequence of the expression cassette (i.e., insert) thus obtained is as denoted by SEQ ID NO: 40.

### Example 3.9—Expression of Glucoamylase Gi231542 from Clostridium SP.5 G000 Combined with a PFREUD_04850 Promoter and a PFREUD_04850 Signal Peptide

The sequences of the promoter and signal peptide were amplified from P. freudenreichii using primers 15 and 18 (Table 2). The glucoamylase was amplified using primers 8 and 4. The DNA sequence of the expression cassette (i.e., insert) thus obtained is as denoted by SEQ ID NO: 41.

### Example 3.10—Expression of Glucoamylase Gi490533596 from Clostiridium Thermohydrosulfuricum Combined with a PFREUD_04850 Promoter and a PFREUD_04850 Signal Peptide

The sequences of the promoter and signal peptide were amplified from P. freudenreichii using primers 15 and 19 (Table 2). The glucoamylase was amplified using primers 10 and 4. The DNA sequence of the expression cassette (i.e., insert) thus obtained is as denoted by SEQ ID NO: 42.

### Example 3.11—Expression of Glucoamylase Gi34146785 from Clostridium thermoamylolyticum Combined with a PFREUD_04850 Promoter and a PFREUD_04850 Signal Peptide

The sequence of the promoter and signal peptide was amplified from P. freudenreichii using primers 15 and 20 (Table 2). The glucoamylase was amplified using primers 12 and 4. The DNA sequence of the expression cassette (i.e., insert) thus obtained is as denoted by SEQ ID NO: 43.

### Example 3.12—Expression of Glucoamylase Gi48431212 from Picrophilus torridus DSM 9790 Combined with a PFREUD_04850 Promoter and a PFREUD_04850 Signal Peptide

The sequence of the promoter and signal peptide was amplified from P. freudenreichii using primers 15 and 21 (Table 2). The glucoamylase was amplified using primers 14 and 4. The DNA sequence of the expression cassette (i.e., insert) thus obtained is as denoted by SEQ ID NO: 44.

### Example 3.13—Expression of Glucoamylase Gi30025851 from Aspergillus niger Combined with a PFREUD_08450 Promoter and a PFREUD_04850 Signal Peptide

The sequence of PFREUD_08450 promoter was amplified from P. freudenreichii using primers 22 and 23 (Table 2). The amplified oligo sequence is cloned using restriction free cloning method into a pOWR3 vector already containing a PFREUD_04850 signal peptide and the glucoamylase sequence (from example 3.8). The resulting vector is inserted and amplified in DH5alfa bacteria. Then transferred to dam\dcm-E. coli strain, propagated and purified, to produce non-methylated vector. The DNA sequence of the insert is as denoted by SEQ ID NO: 45.

### Example 3.14—Expression of Glucoamylase Gi30025851 from Aspergillus niger Combined with a PFREUD_04850 Promoter and PFREUD_18250 Signal Peptide

The sequence of PFREUD_18250 signal peptide was amplified from P. freudenreichii using primers 24 and 25 (Table 2). The amplified oligo sequence is cloned using restriction free cloning method into a pOWR3 vector already containing a PFREUD_04850 promoter and the glucoamylase sequence (from example 3.8). The resulting vector is inserted and amplified in DH5alfa bacteria. Then transferred to dam\dcm-E. coli strain, propagated and purified, to produce non-methylated vector. The DNA sequence of the insert is as denoted by SEQ ID NO: 46.

### Example 3.15—Expression of Xylanase EOR70069-F from Thermobifida fusca TM51 Combined with a PFREUD_04850 Promoter and a PFREUD_04850 Signal Peptide

The sequence of the promoter and signal peptide was amplified from P. freudenreichii using primers 15 and 26 (Table 2). The Xylanase was amplified using primers 27 and 28. The DNA sequence of the expression cassette (i.e., insert) thus obtained is as denoted by SEQ ID NO: 47.

### Example 3.16—Expression of Cellulase P26222 from Thermomonospora Fusca Combined with a PFREUD_04850 Promoter and a PFREUD_04850 Signal Peptide

The sequence of the promoter and signal peptide was amplified from P. freudenreichii using primers 15 and 29 (Table 2). The glucoamylase was amplified using primers 30 and 31. The DNA sequence of the expression cassette (i.e., insert) thus obtained is as denoted by SEQ ID NO: 48.

### Example 3.17—Expression of Cellulase WP_015924277.1 from Clostridium Cellulolyticum Combined with a PFREUD_04850 Promoter and a PFREUD_04850 Signal Peptide

The sequence of the promoter and signal peptide was amplified from P. freudenreichii using primers 15 and 32 (Table 2). The glucoamylase was amplified using primers 33 and 34. The DNA sequence of the expression cassette (i.e., insert) thus obtained is as denoted by SEQ ID NO: 49.

TABLE 2

| Primers | | PCR primer | Nucleotide Sequence |
|---|---|---|---|
| 1 | P18290-F | Forward | GATCGGCACGTAAGAGGTTCCAACTTTCACCCCGAG |
| | | | GTCCACACGCCGG (SEQ ID NO: 50) |
| 2 | P18290-R1 | Reverse | GGC GCG CTT GTT CAG GGG GCC CTC CTG CAG GGC |
| | | | ATT CGC AGG CGC CAT G (SEQ ID NO: 51) |
| 3 | seq1-F | Forward | CTGCAGGAGGGCCCCCTGAACAAGCGCGCC (SEQ ID |
| | | | NO: 52) |
| 4 | Amylase-R | Reverse | CTA TTT CGT TCA TCC ATA GTT GCC TGA CTC ATG |
| | | | GGA TTC AGT TGT AGG TG (SEQ ID NO: 53) |
| 5 | p18290-R2 | Reverse | CCA CGA GTC CCA GGT GGC ACG CTT CGA GAT |
| | | | GGC ATT CGC AGG CGC CAT G (SEQ ID NO: 54) |
| 6 | seq2-F | Forward | ATCTCGAAGCGTGCCACCTGGGACTCGTGG (SEQ ID |
| | | | NO: 55) |
| 7 | p18290-R3 | Reverse | CAC GGC GGA GAT GTT GTT GAA GCG GTC GAT |
| | | | GGC ATT CGC AGG CGC CAT G (SEQ ID NO: 56) |
| 8 | seq3-F | Forward | ATCGACCGCTTCAACAACATCTCCGCCGTG (SEQ ID |
| | | | NO: 57) |
| 9 | pP18290-R4 | Reverse | CTT GAT CGA CTT CTC GAC CAG GTT CTC CAT GGC |
| | | | ATT CGC AGG CGC CAT G (SEQ ID NO: 58) |
| 10 | seq4-F | Forward | ATGGAGAACCTGGTCGAGAAGTCGATCAAG (SEQ ID |
| | | | NO: 59) |
| 11 | p18290-R5 | Reverse | CAG GCG CTC GAT CTT GAT GTT GGA GAT GTT GGC |
| | | | ATT CGC AGG CGC CAT G (SEQ ID NO: 60) |
| 12 | seq5-F | Forward | AACATCTCCAACATCAAGATCGAGCGCCTG (SEQ ID |
| | | | NO: 61) |
| 13 | p18290-R7 | Reverse | GCC GTT GCC CAT GGG GAT GTA GCG CAC CAT GGC |
| | | | ATT CGC AGG CGC CAT G (SEQ ID NO: 62) |
| 14 | seq7-F | Forward | ATGGTGCGCTACATCCCCATGGGCAACGGC (SEQ ID |
| | | | NO: 63) |
| 15 | p04850-F | Forward | GATCGGCACGTAAGAGGTTCCAACTTTCACCCCACC |
| | | | GTGAGCTGCACCT (SEQ ID NO: 64) |
| 16 | p04850-R1 | Reverse | GGC GCG CTT GTT CAG GGG GCC CTC CTG CAG GGC |
| | | | CTG TGC AAG TGG TGC AAA C (SEQ ID NO: 65) |
| 17 | p04850-R2 | Reverse | CCA CGA GTC CCA GGT GGC ACG CTT CGA GAT |
| | | | GGC CTG TGC AAG TGG TGC AAA C (SEQ ID NO: 66) |
| 18 | p04850-R3 | Reverse | CAC GGC GGA GAT GTT GTT GAA GCG GTC GAT |
| | | | GGC CTG TGC AAG TGG TGC AAA C (SEQ ID NO: 67) |
| 19 | p04850-R4 | Reverse | CTT GAT CGA CTT CTC GAC CAG GTT CTC CAT GGC |
| | | | CTG TGC AAG TGG TGC AAA C (SEQ ID NO: 68) |
| 20 | p04850-R5 | Reverse | CAG GCG CTC GAT CTT GAT GTT GGA GAT GTT GGC |
| | | | CTG TGC AAG TGG TGC AAA C (SEQ ID NO: 69) |
| 21 | p04850-R7 | Reverse | GCC GTT GCC CAT GGG GAT GTA GCG CAC CAT GGC |
| | | | CTG TGC AAG TGG TGC AAA C (SEQ ID NO: 70) |
| 22 | P08450-F | Forward | GATCGGCACGTAAGAGGTTCCAACTTTCACCTCGGTC |
| | | | GGGTCCGGACGGG (SEQ ID NO: 71) |
| 23 | P08450-R | Reverse | CAC GCG CGC CGT CTT GAC GAA GGG ATT CAC |
| | | | GTA GTA GGG CTC CGA TGT TGG ATG (SEQ ID NO: 72) |
| 24 | SP18250-F | Forward | CGGCCGCGGGAAGGACCAGACATGCTCACTCGCAAG |
| | | | AGAGTGG (SEQ ID NO: 73) |
| 25 | SP18250-R | Reverse | CCAGGTGGCACGCTTCGAGATGGGGCCGGTGGCGGC |
| | | | GCTG (SEQ ID NO: 74) |
| 26 | SeqX-SP | Reverse | GTT CTG GGC GGC CAG CTC GCG CAG GGT CGA CTC |
| | | | GGC CTG TGC AAG TGG TGC AAA C (SEQ ID NO: 75) |
| 27 | SeqX-F | Forward | GAGTCGACCCTGCGCGAGCTGGCCGCCCAGAAC |
| | | | (SEQ ID NO: 76) |
| 28 | seqX-R | Reverse | TCT ATT TCG TTC ATC CAT AGT TGC CTG ACT CGA |
| | | | GCA GGT GGC GCC GTT CAG GGT G (SEQ ID NO: 77) |
| 29 | SeqY-SP | Reverse | CAT GTT GGG GTT CAC GTA GAA GGG CGA GTC GTT |
| | | | GGC CTG TGC AAG TGG TGC AAA C (SEQ ID NO: 78) |
| 30 | SeqY-F | Forward | AACGACTCGCCCTTCTACGTGAACCCCAACATG (SEQ |
| | | | ID NO: 79) |
| 31 | SeqY-R | Reverse | TCT ATT TCG TTC ATC CAT AGT TGC CTG ACT CGA |
| | | | GGC GGC GCA GGT CAG GGT GGG C (SEQ ID NO: 80) |
| 32 | seqZ-SP | Reverse | CTG CAG GGC CTC GCC GTA GTT GTA GGT GCC GGC |
| | | | GGC CTG TGC AAG TGG TGC AAA C (SEQ ID NO: 81) |
| 33 | seqZ-F | Forward | GCCGGCACCTACAACTACGGCGAGGCCCTGCAG |
| | | | (SEQ ID NO: 82) |
| 34 | SeqZ-R | Reverse | TCT ATT TCG TTC ATC CAT AGT TGC CTG ACT GCC |
| | | | CTG GGG CAG CTG GGT GAT (SEQ ID NO: 83) |

### Example 4—Cloning Expression Cassettes (Inserts) into pOWR3 Vector, Expression in P. freudenreichii, and Examining Polysaccharide Degrading Activity in Various Substrates

The various amplified nucleotides sequences (expression cassettes) of example 3 (examples 3.1-3.17), were cloned into the pOWR3 vector using restriction free cloning method (Peleg Y, Unger T. “Application of the Restriction-Free (RF) cloning for multicomponents assembly”. Methods Mol Biol. 2014; 1116:73-87). The resulting vector was inserted and amplified in DH5alfa bacteria and then transferred to dam\dcm-E. coli strain, propagated and purified, to produce non-methylated vector.

The purified vector was inserted into P. freudenreichii using the electroporation process as described in Example 1. The resulting recombinant P. freudenreichii is examined for its ability to express and secrete polysaccharide degrading enzymes, and the resulting activity in degrading a suitable saccharide (such as, starch, cellulose and xylan),

Examining starch degrading activity in various substrates—The resulting recombinant P. freudenreichii is examined for its ability to express and secrete glucoamylase, and the resulting glucoamylase activity of degrading starch.

The recombinant P. freudenreichii colony is transferred to agar plates containing YEL media (gr/l: 10 yeast extract, 10 peptone, 10 lactate) and 0.5% starch, for overnight culture. Degradation of starch is detected by pouring Lugol solution on the agar plate to visualize the starch.

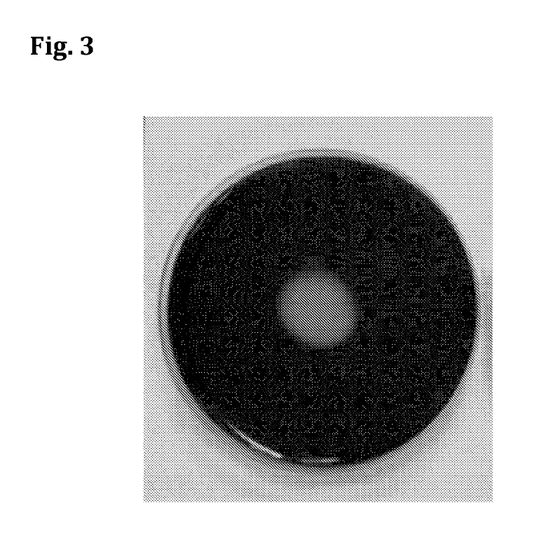

In the presence of starch, the Lugol forms black precipitate. In the presence of glucoamylase the starch is degraded and colorless halo is observed. A control Lugol assay is demonstrate in FIG. 3, which show an agar plate containing YEL media and 0.5% starch that is exposed to Lugol solution. All the areas containing starch are visualized in black, while a colorless circle, indicating that no starch is present can be seen in the area in which glucoamylase enzyme has diffused in the agar plate.

Additionally, the resulting recombinant (genetically engineered) P. freudenreichii is transferred to liquid YEL media (gr/l:10 yeast extract, 10 peptone, 10 lactate) containing 0.5-5% starch, for overnight culture in 30° C.−37° C., followed by culture in 55° C. for 2 hours. First, 0.5 mL of the post-incubations culture is tested by a colorimetric assay for its glucose content. In some cases, the assay is compared to a control culture of wild-type (non-modified) P. freudenreichii culture treated under the same conditions and/or to the same media, which does not include added bacteria. The secreted glucoamylase degrades the starch in the media and produces glucose that can be detected by a colorimetric method. Second, a similar assay is done, in which the post-incubations medium is being centrifuged and only the supernatant is examined for its glucose content. Further, the activity of the glucoamylase activity under optimal conditions is tested: 0.1 mL of the overnight culture is incubated with 0.4 mL of 1% starch solution for 2 hours, after which the tube is centrifuged and the supernatant is tested for its glucose content by a colorimetric method.

Additionally, 10̂8 CFU/ml of the recombinant P. freudenreichii is inoculated into a shredded organic waste (pH 5.5) for overnight culture in 30° C.-37° C., followed by culture in 55° C. for about 2 hours. 0.5 mL of the overnight culture is centrifuged and the supernatant is tested for its glucose content by a colorimetric method.

### Example 4.1—Expression of Glucoamylase Gi34146785 from Clostridium thermoamylolyticum Combined with a PFREUD_04850 Promoter and a PFREUD_04850 Signal Peptide in P. freudenreichii, and Examination of its Starch Degrading Activity

The expression cassette described in example 3.11 (SEQ ID NO: 43), was cloned into the pOWR3 vector and transformed into P. freudenreichii as described above.

The resulting recombinant P. freudenreichii was examined for its ability to express and secrete the Glucoamylase, and to test the glucoamylase activity of degrading starch. 10̂6-10̂9 CFU/ml of the resulting recombinant (genetically engineered) P. freudenreichii, or of corresponding wild-type bacteria (P. freudenreichii control), were transferred to liquid YEL media (gr/1:10 yeast extract, 10 peptone, 10 lactate) containing starch, for culture in 30-37° C., followed by additional culture in 55° C.

After the incubation periods, the cultures were verified for similar CFU content in the recombinant and control P. freudenreichii (for each temperature and/or CFU/ml bacteria). Each culture was tested by a colorimetric assay for its glucose content. A significant increase (over 35 fold) in glucose content was observed in the recombinant P. freudenreichii treated media, compared to the wild-type P. freudenreichii control.

These results indicate that the genetically engineered bacteria can successfully express and secrete an active polysaccharide degrading enzyme (Glucoamylase in this example) and that the secreted enzyme is active, as it is able to successfully degrade starch in the substrate to produce glucose.

### Example 5—Sequential Lactic Acid Utilization and Saccharification of Organic Waste

The LA-utilizing bacteria are used to utilize lactic acid and saccharify polysaccharide in organic waste, in a sequential mode of operation.

Grinding of mixed food waste is performed in an Optimum Commercial Blender in multiple batches that include 1000 gr food waste in 1000 ml of water. A semi-solid mixture of 10 L (working volume) is introduced into the fermenter (New Brunswick 15 L capacity). pH was measured at 5.2.

In-situ sterilization of the media (by autoclaving) is performed for 30 minutes at 121° C., with media agitation at 200 rpm. The fermenter is then cooled to 37° C. and temperature is maintained. Airflow is kept in head space to maintain positive pressure in the fermenter.

Lactic acid utilizing bacteria Starter preparation—inoculum for fermentation and lactic acid utilization by lactic acid utilizing bacteria (in this example, Propionibacterium freudenreichii (ATCC 9614)) is performed in a 1 L Shake flask, in Yeast-Extract Lactate (YEL) medium at low 100 rpm at 37° C. The cells are grown up to cell density of 1*10⁸.

Fermentation—15 ml is inoculated for 10.8 L fermentation, at 37° C., pH at 5.5 (controlled by 10% ammonia solution), until substantially complete utilization of lactic acid present in the media. Measurements of lactic acid and total glucose and fructose is performed. Broth sample are centrifuged at 6000 g for 10 min in 15° C. and supernatant is analyzed in a Reflectoquant analysis system (RQflex plus 10).

The fermenter is then heated to 55° C. for two hours to allow optimal conditions for activity of the polysaccharide degrading enzyme secreted by the bacteria (for example, Glucoamylase) and to allow sterilization of the bacteria (Propionibacterium freudenreichii). Following saccharification, the fermenter is cooled to 37° C., to allow further processing and production of discrete enantiomers of Lactic acid, by utilizing lactic acid producing bacteria (LAB).

LAB starter preparation—inoculum for L-lactate fermentation by Lactobacillus rhamnosus (ATCC 11443): 1 Liter Shake flask, conditions include MRS medium at 100 rpm agitation at 37° C. Cells are grown to a density of 1*10⁹. 15 ml of starter culture in inoculated for 10 L fermentation. Control temperature at 37° C., and pH at 6.5 by ammonia solution (10%). L-lactate fermentation is terminated as glucose is depleted from the media, typically after 24 to 48 hours.

### Example 6—Concomitant Utilization of Lactic Acid and Saccharification of Organic Waste

The LA-utilizing bacteria are used to concomitantly utilize lactic acid and saccharify polysaccharides in organic waste.

Grinding of mixed food waste is performed in an Optimum Commercial Blender in multiple batches that include 1000 gr food waste in 1000 ml of water. A semi-solid mixture of 10 L (working volume) is introduced into the fermenter (New Brunswick 15 L capacity). pH is measured at 5.2.

In-situ sterilization of the media (by autoclaving) is performed for 30 minutes at 121° C., with media agitation at 300 rpm. The fermenter is then cooled to 37° C. and temperature is maintained.

Lactic Acid utilizing bacteria Starter preparation—inoculum for fermentation and lactic acid utilization by lactic acid utilizing bacteria (in this example, Propionibacterium freudenreichii (ATCC 9614)) is performed in a 1 L Shake flask, in Yeast-Extract Lactate (YEL) medium at low 100 rpm at 37° C. The cells are grown up to cell density of 1*10⁸.

Fermentation—15 ml is inoculated for 10.8 L fermentation, at 37° C., pH at 5.5 (controlled by 10% ammonia solution), until substantially complete utilization of lactic acid presented in the media concomitant with saccharification of polysaccharide by the polysaccharide degrading enzyme secreted by the bacteria (for example, Glucoamylase). Measurements of lactic acid and total glucose and fructose is performed. Broth sample are centrifuged at 6000 g for 10 min in 15° C. and supernatant is analyzed in a Reflectoquant analysis system (RQflex plus 10).

The fermenter is heated to 55° C. for two hours to allow sterilization of the bacteria (Propionibacterium freudenreichii).

Thereafter, the fermenter is cooled to 37° C., to allow further processing and production of discrete enantiomers of Lactic acid, by utilizing lactic acid producing bacteria (LAB).

LAB starter preparation—inoculum for L-lactate fermentation by Lactobacillus rhamnosus (ATCC 11443): 1 Liter Shake flask conditions include MRS medium at 100 rpm agitation at 37° C. Cells are grown to density of 1*10⁹. 15 ml of starter culture is inoculated for 10 L fermentation. Control temperature at 37° C., and pH at 6.5 by ammonia solution (10%). L-lactate fermentation is terminated as glucose is depleted from the media, typically after 24 to 48 hours.

1.-45. (canceled)

46. A lactic acid (LA)-utilizing bacterium genetically modified to produce one or more exogenous polysaccharide-degrading enzyme, wherein the one or more exogenous polysaccharide-degrading enzyme comprises a cellulase, a hemicellulase, an amylase or combinations thereof.

47. The LA-utilizing bacterium of claim 46, wherein the polysaccharide-degrading enzyme is from a thermophilic bacterium selected from the group consisting of Clostridium sp., Paenibacillus sp., Thermobifida fusca, Bacillus sp., Geobacillus sp., Chromohalobacter sp. and Rhodothermus marinus.

48. The LA-utilizing bacterium of claim 46, wherein the polysaccharide-degrading enzyme is from a mesophilic bacterium is selected from the group consisting of Klebsiella sp., Cohnella sp., Streptomyces sp, Acetivibrio cellulolyticus, Ruminococcus albus; Bacillus sp. and Lactobacillus fermentum.

49. The LA-utilizing bacterium of claim 46, wherein the polysaccharide-degrading enzyme is from a fungus selected from the group consisting of Trichoderma reesei, Humicola insolens, Fusarium oxysporum, Aspergillus oryzae, Penicillium fellutanum and Thermomyces lanuginosu.

50. The LA-utilizing bacterium of claim 46, wherein the LA-utilizing bacterium is selected from the group consisting of Propionibacterium species, Megasphaera species, Selenomonas species and Veillonella species.

51. The LA-utilizing bacterium of claim 46, wherein the LA-utilizing bacterium is Propionibacterium freudenreichii.

52. A population of lactic-acid (LA)-utilizing bacteria genetically modified to produce a plurality of exogenous polysaccharide-degrading enzymes comprising a cellulase, a hemicellulase, an amylase or combinations thereof, wherein the population comprises a plurality of sub-populations of LA-utilizing bacteria, each sub-population being genetically modified to produce a different polysaccharide-degrading enzyme.

53. The population of LA-utilizing bacteria of claim 52, wherein the LA-utilizing bacteria comprise Propionibacterium freudenreichii.

54. A method for processing organic waste, the method comprising contacting the organic waste with the LA-utilizing bacterium of claim 46 under conditions in which lactic acid is consumed by the LA-utilizing bacteria and which are suitable for expression and activity of the polysaccharide-degrading enzymes, wherein said processing eliminates D-lactic acid, L-lactic acid or both from the organic waste and degrades polysaccharides in the waste to release soluble reducing sugars.

55. The method of claim 54, wherein elimination of lactic acid from the waste is effected concomitant with degradation of polysaccharides into soluble reducing sugars.

56. The method of claim 54, wherein elimination of lactic acid from the waste is effected prior to degradation of polysaccharides into soluble reducing sugars.

57. The method of claim 56, wherein the polysaccharide-degrading enzymes have suitable activity at a temperature higher than the suitable growth temperature of the LA-utilizing bacteria, and wherein the method comprises: (i) contacting the organic waste with the LA-utilizing bacteria at a first temperature, the first temperature being suitable for growth of the LA-utilizing bacteria and expression of the polysaccharide-degrading enzymes, for sufficient time to eliminate D-lactic acid, L-lactic acid or both from the waste; and (ii) increasing the temperature to a second temperature, the second temperature being suitable for activity of the polysaccharide-degrading enzymes.

58. The method of claim 56, wherein the polysaccharide-degrading enzymes have suitable activity at a pH higher or lower than the suitable growth pH of the LA-utilizing bacteria, and wherein the method comprises: (i) contacting the organic waste
